(19) United States
(12) Patent Application Publication
(10) Pub. No.: US 2022/0249627 A1
(43) Pub. Date: Aug. 11, 2022

(54) COMPOSITIONS AND METHODS FOR INHIBITING BIOLFILM DEPOSITION AND PRODUCTION

(71) Applicant: THE TRUSTEES OF THE UNIVERSITY OF PENNSYLVANIA, Philadelphia, PA (US)

(72) Inventors: Hyun Koo, Philadelphia, PA (US); Henry Daniell, Media, PA (US)

(73) Assignee: THE TRUSTEES OF THE UNIVERSITY OF PENNSYLVANIA, Philadelphia, PA (US)

(21) Appl. No.: 17/672,438

(22) Filed: Feb. 15, 2022

Related U.S. Application Data
(63) Continuation-in-part of application No. 16/301,023, filed on Nov. 13, 2018, now Pat. No. 11,246,819.
(60) Provisional application No. 62/335,650, filed on May 12, 2016.

(51) Int. Cl. A61K 38/47; A61K 47/36; A61K 47/69; A61P 31/00

(52) U.S. Cl. CPC A61K 38/47; A61P 31/00; A61K 47/6943; A61K 47/36

(57) ABSTRACT

The invention provides a method for combating biofilm, said method comprising contacting a surface at-risk for biofilm formation or a biofilm with a composition comprising an effective amount of antimicrobial peptide biofilm-degrading enzyme combinations, preferably in the form of a fusion protein. The biofilm may be on an animate or inanimate surface and both medical and non-medical uses and methods are provided. In one aspect the invention provides a composition for use in the treatment or prevention of a biofilm infection in a subject, particularly in the oral cavity.

### CROSS-REFERENCE TO RELATED APPLICATIONS

This application is a Continuation-in-part of U.S. application Ser. No. 16/301,023, filed Nov. 13, 2018, which is a § 371 of International Application No. PCT/US17/32437, filed May 12, 2017, which claims priority to U.S. Provisional Application No. 62/335,650 filed May 12, 2016, the entire disclosure of each of the foregoing applications being incorporated herein by reference as though set forth in full.

### GOVERNMENT SUPPORT STATEMENT

This invention was made with government support under Grant Nos: R01 HL107904 and R01 HL109442 awarded by the National Institutes of Health. The government has certain rights in the invention.

### INCORPORATION-BY-REFERENCE OF MATERIAL SUBMITTED IN ELECTRONIC FORM

Incorporated herein by reference in its entirety is the sequence listing submitted via EFS-Web as a text file named SEQLIST.txt, created Feb. 15, 2022, and having a size of 135,051 bytes.

### FIELD OF THE INVENTION

The present invention relates to the fields of biofilm deposition and the treatment of disease. More specifically, the invention provides compositions and methods useful for the treatment of dental caries and other oral diseases. The invention also provides methods for coating biomedical devices for inhibiting undesirable biofilm deposition thereon.

### BACKGROUND OF THE INVENTION

Several publications and patent documents are cited throughout the specification in order to describe the state of the art to which this invention pertains. Each of these citations is incorporated by reference herein as though set forth in full.

Biopharmaceuticals produced in current systems are prohibitively expensive and are not affordable for large majority of the global population. The cost of protein drugs ($140 billion in 2013) exceeds GDP of >75% of countries around the globe [Walsh 2014], making them unaffordable. One third of the global population earns <$2 per day and can't afford any protein drug (including the underprivileged, elderly and lower socio-economic groups in the US). Such high costs are associated with protein production in prohibitively expensive fermenters, purification, cold transportation/storage, short shelf life and sterile delivery methods [Daniell et al 2015, 2016].

Biofilms are formed by a complex group of microbial cells that adhere to the exopolysaccharide matrix present on the surface of medical devices. Biofilm-associated infections associated with medical device implantation pose a serious problem and adversely affects the function of the device. Medical implants used in oral and orthopedic surgery are fabricated using alloys such as stainless steel and titanium. Surface treatment of medical implants by various physical and chemical techniques has been attempted in order to improve surface properties, facilitate biointegration and inhibit bacterial adhesion as bacterial adhesion is associated with surrounding tissue damage and often results in malfunction of the implant.

Many infectious diseases in humans are caused by biofilms, including those occurring in the mouth [Hall-Stoodley et al., 2004; Marsh, et al 2011]. For example, dental caries (or tooth decay) continues to be the single most prevalent biofilm-associated oral disease, afflicting mostly underprivileged children and adults in the US and worldwide, resulting in expenditures of >$81 billion annually [Beiker and Flemmig, 2011; Dye et al., 2015; Kassebaum et al, 2015]. Caries-causing (cariogenic) biofilms develop when bacteria accumulate on tooth-surfaces, forming organized clusters of bacterial cells that are firmly adherent and enmeshed in an extracellular matrix composed of polymeric substances such as exopolysaccharides (EPS) [Bowen and Koo, 2011].

Additionally, aerosolized microbes generated during dental procedures and mechanical plaque/biofilm removal have been recognized as potential source for the spread of several infectious diseases (Bennett et al., 2000). This has received greater attention during the current COVID-19 global pandemic (Xu et al., 2020). The saliva and dorsum of the tongue are major sources of SARS-CoV-2 and stability of the virus in the aerosol and its spread in the aerosolized form has been widely reported (Van Doremalen et al., 2020; World Health Organization [WHO], 2020; Xu et al., 2020). Therefore, WHO and dental associations including American Dental Association recommended suspension of aerosol-generating procedures in the clinic except for emergencies (Bennett et al., 2000; Van Doremalen et al., 2020; WHO, 2020; Xu et al., 2020).

Furthermore, COVID-19 patients have shown high accumulation of pathogenic oral bacteria, whereas poor oral hygiene which disproportionately afflicts impoverished populations, may be a risk factor for COVID-19 (Patel and Sampson, 2020). Accumulation of microbes on teeth leads to the formation of intractable dental biofilms (plaque) that cause oral diseases such as dental caries (tooth decay) requiring costly clinical interventions at the dental office. Hence, development of alternative methods for plaque control at home is of paramount importance and urgency.

Current topical antimicrobial modalities for controlling cariogenic biofilms are limited. Chlorhexidine (CHX) is considered the ‘gold standard’ for oral antimicrobial therapy, but has adverse side effects including tooth staining and calculus formation, and is not recommended for daily therapeutic use [Jones, 1997; Autio-Gold, 2008]. As an alternative, several antimicrobial peptides (AMPs) have emerged with potential antibiofilm effects against caries-causing oral pathogens such as Streptococcus mutans [da Silva et al., 2012; Guo et al., 2015].

Antimicrobial peptides (AMP) are an evolutionarily conserved component of the innate immune response and are naturally found in different organisms, including humans. When compared with conventional antibiotics, development of resistance is less likely with AMPs. They are potently active against bacteria, fungi and viruses and can be tailored to target specific pathogens by fusion with their surface antigens (Lee et al 2011; DeGray et al 2001; Gupta et al 2015). Linear AMPs have poor stability or antimicrobial activity when compared to AMPs with complex secondary structures. For example, retrocyclin or protegrin have high antimicrobial activity or stability when cyclized (Wang et al 2003) or when forming a hairpin structure (Chen et al 2000) via disulfide bond formation. RC 101 is highly stable at pH 3, 4, 7 and at temperatures ranging from 25° C. to 37° C. as well as in human vaginal fluid for 48 hours (Sassi et al 2011a), while its antimicrobial activity was maintained for up to six months (Sassi et al 2011b). Likewise, protegrin is highly stable in salt or human fluids (Lai et al 2002; Ma et al 2015) but lost potency when linearized. These intriguing characteristics of antimicrobial peptides with complex secondary structures may facilitate development of novel therapeutics. However, the high cost of producing sufficient amounts of antimicrobial peptides as well as other biofilm degrading enzymes is a major barrier for their clinical development and commercialization.

### SUMMARY OF THE INVENTION

In accordance with the present invention, a multi-component composition comprising at least one antimicrobial peptide (AMP) and at least one biofilm degrading enzyme which act synergistically to degrade biofilm structures and inhibit biofilm deposition is provided. In certain embodiments, the AMP is selected from protegrin 1, RC-101 and the AMPs listed in Table 1. The biofilm degrading enzyme, includes, for example, mutanase, dextranase, glucoamylase, deoxyribonuclease I, DNAase, dispersin B, glycoside hydrolases and the enzymes provided in Table 2. In certain embodiments, the coding sequences for these enzymes are codon optimized for expression in a plant chloroplast. In a particularly preferred embodiment, the at least one AMP and at least one biofilm degrading enzyme are produced recombinantly. In a particularly preferred embodiment, the AMP and biofilm degrading enzyme(s) are expressed as a fusion protein. When the composition is for the treatment of oral diseases, the composition may optionally further comprise an antibiotic, fluoride, CHX or all of the above. The composition may be contained within chewing gum, hard candy, or within an oral rinse. Preferred fusion proteins of the invention include, without limitation, PG-1-Mut, PG-1-Dex, PG-1-Mut-Dex, RC-101-Mut, RC-101-Dex, RC-101-Mut-Dex for use alone or in combination for the degradation of biofilms. Notably any of the AMPs listed in Table 1 can replace either PG-1 or RC-101 in the aforementioned fusion proteins to alter or improve the bacteriocidal action of the fusion protein.

To alter the degradation activity of the fusion proteins, the enzymes listed above and hereinbelow may replace Mut, Dex or both in the fusion proteins of the invention. In another embodiment, when two different EPS enzymes are employed in the compositions, such enzymes may be delivered at different ratios, e.g., 1:2, 1:3, 1:4, 1:5, 1:6, 1:7, 1:8 etc. When Mut and Dex are delivered together in a gum or oral rinse for example, a preferred ratio is 5:1 Dex:Mut.

In another aspect, the invention comprises a biofilm degrading composition harboring a mutanase enzyme. in a pharmaceutically acceptable carrier. In preferred embodiments, the mutanase is encoded by SEQ ID NO: 1 and is expressed in a plant plastid. In certain embodiments, the composition further comprises a plant remnant. The plant remnant may be freeze dried. In a particularly preferred embodiment, the plant remnant is from tobacco or lettuce. In certain embodiments, the composition comprises at least one AMP, lipase, and/or biofilm degrading enzyme. In certain embodiments, the lipase is obtained from a commercial vendor or produced in a plant plastid present in said plant remnant. The biofilm degrading enzyme, includes, for example, dextranase, glucoamylase, deoxyribonuclease I, DNAase, dispersin B, glycoside hydrolases and the enzymes provided in Table 2. The at least one biofilm degrading enzyme may have a sequence selected from SEQ ID NOS: 2, 12, 14, 16, 18, 20, 24, and 26. In particularly preferred embodiments, the composition comprises mutanase, dextranase and lipase present in a chewing gum carrier. In certain embodiments, the dextranase and mutanase are present in a 5:1 ratio in said chewing gum.

In certain embodiments, the biofilm degrading composition further comprises an antimicrobial, an antibiotic, fluoride, and/or CHX. When the composition is for the treatment of oral diseases, the composition may be contained within chewing gum, hard candy, or within an an oral rinse.

In another aspect, the invention provides a method of degrading and/or removing biofilm comprising contacting a surface harboring said biofilm with the compositions described above, the composition having a bactericidal effect, and reducing or eliminating said biofilm comprising one or more undesirable microorganisms, wherein when said biofilm is present in or on an animal subject in need of said reduction or elimination. In certain embodiments, the biofilm is present in the mouth. In other embodiments, the biofilm is present on an implanted medical device. The method may also be used to remove biofilms present in an internal or external body surface is selected from the group consisting of a surface in a urinary tract, a middle ear, a prostate, vascular intima, heart valves, skin, scalp, nails, teeth and an interior of a wound. In particularly preferred embodiments, the composition used in these methods comprises mutanase, dextranase, and lipase in a suitable carrier.

In yet another embodiment, the composition of the invention comprises said at least one AMP and said at least one biofilm degrading enzyme are produced in a plant plastid. The plant may be a tobacco plant and the sequences encoding said AMP and enzyme is codon optimized for expression in a plant plastid. In a preferred embodiment, the AMP and biofilm degrading enzyme are expressed in a lettuce plant as a fusion protein under the control of endogenous regulatory elements present in lettuce plastids. In other embodiments, the composition does not comprise an AMP, but does contain lipase.

### BRIEF DESCRIPTION OF THE DRAWINGS

FIGS. 1A-1D—Purification of GFP fused Retrocyclin (RC101) and Protegrin (PG1) expressed in tobacco chloroplasts—FIG. 1A. Western blot analysis of purified GFP-RC101 fusion using Anti-GFP antibody. FIG. 1B. Native fluorescence gel of purified GFP-RC101 fusion. FIG. 1C. Western blot of purified GFP-PG1 fusion using Anti-GFP antibody. FIG. 1D. Native fluorescence gel of purified GFP-PG1. Note—All the samples for FIG. 1A-1D were loaded based on total protein values obtained from the Bradford method. Densitometry using Image J software was done to determine GFP concentration Expression level, purity and yield. Expression level and yield were calculated from GFP concentrations relative to total protein values. Yield was determined by multiplying GFP concentration with recovered volume after purification. Individual peptide yield was determined by dividing GFP yield with molar factor 14 (ratio of GFP MW to peptide MW). The fold enrichment was calculated by dividing % purity with % expression in plant crude extracts.

FIGS. 2A-2E. Antimicrobial activity of AMPs (GFP-PG1 and GFP-RC101) against Streptococcus mutans and other oral microbes. Cell viability was determined by absorbance (A₆₀₀ₙₘ) and counting colony forming units (CFU) over-time. (FIG. 2A) Time-killing curve of S. mutans treated with different concentrations of GFP-PG1 and synthetic PG1 (A600 nm). (FIG. 2B) Viable cells (CFU/ml) of S. mutans treated with GFP-PG1 and synthetic PG1 at each time point. (FIG. 2C) Time-killing curve of S. mutans treated with GFP-RC101 at different concentrations (A₆₀₀ₙₘ). (FIG. 2D) Viable cells (CFU/ml) of S. mutans treated with GFP-RC101 at each time point. (FIG. 2E) Viable cells (CFU/ml) of S. gordonii, A. naeslundii and C. albicans treated with GFP-PG1 at 10 μg/ml for 1 h and 2 h.

FIGS. 3A-3C. Bacterial killing by GFP-PG1 as determined via confocal fluorescence and SEM imaging (FIG. 3A) Time-lapse killing of S. mutans treated with GFP-PG1 at 10 μg/ml. The control group (FIG. 3B) consisted of S. mutans cells treated with buffer only. Propidium iodide (PI) (in red) was used with confocal microscopy to determine the bacterial viability over time at single-cell level. PI is cell-impermeant and only enters cells with damaged membranes; in dying and dead cells a bright red fluorescence is generated upon binding of PI to DNA. GFP-PG1 is shown in green. (FIG. 3C) Morphological observations of S. mutans subjected to GFP-PG1 at a concentration of 10 μg/ml for 1 h using scanning electron microscopy. Red arrows show dimpled membrane and extrusion of intracellular content.

FIGS. 4A-4C Inhibition of biofilm formation by a single topical treatment of GFP-PG1. This figure displays representative images of three-dimensional (3D) rendering of S. mutans biofilm. Bacterial cells were stained with SYTO 9 (in green) and EPS were labeled with Alexa Fluor 647 (in red). Saliva-coated hydroxyapatite (sHA) disc surface was treated with a single topical treatment of GFP-PG1 with a short-term 30 min exposure (FIG. 4B). The control group (FIG. 4A) was treated with buffer only. Then, the treated sHA disc was transferred to culture medium containing 1% (w/v) sucrose and actively growing S. mutans cells (10⁵ cfu/ml) and incubated at 37° C., 5% CO₂ for 19 h. After biofilm growth, the biofilms were analyzed by two photon confocal microscopy. (FIG. 4C) Quantitative analysis of proportion of live and dead S. mutans cells via quantitative PCR (qPCR) with or without propidium monoazide (PMA) treatment (Klein et al., 2012). The combination of PMA and qPCR (PMA-qPCR) quantify viable cells with intact membrane. Before genomic DNA isolation and qPCR quantification, PMA is added to selectively cross-link DNA of dead cells, and thereby prevent PCR amplification (Klein et al., 2012). Asterisks indicate that the values from GFP-PG1 treatment are significantly different from control (P<0.05).

FIG. 5. EPS-degrading enzymes digesting biofilm matrix. Representative time-lapsed images of EPS degradation in S. mutans biofilm treated with combination of dextranase and mutanase. Bacterial cells were stained with SYTO 9 (in green) and EPS were labeled with Alexa Fluor 647 (in red). The white arrows show ‘opening’ of spaces between the bacterial cell clusters and ‘uncovering’ cells following enzymatic degradation of EPS.

FIGS. 6A-6C. Biofilm disruption by synthetic PG1 alone or in combination with EPS-degrading enzymes. (FIG. 6A) Time-lapse quantification of EPS degradation within intact biofilms using COMSTAT. (FIG. 6B) The viability of S. mutans biofilm treated with synthetic PG1 and EPS-degrading enzymes (Dex/Mut) either alone or in combination by ImageJ. (FIG. 6C) Antibiofilm activity of synthetic PG1 was enhanced by EPS-degrading enzymes (Dex/Mut). Asterisks indicate that the values for different experimental groups are significantly different from each other (P<0.05).

FIG. 7. In vitro uptake of purified fused protein CTB-GFP, PTD-GFP, Protegrin-1-GFP (PG1-GFP) and Retrocyclin101-GFP (RC101-GFP) in different human periodontal cell lines: human periodontal ligament stem cells (HPDLS), maxilla mesenchymal stem cells (MMS), human head and neck squamous cell carcinoma cells (SCC), gingiva-derived mesenchymal stromal cells (GMSC), adult gingival keratinocytes (AGK) and osteoblast cell (OBC) with confocal microscopy. 2×10⁴ cells of human cell lines HPDLS, MMS, SCC, GMSC, AGK and OBC were cultured in 8-well chamber slides (Nunc) at 37° C. for overnight, followed by incubation with purified GFP fusion proteins: CTB-GFP (8.8 μg), PTD-GFP (13 μg), PG1-GFP (1.2 μg), RC101-GFP (17.3n) in 100 μl PBS supplemented with 1% FBS, respectively, at 37° C. for 1 hour. After fixing with 2% paraformaldehyde at RT for 10 min and washing with PBS for three times, the cells were stained with antifade mounting medium with DAPI. For negative control, cells were incubated with commercial GFP (2 μg) in PBS with 1% FBS and processed in the same condition. All fixed cells were imaged using confocal microscope. The green fluorescence shows GFP expression; the blue fluorescence shows DAPI labeled cell nuclei. The images were observed under 100× objective, and at least 10-15 GFP-positive cells or images were observed in each cell line. Scale bar represent 10 μm. All images studies have been analyzed in triplicate.

FIG. 8. Downstream processing of GFP fusions from transplastomic tobacco: Flow diagram illustrating the different steps involved in generation of purified GFP fusions from transplastomic tobacco plants grown in greenhouse.

FIGS. 9A-9B. Vectors and codon optimized sequences for mutanase (FIG. 9A) and dextranase (FIG. 9B). Codon optimized mutanase: SEQ ID NO: 1. Codon Optimized dextranase: SEQ ID NO: 2.

FIG. 10. A schematic diagram of a chloroplast vector expressing tandem repeats of AMPs fused with GVGVP (SEQ ID NO: 11) for use alone or for expressing fusion proteins comprising the EPS proteins in FIG. 9.

FIG. 11. Novel purification strategy: inverse temperature cycling purification process demonstrates high yield.

FIGS. 12A-12B: Expression of functional codon optimized mutanase in E. coli. FIG. 12 shows western blots showing mutanase expression in E. coli. FIG. 12B shows E. coli spread on 0.5% blue dextran plates. Transformed clones are able to produce recombinant dextranase normally made in S. mutans and able to clear a blue halo around the colony. FIG. 12C represents a gel diffusion assay comparing the degradation activity of recombinant dextranase present in the crude lysate (Total Protein loading) from the transformed E. coli against blue dextran as compared to commercially purified enzyme from Penicillin.

FIG. 13. A flow diagram of the steps for engineering lettuce plants for AMP/biofilm degrading enzyme production.

FIG. 14. Chewing gum tablet preparation is shown. While GFP is exemplified herein, chewing gum comprising the AMP-enzyme fusion proteins (e.g., those provided in FIGS. 9 and 10) is also within the scope of the invention.

FIG. 15. Gum tablets were evaluated via fluorescence, and by western blot to ascertain the concentration of GFP. Quantification of the GFP release from chewing gum based on (i) Western blotting (ii) Fluorometer (Fluoroskan Ascent™ Microplate Fluorometer—Thermo; λₑₓ 485 nm; λₑₘ, 538 nm). Commercial GFP (Vector Laboratories, Cat #MB-0752) was used as standard. The chewing gum was ground in the protein extraction buffer.

FIG. 16. A chewing simulator is shown which uses artificial saliva for assessing release kinetics of biofilm degrading agents from the gum tablets of the invention.

FIG. 17. A graph showing quantification of GFP released from chewing gum. Gum tablets comprising increasing concentrations of GFP expressed in lettuce leaves were assessed in a chewing simulator in the presence of artificial saliva to determine GFP release kinetics.

FIG. 18. A graph demonstrating that crude extracts comprising enzymes expressed from chloroplast vectors are as efficacious for inhibiting CFU formation as commercial enzymes, when mixed with Listerine®. Enzymatic degradation of in vitro S. mutans biofilms using E. coli derived Mutanase and Dextranase (ratio 1:5) supplemented with Listerine®. Commercial Mutanase (from Bacillus sp., Amano) and Dextranase (from Penicillium sp., Sigma) was used as positive control while the crude E. coli extract served as negative control. CFU/ml is expressed as mean±standard deviation (n=2). ***, P<0.001 versus E. coli extract.

FIG. 19A-19B. In vitro cariogenic plaque biofilm model and the topical treatment regimen used. (FIG. 19A) A saliva-coated hydroxyapatite (sHA; a tooth surrogate) biofilm model which mimics bacterial-fungal interactions under cariogenic conditions. (FIG. 19B) Regimen of the topical biofilm treatments using commercial/plant-derived enzymes. The sHA surface was treated with plant-derived enzyme crude extract prior to biofilm formation followed by a second treatment (after 6 h) using the same extract to simulate topical oral applications.

FIG. 20A-20D. Generation of Marker-free (MF) lettuce plants expressing dextranase, mutanase and lipase and evaluation of transgene integration, marker removal and homoplasmy. (FIG. 20A) Schematic representation of the integration of two expression cassettes (gene of interest—GOI and selectable marker) into lettuce chloroplast genome via homologous recombination of flanking sequences: 16S rRNA-trnI and tranA-235 rRNA and subsequent removal of the antibiotic resistance gene via homologous recombination between two identical atpB regions. GOI represents PG1-Smdex or mut or lipY. Probe indicates the DNA fragment region which was digested by Bam HI and used to detect hybridizing fragments in Southern blots. (FIG. 20B) Southern blots confirm PG1-Smdex gene integration, marker removal and homoplasmy in transplastomic plants with 10.5 kb with 2.2 kb fragments, while 12.5 kb with 10.5 kb and 2.2 kb demonstrated heteroplasmy (with or without the aadA gene) after gDNA was digested with HindIII. Untransformed plant (WT) and undigested DNA (UD) were used as controls. (FIG. 20C) In MF mutanase T0 generation, expected bands of 14.1 or 16.1 kb as a result of HindIII digested gDNA confirm mut gene integration in T0 generation plants, and the 14.1 kb band alone represents the homoplasmy. (FIG. 20D) Expected band size of 5.6 kb obtained from SmaI digested gDNA confirms lipY gene integration, antibiotic marker gene removal and homoplasmy in lipase expressing T1 generation plants. Gene of interest band size is represented with arrowheads.

FIG. 21A-21E. Chloroplast derived Marker-free PG1-dextranse and mutanase enzyme activity. Enzyme extracted from Marker-free PG1-dextranse lyophilized leaf powder from harvested leaves and evaluated for the qualitative assay against 0.5% blue dextran on plate (FIG. 21A), quantitative enzyme assay against 1% dextran (FIG. 21B), release of enzyme in the plant crude extract with or without sonication (FIG. 21C), and enzyme stability evaluation of protein extracted in presence/absence of protease inhibitors (FIG. 21D). Marker-free mutanase enzyme activity (FIG. 21E). Enzyme activity calculated by measuring released reducing sugars and compared with the maltose standard. Assays were performed in triplicates and data represents mean and standard deviation. Statistical significance analysed by t-test. Statistical significance was set at P<0.05 (*), and P<0.001 (***). WT represents untransformed wild-type plant (-ve control); NS represents not significant.

FIG. 22A-22B. Chloroplast derived lipase activity against p-Nitrophenyl butyrate. Enzyme extracted from lyophilized leaf powder with or without sonication (FIG. 22A) and in presence or absence of protease inhibitors (FIG. 22B). Enzyme assay performed by incubating crude extract with substrate at 37° C. for 10 min, and enzyme units calculated by measuring released pNP and compared with the pNP standards. Data represent average and standard deviation.

FIG. 23A-23J. Anti-biofilm effects of commercial and plant-derived enzymes against bacterial-fungal mixed biofilms. Commercial purified enzymes of the same activity unit as measured in the plant crude extracts (333.3 U/mL for lipase and 7.08/0.84 U/mL for dextranase/mutanase, respectively) were used as standards to evaluate the antibiofilm efficacy of the plant crude extracts. (FIG. 23A) Confocal images showing the antibiofilm efficacy of commercial and plant-derived lipase. Yellow arrows show complete inhibition of hyphae formation; white arrows shows bacterial dispersion (FIG. 23B-23E) Quantitative computational analysis of the confocal biofilm images treated with commercial and plant-derived lipase. (FIG. 23F) Confocal images showing the antibiofilm efficacy of commercial and plant-derived Dextranase/Mutanase combination. Yellow arrows show the presence of hyphae; white arrows show complete degradation of EPS (FIG. 23G-23J) Quantitative computational analysis of the confocal biofilm images treated with commercial and plant-derived Dextranase/Mutanase combination. For multi-channel confocal images, C. albicans cells (yeasts or hyphae) are depicted in cyan; S. mutans cells are depicted in green; The EPS glucan matrix is depicted in red. For the computational data, the title of each graph indicates the channel(s) used for individual analysis. *, P<0.05; **, P<0.01 (one-way analysis of variance with Tukey's multiple comparisons test). Enzyme units of lipase and dextranase/mutanase represent μmol of pNP and reducing sugar produced in 1 h, respectively.

FIG. 24. Inhibition of hyphal formation in the C. albicans monoculture as a result of lipase topical treatment.

FIG. 25A-25E. Inhibition of fungal-bacterial mixed biofilm formation by topical sequential treatment of commercial Lipase and Dextranse/Mutanse combination. Commercial enzymes of the optimum activity units for bioactivity (1000 U/mL for lipase and 525/105 U/mL for dextranase/mutanase, respectively) were used. (FIG. 25A) Three-dimensional confocal images of the fungal-bacterial mixed biofilm formed after the topical sequential treatments. C. albicans cells (yeasts or hyphae) are depicted in cyan; S. mutans cells are depicted in green; The EPS glucan matrix is depicted in red. Representative merged biofilm images are displayed in the middle panel, while a magnified (close-up) view of each small box is positioned in the left panel. Lateral (side) views of each biofilm are displayed at the right panel (the merged image at the top and the EPS channel at the bottom). (FIG. 25B-25E) Quantitative computational analysis of the confocal images. The title of each graph indicates the channel(s) used for individual analysis. *P<0.05; **P<0.01 (one-way analysis of variance with Tukey's multiple comparisons test). Enzyme unit of lipase and dextranase/mutanase represent μmol of pNP and reducing sugar produced in 1 h, respectively.

FIG. 26A-26B. Viability of the fungal-bacterial mixed biofilm after sequential treatments with commercial Lipase and Dextranse/Mutanse combination. (FIG. 26A) Total Biofilm Inhibition (TBI) index of the treatments. TBI=I fungal CFU×I bacterial CFU×IDW, where Inhibition of bacterial/fungal CFU or ICFU=(CFUtreatment/CFUcontrol)×100%, and Inhibition of Dry Weight or IDW=(DWtreatment/DWcontrol)×100%. *, P<0.05; **, P<0.01 (one-way analysis of variance with Tukey's multiple comparisons test). (FIG. 26B) Live/dead staining of the fungal/bacteria mixed biofilms. Live cells are depicted in green; Dead cells are depicted in magenta; white arrows show killing of fungal yeast cells. C. albicans cell wall is depicted in cyan to indicate the location of fungal cells. The optimum activity units (U) were used for commercial purified lipase (1000 U/mL) and Dex/Mut (525/105 U/mL) in the experiments. Enzyme unit of lipase and dextranase/mutanase represent μmol of pNP and reducing sugar produced in 1 h, respectively.

FIG. 27A-27D. Anti-plaque chewing gum comprising enzymes expressed in chloroplasts. (FIG. 27A) Current plaque control modalities and the chewing gum prototype using the chloroplast technology. Conventional mechanical brushing/flossing requires appropriate cumbersome techniques and suffers from low compliance. Chemical approaches e.g., antimicrobial mouthwash has limited efficacy against cariogenic dental plaque, and are costly. Dental clinic tooth cleaning generates significant amounts of droplets and aerosols, posing potential risks of infection transmission, including COVID-19. (FIG. 27B) Steps in creation and mass production of lettuce plants expressing enzymes. (FIG. 27C) Estimation of the GFP release from the 25 mg chewing gum tablet. The GFP released in saliva and the remaining pellet after grinding at 1, 5, 7, and 10 min time points. (FIG. 27D) GFP activity remained stable in chewing gum tablets containing 25, 50, 75 and 100 mg lyophilized plant powder after storage at room temperature for 3 years.

FIG. 28A-28C. Paenibacillus sp. native mut gene codon frequency vs. codon optimized gene frequency. The gene codon was optimized based on the codon frequency of plant chloroplast psbA gene.

### DETAILED DESCRIPTION OF THE INVENTION

Many infectious diseases in humans are caused by virulent biofilms, including those occurring within the mouth (e.g. dental caries and periodontal diseases). Dental caries (or tooth decay) continues to be the single most costly and prevalent biofilm-associated oral disease in the US and worldwide. It afflicts children and adults alike, and is a major reason for emergency room visits leading to absenteeism from work and school. Unfortunately, the prevalence of dental caries is still high (>90% of US adult population) and it remains the most common chronic disease afflicting children and adolescents, particularly from a poor socio-economic background. Furthermore, poor oral health often leads to systemic consequences and impacts overall health. Importantly, the cost to treat the ravages of this disease (e.g. carious lesions and pulpal infection) exceeds $40 billion/yr in the US alone. Fluoride is the mainstay of dental caries prevention. However, its widespread use offers incomplete protection against the disease.

Fluoride is effective in reducing demineralization and enhancing demineralization of early carious lesions, but has limited effects against biofilms. Conversely, current antimicrobial modalities for controlling caries-causing biofilms are largely ineffective.

There is an urgent need to develop efficacious therapies to control virulent oral biofilms. In accordance with the present invention, methods for low-cost production and delivery of therapeutically effective plant-expressed biopharmaceuticals superior to current antibiofilm/anti-caries modalities are provided.

### Definitions

As used herein, antimicrobial peptides are small peptides having any bacterial activity. “RC-101” is an analogue of retrocyclin, a cyclic octadecapeptide, which can protect human CD4+ cells from infection by T- and M-tropic strains of HIV-1 in vitro and prevent HIV-1 infection in human cervicovaginal tissue. The ability of RC-101 to prevent HIV-1 infection and retain full activity in the presence of vaginal fluid makes it a good candidate for other topical microbicide applications, especially in oral biofilms. The sequence of RC-101 is provided in Plant Biotechnol J. 2011 January; 9(1): 100-115 which is incorporated herein by reference.

“C16G2” is a novel synthetic antimicrobial peptide with specificity for S. mutans,

“Protegrin-1 (PG)” is a cysteine-rich, 18-residue β-sheet peptide. It has potent antimicrobial activity against a broad range of microorganisms, including bacteria, fungus, virus, and especially some clinically relevant, antibiotic-resistant bacteria. For example, bacterial pathogens E. coli and fungal opportunist C. albicans are effectively killed by PG in laboratory testing. The sequence of PG-1 is provided in Plant Biotechnol J. 2011 January; 9(1): 100-115 which is incorporated herein by reference.

Additional antimicrobial peptides include those set forth below in Table 1 below.

TABLE 1
Peptide sequences (single letter amino acid
code) of CSP, CSP_{C16}-containgin STAMPS, and
STAMP components

| | | Molecular |
|---|---|---|
| | | wt |
| Peptide | Amino acid sequencea | (observed) |
| CSP | SGSLSTFFREENRSFTQALGK | 2,364.9 |
| CSP_{C16} | TFERLFNRSETQALGK | 1,933.3 |
| | (SEQ ID NO: 3) | |
| G2 | KNLRIIRKGIHIIKKYb | 1,993.5 |
| | (SEQ ID NO: 4) | |
| C16G2 | THPRLFNRSIPTOALGISIGGGKNLRII | 4,079.0 |
| | RKGIHIIKKYb (SEQ ID NO: 5) | |
| CSP_{MS} | THRLENR (SEQ ID NO: 6) | 1,100.6 |
| M8G2 | THRLFNRGGGKNLRIIRKGIHIIKKYb | 3,246.9 |
| | (SEQ ID NO: 7) | |
| S6L3-33 | FIKKFWKWFRRF (SEQ ID NO: 8) | 1,677.5 |
| C16-33 | TRRIZLFNIZSETQALGKSGGGFKKFWK | 1849.0 |
| | WFRRF(SEQ ID NO: 9) | |
| M8-33 | TFFRIAPNRSGGGFKKFWKWFRRF | 3,016.9 |
| | (SEQ ID NO: 10) | |

[symbol]

a Linker regions between targeting and killing peptides are underlined, [symbol]
b Peptide C-terminal arriidation,

A “biofilm” is a complex structure adhering to surfaces that are regularly in contact with water, consisting of colonies of bacteria and usually other microorganisms such as yeasts, fungi, and protozoa that secrete a mucilaginous protective coating in which they are encased. Biofilms can form on solid or liquid surfaces as well as on soft tissue in living organisms, and are typically resistant to conventional methods of disinfection. Dental plaque, the slimy coating that fouls pipes and tanks, and algal mats on bodies of water are examples of biofilms. Biofilms are generally pathogenic in the body, causing such diseases as dental caries, cystic fibrosis and otitis media.

“Biofilm degrading enzymes” include, without limitation, exo-polysaccharide degrading enzymes such as dextranase, mutanase, DNAse, endonuclease, deoxyribonuclease I, dispersin B, and glycoside hydrolases, such as 1→3)—α-D-glucan hydrolase, although use of chloroplast codon optimized sequences encoding dextranase and mutanase are preferred, the skilled person is well aware of many different biofilm degrading enzymes in the art. Additional enzyme sequences for use in the fusion proteins of the invention are provided below.

As used herein, the terms “administering” or “administration” of an agent, drug, or peptide to a subject includes any route of introducing or delivering to a subject a compound to perform its intended function. The administering or administration can be carried out by any suitable route, including orally, topically, intranasally, parenterally (intravenously, intramuscularly, intraperitoneally, or subcutaneously), or rectally. Administering or administration includes self-administration and the administration by another.

As used herein, the terms “disease,” “disorder,” or “complication” refers to any deviation from a normal state in a subject.

As used herein, by the term “effective amount” “amount effective,” or the like, it is meant an amount effective at dosages and for periods of time necessary to achieve the desired result.

As used herein, the term “inhibiting” or “preventing” means causing the clinical symptoms of the disease state not to worsen or develop, e.g., inhibiting the onset of disease, in a subject that may be exposed to or predisposed to the disease state, but does not yet experience or display symptoms of the disease state.

As used herein, the term “expression” in the context of a gene or polynucleotide involves the transcription of the gene or polynucleotide into RNA. The term can also, but not necessarily, involves the subsequent translation of the RNA into polypeptide chains and their assembly into proteins.

A plant remnant may include one or more molecules (such as, but not limited to, proteins and fragments thereof, minerals, nucleotides and fragments thereof, plant structural components, etc.) derived from the plant in which the protein of interest was expressed. Accordingly, a composition pertaining to whole plant material (e.g., whole or portions of plant leafs, stems, fruit, etc.) or crude plant extract would certainly contain a high concentration of plant remnants, as well as a composition comprising purified protein of interest that has one or more detectable plant remnants. In a specific embodiment, the plant remnant is rubisco.

In another embodiment, the invention pertains to an administrable composition for treating or preventing biofilm formation in situ (e.g., in the mouth) and on biomedical devices useful for surgical implantation such as stents, artificial joints, and the like. In this embodiment, the devices are coated with the composition to inhibit unwanted biofilm deposition on the device. The composition comprises a therapeutically-effective amount of one or more antimicrobial peptides (AMP) and one or more enzymes having biofilm degrading activity in combination, each of said AMP and enzyme thereof having been expressed by a plant and a plant remnant and acting synergisticall to degrade said biofilm. In certain embodiments the AMP(s) and enzymes(s) are expressed from separate plastid transformation vectors. In other embodiments, the plastid transformation vectors comprising polycistronic coding sequences where both the AMP and the enzymes are expressed from a single vector.

Proteins expressed in accord with certain embodiments taught herein may be used in vivo by administration to a subject, human or animal in a variety of ways. The pharmaceutical compositions may be administered orally, topically, subcutaneously, intramuscularly or intravenously, though oral topical administration is preferred.

Oral compositions produced by embodiments of the present invention can be administered by the consumption of the foodstuff that has been manufactured with the transgenic plant producing the plastid derived therapeutic protein. The edible part of the plant, or portion thereof, is used as a dietary component. The therapeutic compositions can be formulated in a classical manner using solid or liquid vehicles, diluents and additives appropriate to the desired mode of administration. Orally, the composition can be administered in the form of tablets, capsules, granules, powders, gums, and the like with at least one vehicle, e.g., starch, calcium carbonate, sucrose, lactose, gelatin, etc. The therapeutic protein(s) of interest may optionally be purified from a plant homogenate. The preparation may also be emulsified. The active ingredient is often mixed with excipients which are pharmaceutically acceptable and compatible with the active ingredient. Suitable excipients are, e.g., water, saline, dextrose, glycerol, ethanol or the like and combination thereof. In addition, if desired, the compositions may contain minor amounts of auxiliary substances such as wetting or emulsifying agents, pH buffering agents, or adjuvants. In a preferred embodiment the edible plant, juice, grain, leaves, tubers, stems, seeds, roots or other plant parts of the pharmaceutical producing transgenic plant is ingested by a human or an animal thus providing a very inexpensive means of treatment of disease.

In a specific embodiment, plant material (e.g. lettuce material) comprising chloroplasts expressing AMPs and biofilm degrading enzymes and combinations thereof, is homogenized and encapsulated. In one specific embodiment, an extract of the lettuce material is encapsulated. In an alternative embodiment, the lettuce material is powderized before encapsulation. As mentioned previously, the biofilm degrading proteins may also be purified from the plant following expression.

In alternative embodiments, the compositions may be provided with the juice of the transgenic plants for the convenience of administration. For said purpose, the plants to be transformed are preferably selected from the edible plants consisting of tomato, carrot and apple, among others, which are consumed usually in the form of juice.

According to another embodiment, the subject invention pertains to a transformed chloroplast genome that has been transformed with a vector comprising a heterologous gene that expresses a combination of peptides as disclosed herein.

Of particular interest is a transformed chloroplast genome transformed with a vector comprising a heterologous gene that expresses one or more AMP and biofilm degrading enzyme or a combination thereof, polypeptide. In a related embodiment, the subject invention pertains to a plant comprising at least one cell transformed to express a peptide as disclosed herein.

Reference to genetic sequences herein refers to single- or double-stranded nucleic acid sequences and comprises a coding sequence or the complement of a coding sequence for polypeptide of interest. Degenerate nucleic acid sequences encoding polypeptides, as well as homologous nucleotide sequences which are at least about 50, 55, 60, 65, 60, preferably about 75, 90, 96, or 98% identical to the cDNA may be used in accordance with the teachings herein polynucleotides. Percent sequence identity between the sequences of two polynucleotides is determined using computer programs such as ALIGN which employ the FASTA algorithm, using an affine gap search with a gap open penalty of −12 and a gap extension penalty of −2. Complementary DNA (cDNA) molecules, species homologs, and variants of nucleic acid sequences which encode biologically active polypeptides also are useful polynucleotides.

Variants and homologs of the nucleic acid sequences described above also are useful nucleic acid sequences. Typically, homologous polynucleotide sequences can be identified by hybridization of candidate polynucleotides to known polynucleotides under stringent conditions, as is known in the art. For example, using the following wash conditions: 2×SSC (0.3 M NaCl, 0.03 M sodium citrate, pH 7.0), 0.1% SDS, room temperature twice, 30 minutes each; then 2×SSC, 0.1% SDS, 50° C. once, 30 minutes; then 2×SSC, room temperature twice, 10 minutes each, homologous sequences can be identified which contain at most about 25-30% basepair mismatches. More preferably, homologous nucleic acid strands contain 15-25% basepair mismatches, even more preferably 5-15% basepair mismatches.

Species homologs of polynucleotides referred to herein also can be identified by making suitable probes or primers and screening cDNA expression libraries. It is well known that the Tm of a double-stranded DNA decreases by 1-1.5° C. with every 1% decrease in homology (Bonner et al., J. Mol. Biol. 81, 123 (1973). Nucleotide sequences which hybridize to polynucleotides of interest, or their complements following stringent hybridization and/or wash conditions also are also useful polynucleotides. Stringent wash conditions are well known and understood in the art and are disclosed, for example, in Sambrook et al., MOLECULAR CLONING: A LABORATORY MANUAL, 2^{nd} ed., 1989, at pages 9.50-9.51.

The following materials and methods are provided to facilitate the practice of the present invention.

### Microorganisms and Growth Conditions

Streptococcus mutans UA159 serotype c (ATCC 700610), Actinomyces naeslundii ATCC 12104, Streptococcus gordonii DL-1 and Candida albicans SC5314 were used in present study. These strains were selected because S. mutans is a well-established, virulent cariogenic bacteria [Ajdić D et al, 2002]. S. gordonii is a pioneer colonizer of dental biofilm, and A. naeslundii is also detected during the early stages of dental biofilm formation and may be associated with development of root caries [Dige I et al, 2009]. C. albicans is a fungal organism that colonizes human mucosal surfaces, and it is also detected in dental plaque from toddlers with early childhood caries [Hajeshengallis E et al, 2015]. All strains were stored at −80° C. in tryptic soy broth containing 20% glycerol. Blood agar plates were used for cultivating S. mutans, S. gordonii and A. naeslundii. Sabouraud agar plates were used for C. albicans. All these strains were grown in ultra-filtered (10 kDa molecular-weight cut-off membrane; Prep/Scale, Millipore, MA) buffered tryptone-yeast extract broth (UFTYE; 2.5% tryptone and 1.5% yeast extract, pH 7.0) with 1% glucose to mid-exponential phase (37° C., 5% CO₂) prior to use.

### Creation of Transplastomic Lines Expressing Different Tagged GFP Fusion Proteins

The transplastomic plants expressing GFP fused with CTB, PTD, retrocyclin and protegrin were created as described in previous studies [Limaye et al 2006; Kwon et al 2013; Xiao et al 2016; Lee et al 2011]. Transplastomic lines expressing GFP fusion proteins were confirmed using Southern blot assay as described previously [Verma et al 2008]. Also, expression of GFP tagged proteins were confirmed by visualizing green fluorescence from the leaves of each construct under UV illumination.

### Purification of Tag-Fused GFP Proteins

Purification of GFP fusions Protegrin-1 (PG1) and Retrocyclin (RC101) from transplastomic tobacco was accomplished by organic extraction followed by hydrophobic chromatography done previously (Lee et al, 2011). About 0.2-1 gm of lyophilized leaf material was taken and reconstituted in 10-20 ml of plant extraction buffer (0.2M Tris HCl pH 8.0, 0.1M NaCl, 10 mM EDTA, 0.4M sucrose, 0.2% Triton X supplemented with 2% Phenylmethylsulfonylfluoride and 1 protease inhibitor cocktail). The resuspension was incubated in ice for 1 hour with vortex homogenization every 15 min. The homogenate was then spun down at 75000 g at 4° C. for 1 hour (Beckman LE-80K optima ultracentrifuge) to obtain the clarified lysate. The lysate was subjected to pretreatment with 70% saturated ammonium sulfate and ¼^{th} volume of 100% ethanol, followed by vigorous shaking for 2 min (Yakhnin et al, 1998). The treated solution was spun down at 2100 g for 3 min. The upper ethanol phase was collected, and the process was repeated with 1/16^{th} volume of 100% ethanol. The pooled ethanol phases were further treated with ⅓^{rd} volume of 5M NaCl and ¼^{th} volume of 1-butanol, homogenized vigorously for 2 min and spun down at 2100 g for 3 min. The lowermost phase was collected and loaded onto a 7 kDa MWCO zeba spin desalting column (Thermo scientific) and desalted as per manufacturer's recommendations.

The desalted extract was then subjected to hydrophobic interaction chromatography during the capture phase for further purification. The desalted extract was injected into a Toyopearl butyl—650S hydrophobic interaction column (Tosoh bioscience) which was run on a FPLC unit (Pharmacia LKB-FPLC system). The column was equilibriated with 2.3 column volumes of salted buffer (10 mM Tris-HCl, 10 mM EDTA and 50% saturated ammonium sulfate) to a final 20% salt saturation to facilitate binding of GFP onto the resin. This was followed by a column wash with 5.8 column volumes of salted and unsalted buffer mix and then eluted with unsalted buffer (10 mM Tris-HCl, 10 mM EDTA). The GFP fraction was identified based on the peaks observed in the chromatogram and collected. The collected fractions were subjected to a final polishing step by overnight dialysis. After dialysis the purified proteins were lyophilized (labconco lyophilizer) in order to concentrate the finished product and then stored in −20° C.

### Quantification of Purified GFP Fusions

Quantification of GFP-RC101 and GFP-PG1 was done by both western blot and fluorescence-based methods. The lyophilized purified proteins were resuspended in sterile 1X PBS and the total protein was determined by Bradford method. The purified protein was then quantified by SDS-PAGE method by loading denatured protein samples along with commercial GFP standards (Vector labs) onto a 12% SDS gel and then western blotting was done using 1:3000 dilution of mouse Anti-GFP antibody (Millipore) followed by probing with 1:4000 dilution of secondary HRP conjugated Goat-Anti Mouse antibody (Southern biotech).

The purified proteins were also quantified using GFP fluorescence. The protein samples were run on a 12% SDS gel under native conditions. After the run, the gel was placed under a UV lamp and then photographed. The GFP concentration in both western and native fluorescence methods was determined by densitometric analysis using Image J software with commercial GFP standards in order to obtain the standard curve. Purity was determined based on GFP quantitation with respect to total protein values determined in Bradford method.

### Uptake of Purified Tag-Fused GFP Proteins by Human Periodontal Cell Lines

As previously described (Xiao, et al 2016), to determine the uptake of four tags, CTB, PTD, PG1 and RC101, in different human periodontal cell lines, including human periodontal ligament stem cells (HPDLS), maxilla mesenchymal stem cells (MMS), human head and neck squamous cell carcinoma cells (SCC-1), gingiva-derived mesenchymal stromal cells (GMSC), adult gingival keratinocytes (AGK) and osteoblast cells (OBC), briefly, each human cell line cells (2×10⁴) were cultured in 8 well chamber slides (Nunc) at 37° C. overnight, followed by incubation with purified GFP-fused tags: CTB-GFP (8.8 μg), PTD-GFP (13 μg), GFP-PG1 (1.2 μg) and GFP-RC101 (17.3 μg) in 100 μl PBS supplemented 1% FBS at 37° C. for 1 hour. After fixing with 2% paraformaldehyde at RT for 10 min and washing with PBS for three times, all cells were stained with antifade mounting medium with DAPI (Vector laboratories, Inc). For negative control, cells were incubated with commercial GFP (2 μg) in PBS with 1% FBS at 37° C. for 1 hour. All fixed cells were imaged using confocal microscopy. The images were observed under 100× objective, and at least 10-15 GFP-positive cells were recorded for each cell line in three independent analyses.

### Evaluation of Antibacterial Activity

The killing kinetics of AMPs (Gfp-PG1 and Gfp-RC101) against S. mutans were analyzed by time-lapse killing assay. S. mutans were grown to log phase and diluted to 10⁵ CFU/ml in growth medium. GFP-PG1 and GFP-RC101 were added to S. mutans suspensions at concentrations of 0 to 10 μg/ml and 0 to 80 μg/ml, respectively. At 0, 1, 2, 4, 8 and 24 h, samples were taken and serially diluted in 0.89% NaCl, then spread on agar plates and colonies were counted after 48 h. Absorbance at 600 nm was also checked at each time point. S. gordonii, A. naeslundii and C. albicans suspensions were mixed with Gfp-PG1 at concentration of 10 μg/ml, and at 0, 1 and 2 h, aliquots were taken out for enumeration of CFU.

The effects of AMP on the viability of S. mutans cells were also assessed by time-lapsed measurements. S. mutans were grown to log phase and harvested by centrifugation (5500 g, 10 min) and the pellet was washed once with sodium phosphate-buffered saline (PBS) (pH 7.2), re-suspended in PBS and adjusted to a final concentration of 1×10⁵ CFU/ml. GFP-PG1 was added to S. mutans suspensions at concentrations of 10 μg/ml and 2.5 μM propidium iodide-PI (Molecular Probe Inc., Eugene, Oreg., USA) was added for labeling dead cells. 5 μl of mixtures were loaded on an agarose pad for confocal imaging. Confocal images were acquired using Leica SP5-FLIM inverted single photon laser scanning microscope with a 100X (numerical aperture, 1.4) Oil immersion objective. The excitation wavelengths were 488 nm and 543 nm for GFP and PI, respectively. The emission filter for GFP was a 495/540 OlyMPFC1 filter, while PI was a 598/628 OlyMPFC2 filter. For the time-lapse series, images in the same field of view were taken at 0, 10, 30, and 60 min and created by ImageJ 1.44 (on the world wide web at rsbweb.nih.gov/ij/download.html).

Morphological observations of S. mutans treated with AMP were also examined by scanning electron microcopy (SEM). S. mutans were grown to log phase and diluted to 10⁵ CFU/ml in PBS. Bacteria suspension was mixed with GFP-PG1 (final concentration of 10 μg/ml) for 1 h at 37° C. After treatment, the bacteria were collected by filtration using 0.4 μm Millipore filters. The deposits were fixed in 2.5% glutaraldehyde and 2.0% paraformaldehyde in 0.1 M cacodylate buffer (pH 7.4) for 1 hour at room temperature and processed for SEM (Quanta FEG 250, FEI, Hillsboro, Oreg.) observation. Untreated or bacteria treated with buffer only served as controls.

### Evaluation of Anti-Biofilm Activity

A well-characterized EPS-matrix producing oral pathogen, S. mutans UA159, and an opportunistic fungal pathogen, C. albicans SC5314 were used to form single- or mixed-speces biofilms on saliva-coated hydroxyapatite disc surfaces. Briefly, hydroxyapatite discs (1.25 cm in diameter, surface area of 2.7±0.2 cm², Clarkson, Chromatography Products, Inc., South Williamsport, Pa.) were coated with filter-sterilized, clarified human whole saliva (sHA) [Xiao J et alo, 2012]. S. mutans was grown in UFTYE medium with 1% (w/v) glucose to mid-exponential phase (37° C., 5% CO₂). Each sHA disc was inoculated with 10⁵ CFU of actively growing S. mutans cells per ml in UFTYE medium containing 1% (w/v) sucrose, and inoculated at 37° C. and 5% CO₂ for 19 h. Before inoculum, the sHA discs were topically treated with GFP-PG1 solution (10 ug) for 30 min. The inhibition effect of GFP-PG1 treatment on 3D biofilm architectures were observed via confocal imaging. Briefly, EPS was labeled using 2.5 μM Alexa Fluor 647-labeled dextran conjugate (10 kDa; 647/668 nm; Molecular Probes Inc.), while the bacteria and fungal cells were stained with 2.5 μM SYTO9 (485/498 nm; Molecular Probes Inc.) and Concanavalin A-tetramethyl rhodamine conjugate (Molecular Probes). The imaging was performed using Leica SP5 microscope with 20X (numerical aperture, 1.00) water immersion objective. The excitation wavelength was 780 nm, and the emission wavelength filter for SYTO 9 was a 495/540 OlyMPFEC1 filter, while the filter for Alexa Fluor 647 was a HQ655/40M-2P filter. The confocal image series were generated by optical sectioning at each selected positions and the step size of z-series scanning was 2 μm. Amira 5.4.1 software (Visage Imaging, San Diego, Calif., USA) was used to create 3D renderings of biofilm architecture [Xiao J et al. 2012, Koo H et al. 2010].

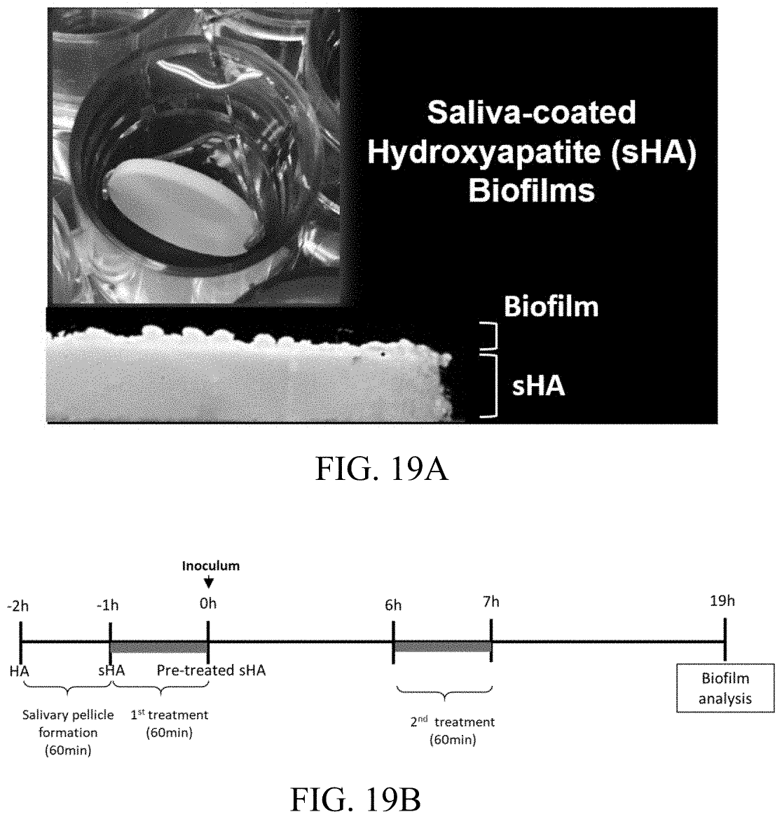

To examine the effects of the PG1 on biofilms formed with S. mutans for 19 h on sHA discs, we examined the 3D architecture of the EPS-matrix and in situ cell viability using time-lapse confocal microscopy following biofilms incubation with 1) Control, 2) EPS-degrading enzymes only, 3) PG1 only, or 4) PG1 and EPS-degrading enzymes for up to 60 minutes. The EPS-degrading enzymes used here were dextranase and mutanase, which were capable of digesting the EPS derived from S. mutans by hydrolyzing α-1,6 glucosidic linkages and α-1,3 glucosidic linkages [Hayacibara et al. 2004]. Dextranase produced from Penicillium sp. was commercially purchased from Sigma (St. Louis, Mo.) and mutanase produced from Trichoderma harzianum was kindly provided by Dr. William H. Bowen (Center for Oral Biology, University of Rochester Medical Center). Dextranase and mutanase were mixed at ratio of 5:1 before applying to biofilms [Mitsue F. Hayacibara et al. 2004]. In addition, lipase, dextranase and mutanase were also tested using similar topical regimen as depicted in FIG. 19B. Alexa Fluor 647-labeled dextran conjugate was used to label the EPS-matrix, while SYTO 9 (or ConA) and PI were used to label live cells and dead cells. Biofilms were examined using confocal fluorescence imaging at 0, 10 30 and 60 min, and subjected to AMIRA/COMSTAT/ImageJ analysis. The total biomass of EPS matrix, live and dead cells in each series of confocal images was quantified using COMSTAT and ImageJ. The ratio of live to the total bacteria at each time point was calculated, and the survival rate of live cells (relative to live cells at 0 min) was plotted. The initial number of viable cells at time point 0 min was considered to be 100%. The percent-survival rate was determined by comparing to time point 0 min.

### Microbiological Assays

At selected time point (19 h), biofilms were removed, homogenized via sonication and subject to microbiological analyses as detailed previously [Xiao J et al. 2012, Koo H et al. 2010]; our sonication procedure does not kill bacteria cells while providing optimum dispersal and maximum recoverable counts. Aliquots of biofilm suspensions were serially diluted and plated on blood agar plates using an automated Eddy Jet Spiral Plater (IUL, SA, Barcelona, Spain). Meanwhile, propidium monoazide (PMA) combined with quantitative PCR (PMA-qPCR) was used for analysis of S. mutans cell viability as describe Klein M I et al. [Klein M I et al. 2012]. The combination of PMA and qPCR will quantify only the cells with intact membrane (i.e. viable cells) because the PMA cross-linked to DNA of dead cells and extracellular DNA modifies the DNA and inhibits the PCR amplification of the extracted DNA. Briefly, biofilm pellets were resuspended with 500 μl TE (50 mM Tris, 10 mM EDTA, pH 8.0). Using a pipette, the biofilm suspensions were transferred to 1.5 ml microcentrifuge tubes; then mixed with PMA. 1.5 μl PMA (20 mM in 20% dimethyl sulfoxide; Biotium, Hayward, Calif.) was added to the biofilm suspensions. The tubes were incubated in the dark for 5 min, at room temperature, with occasional mixing. Next, the samples were exposed to light for 3 min (600-W halogen light source). After photo-induced cross-linking, the biofilm suspensions were centrifuged (13,000 g/10 min/4° C.) and the supernatant was discarded. The pellet was resuspended with 100 μl TE, following by incubation with 10.9 μl lysozyme (100 mg/ml stock) and 5 μl mutanylysin (5 U/μl stock) (37° C./30 min). Genomic DNA was then isolated using the MasterPure DNA purification kit (Epicenter Technologies, Madison, Wis.). Ten pictograms of genomic DNA per sample and negative controls (without DNA) were amplified by MyiQ real-time PCR detection system with iQ SYBR Green supermix (Bio-Rad Laboratories Inc., CA) and S. mutans specific primer (16S rRNA) [Klein M I et al 2010].

### Statistical Analysis

Data are presented as the mean±standard deviation (SD). All the assays were performed in duplicate in at least two distinct experiments. Pair-wise comparisons were made between test and control using Student's t-test. The chosen level of significance for all statistical tests in present study was P<0.05.

The following examples are provided to illustrate certain embodiments of the invention. They are not intended to limit the invention in any way.

### Example I

### Creation and Characterization of Transplastomic Lines

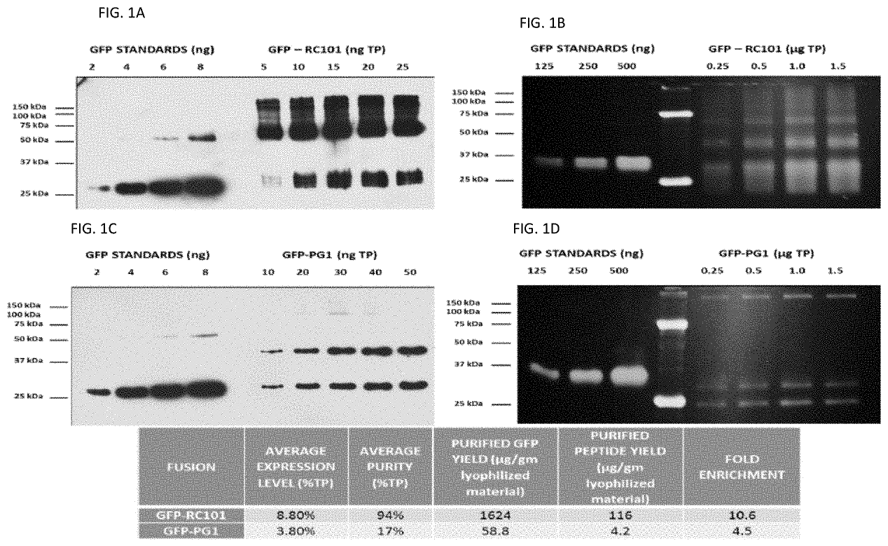

All fusion tags (CTB, PTD, protegrin, retrocyclin) were fused to the green fluorescent protein (smGFP) at N-terminus to evaluate their efficiency and specificity. Fusion constructs encoding these fusion proteins were cloned into chloroplast transformation vectors which were then used to transform plants of interest as described in U.S. patent application Ser. No. 13/101,389 which is incorporated herein by reference. To create plants expressing GFP fusion proteins, tobacco chloroplasts were transformed using biolistic particle delivery system. As seen in the FIG. 1B, each tag-fused GFP is driven by identical regulatory sequences—the psbA promoter and 5′ UTR regulated by light and the transcribed mRNA is stabilized by 3′ psbA UTR. The psbA gene is the most highly expressed chloroplast gene and therefore psbA regulatory sequences are used for transgene expression in our lab [7, 34]. To facilitate the integration of the expression cassette into chloroplast genome, two flanking sequences, isoleucyl-tRNA synthetase (trnI) and alanyl-tRNA synthetase (trnA) genes, flank the expression cassette, which are identical to the native chloroplast genome sequence. The emerging shoots from selection medium were investigated for specific integration of the transgene cassette at the trnI and trnA spacer region and then transformation of all chloroplast genomes in each plant cell (absence of untransformed wild type chloroplast genomes) was confirmed by Southern blot analysis. Thus, stable integration of all GFP expression cassettes and homoplasmy of chloroplast genome with transgenes were confirmed before extracting fusion proteins. In addition, by visualizing the green fluorescence under UV light, GFP expression of was phenotypically confirmed. Confirmed homoplasmic lines were then transferred and cultivated in an automated greenhouse to increase biomass.

To scale up the biomass of each GFP tagged plant leaf material, each homoplasmic line was grown in a temperature- and humidity-controlled greenhouse. Fully grown mature leaves were harvested in late evenings to maximize the accumulation of GFP fusion proteins driven by light-regulated regulatory sequences. To further increase the content of the fusion proteins on a weight basis, frozen leaves were freeze-dried at −40° C. under vacuum. In addition to the concentration effect of proteins, lyophilization increased shelf life of therapeutic proteins expressed in plants more than one year at room temperature [Daniell et al 2015; 2016]. Therefore, in this study, lyophilized and powdered plant cells expressing GFP-fused tag proteins were used for oral delivery to mice.

Expression and Purification of GFP Fused Antimicrobial Peptides from Transplastomic Tobacco.

Tobacco leaves expressing GFP fused antimicrobial peptides RC101 and PG1 were harvested from greenhouse and subsequently lyophilized for protein extraction and purification. The average expression level of GFP-RC101 was found to be 8.8% of total protein in crude extracts while expression of GFP-PG1 was that of 3.8% of total protein based on densitometry. The difference in expression levels was similar to what was reported previously (Lee et al 2011, Gupta et al, 2015).

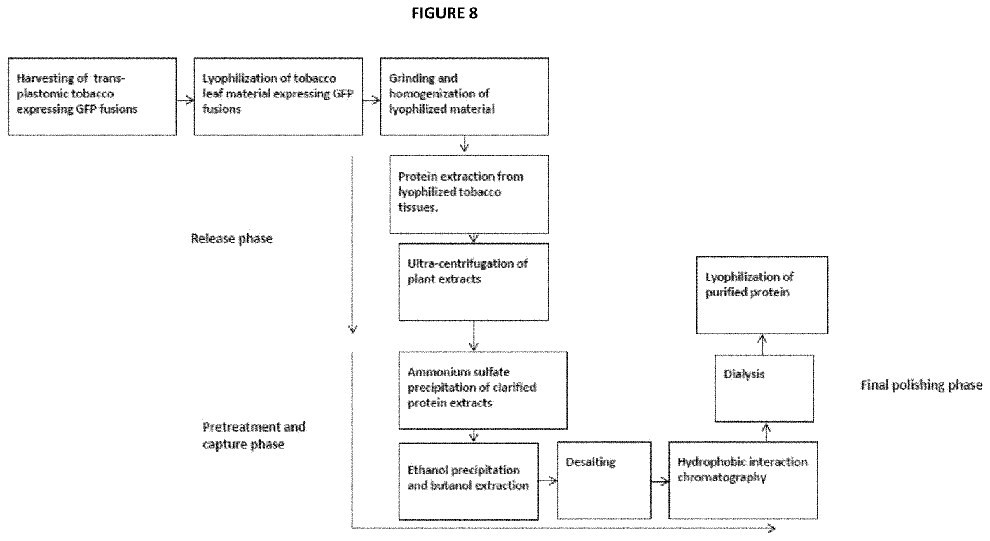

Purification of GFP fused to different antimicrobial peptides (RC101 and PG1) was done in order to test the microbicidal activity against both planktonic and biofilm forming S. mutans. Lyophilized tobacco material expressing different GFP fusions was used for extractions and subsequent downstream processing (See FIG. 8) to obtain the finished purified product which was subsequently quantified to determine concentration of GFP fused peptides. Quantitation of purified GFP-RC101 and GFP-PG1 was done by both western blot and Native GFP fluorescence method where purified GFP-RC101 show 94% average purity with an average yield of 1624 μg of GFP (116 μg of RC101 peptide) per gm of lyophilized leaf material (FIGS. 1A and 1B). In GFP-PG1 both methods (FIGS. 1C and 1D) show 17% average purity with an average yield of 58.8 μg of GFP (4.2 μg PG1 peptide) per gm of lyophilized leaf material. The difference in purity can be attributed to difference in the type of tags fused to GFP as seen in previous studies (Xiao et al 2015, Skosyrev et al 2003). The fold enrichment of purified GFP-RC101 and GFP-PG1 from plant extracts was 10.6 and 4.5 respectively. The western blots also show GFP standards at 27 kDa which corresponds to the monomer fragment along with a 54 kDa GFP dimer with loadings ranging from 6-8 ng of GFP. In GFP-RC101 western blots, 29 kDa and 58 kDa fragments are clearly visible which correspond to the monomer and dimer forms of the fusion (FIG. 1A). This could be attributed to the ability of GFP to form dimers (Ohashi et al, 2007). Western blots of GFP-PG1 (FIG. 1D) clearly show the 29 kDa monomer along with a 40 kDa fragment could be due to mobility shift caused by GFP-PG1 bound to other non-specific plant proteins which could have been co-purified as described previously (Morassuttia et al 2002). Native fluorescence of GFP-RC101 and GFP-PG1 (FIGS. 1B and 1D) show multimeric bands with some of them visible below the 27 kDa GFP standard size which could be because of GFP being fused to cationic peptides causing a electrophoretic mobility shift with each GFP fragment as described in previous studies (Lee et al, 2011).

### Antibacterial Activity of AMPs

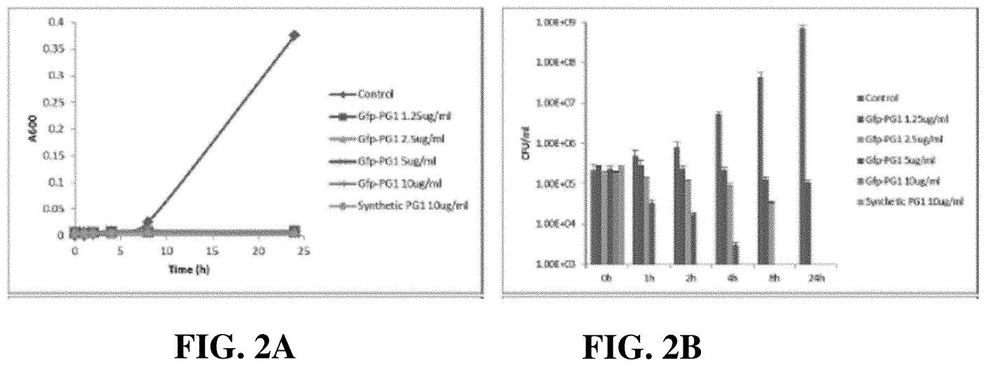

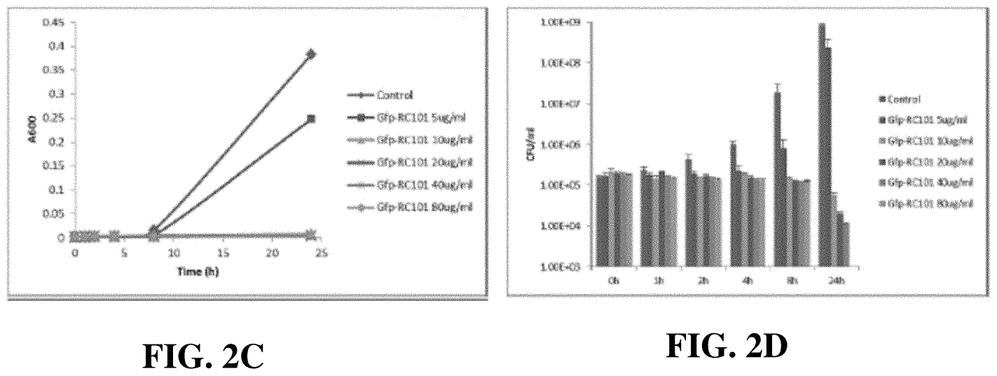

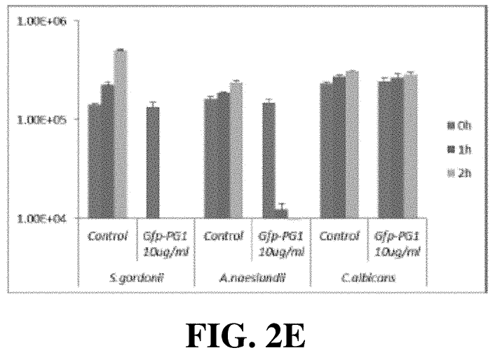

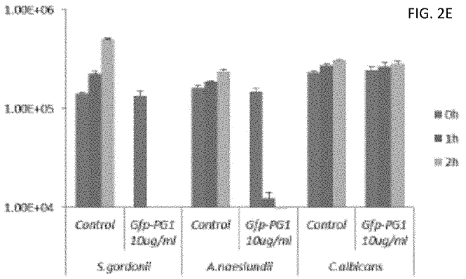

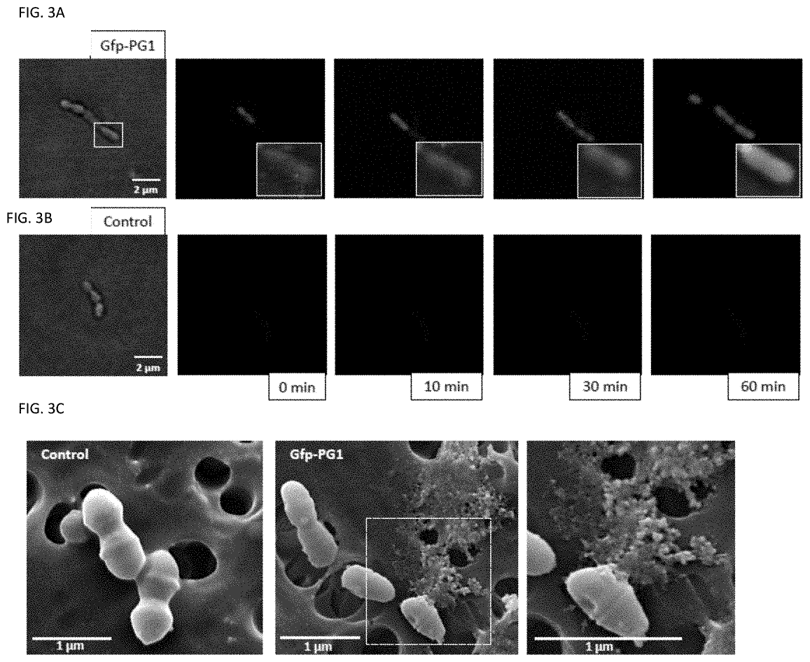

We first examined the antimicrobial activity of GFP-PG1 using dose-response time-kill studies as shown in FIG. 2(A-E). GFP-PG1 displays potent antibacterial activity against Streptococcus mutans, a proven biofilm-forming and caries-causing pathogen, rapidly killing the bacterial cells within 1 h at low concentrations (FIG. 2A). GFP-PG1 also killed the early oral colonizers Streptococcus gordonii and Actinomyces naeslundii, but showed limited antifungal activity against Candida albicans at the concentrations tested (FIG. 2E). Time-lapse confocal imaging shows that S. mutans viability is affected as early as 10 minutes as shown in FIG. 3A relative to the untreated controls (FIG. 3B). SEM imaging revealed disruption of S. mutans membrane surface, causing extrusion of the intracellular content as well as irregular cell morphology, while untreated bacteria showed intact and smooth surfaces without any visible cell lysis or debris (FIG. 3C). Having shown the antimicrobial efficacy of GFP-PG1 against S. mutans, we have examined the potential of this antimicrobial peptide to prevent biofilm formation or disrupt pre-formed biofilms.

### Inhibition of Biofilm Initiation by AMPs

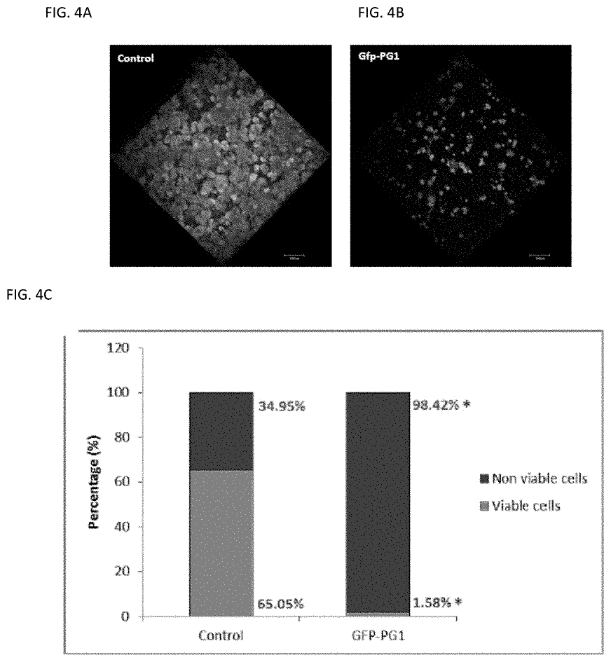

Preventing the formation of pathogenic oral biofilms is challenging because drugs need to exert therapeutic effects following topical applications. To determine whether GFP-PG1 can disrupt the initiation of the biofilm, we treated saliva coated apatitic (sHA) surface (tooth surrogate) with a single topical treatment of GFP-PG1 for 30 min, and then incubated with actively growing S. mutans cells in cariogenic (sucrose-rich) conditions. We observed substantial impairment of biofilm formation by S. mutans with minimal accumulation of EPS-matrix on the GFP-PG1 treated sHA surface (FIGS. 4B and 4C). The few adherent cell clusters were mostly non-viable compared to control (FIG. 4A), demonstrating potent effects of GFP-PG1 on biofilm initiation despite topical, short-term exposure.

Disruption of Pre Formed Biofilm by AMP with or without EPS-Degrading Enzymes

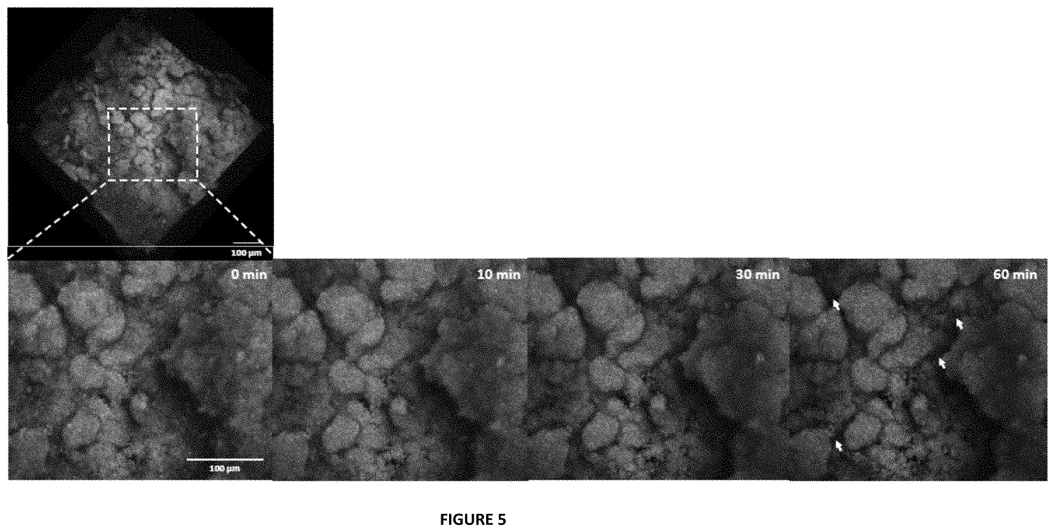

Disruption of formed biofilms on surfaces is challenging. Disruption of cariogenic biofilms is particularly difficult because drugs often fail to reach clusters of pathogenic bacteria (such as S. mutans) because of the surrounding exopolysaccharides (EPS)-rich matrix that enmeshes and protects them [Bowen and Koo, 2011]. EPS-degrading enzymes such as dextranase and mutanase could help digest the matrix of cariogenic biofilms, although they are devoid of antibacterial effects. We first optimized the dextranase and/or mutanase required for EPS-matrix disruption without affecting the cell viability (data not shown). As shown in FIG. 5, the combination of dextranase and mutanase can digest the EPS (in red) and ‘open spaces’ (see arrows) between the bacterial cell clusters (in green) and ‘uncover’ cells (see arrows). Thus, the combination of GFP-PG1 and EPS-degrading enzymes synergistically potentiate the overall antibiofilm effects.

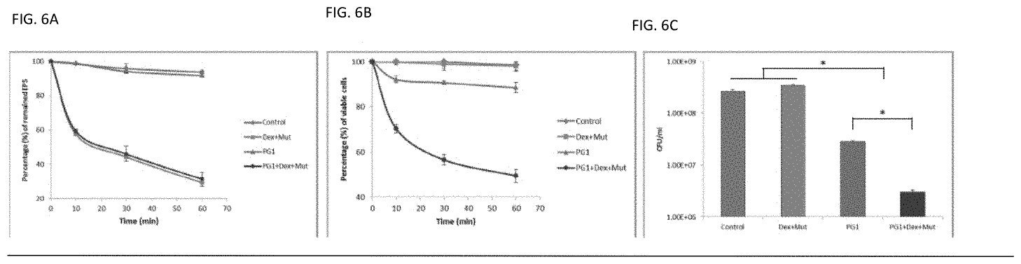

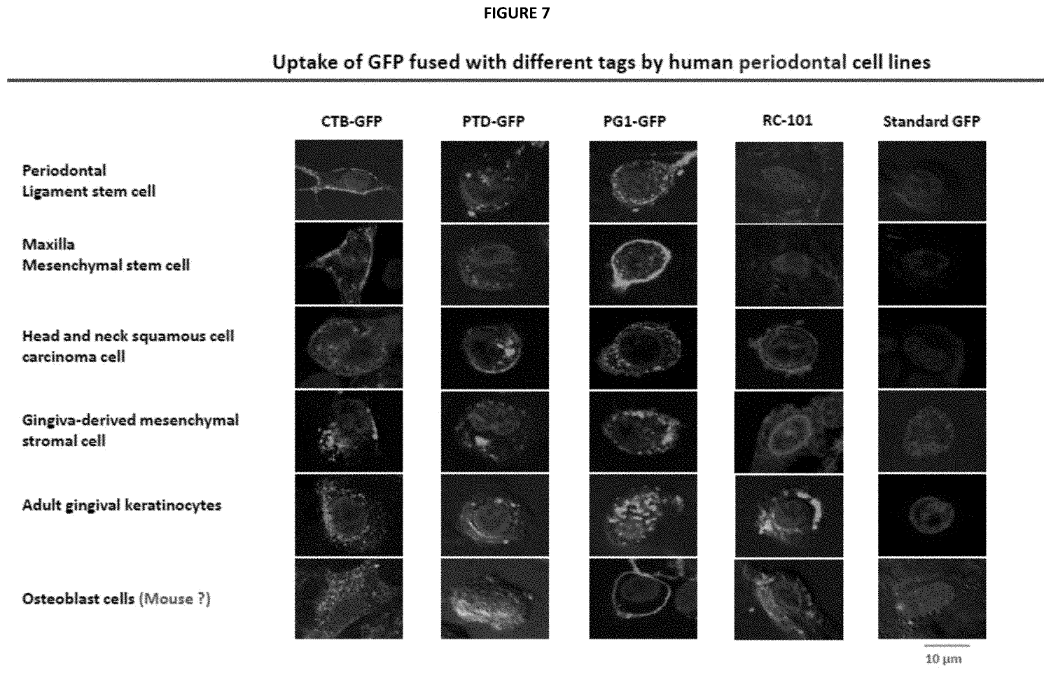

To explore this concept, Streptococcus mutans biofilms were pre-formed on sHA surface, and treated topically with GFP-PG1 and EPS-degrading enzymes (Dex/Mut) either alone or in combination. Time-lapsed confocal imaging and quantitative computational analyses were conducted to analyze EPS-matrix degradation and live/dead bacterial cells within biofilms (FIG. 6A). The enzymes-peptide combination resulted in more than 60% degradation of the EPS-matrix, while increasing the bacterial killing when compared to either GFP-PG or Dex/Mut alone. These findings were further validated via standard culturing assays by determining colony forming units. The antibacterial activity of PG against S. mutans biofilms combined with Dex/Mut was significantly enhanced than either one alone. Topical exposure of Dex/Mut alone showed no effects on biofilm cell viability, whereas GFP-PG-1 alone showed limited killing activity (FIG. 6B). Together, the data demonstrate potential of this combined approach to synergistically enhance antimicrobial efficacy of GFP-PG-1 against established biofilms (FIG. 6C).

Uptake of GFP Fused with Different Tags by Human Periodontal Cells.

Purified GFP fusion proteins when incubated with human cultured cells, including HPDLS, MMS, SCC-1, GMSC, AGK and osteoblast cells (OBC) revealed interesting results. Although only one representive image of each cell line is presented, uptake studies were performed in triplicate and at least 10-15 images were recorded under confocal microscopy. Without a fusion tag, GFP did not enter any tested human cell line. Both CTB-GFP and PTD-GFP effectively penetrated all tested cell types, although their localization patterns differed. Upon incubation with CTB-GFP, GFP signals localized primarily to the periphery of HPDLSC and MMSC, uniformly small cytoplasmic puncta in SSC-1, AGK, OBC and large cytoplasmic foci in GMSC. PTD-GFP was observed as small cytoplasmic foci in MMSC, variably sized cytoplasmic puncta in HPDLSC, GMSC, AGK, OBC and both the cytoplasm and the periphery of SCC-1 cells. PG1-GFP is the most efficient tag in entering all tested human cells because GFP could be localized at tenfold lower concentrations than any other fusion proteins. PG1-GFP showed exclusively cytoplasmic localization in HPDLSC, SCC-1, GMSC and AGK cells and localized to both the periphery and cytosol in MMSC, but it is only localized to the periphery of OBC. RC101-GFP was localized in SCC-1, GMSC, AGK and OBC, but its localization in HPDLSC was negligible and was undetectable in MMSC cells.

### Discussion and Conclusion

The assembly of cariogenic oral biofilms is a prime example of how pathogenic bacteria accumulate on a surface (teeth), as an extracellular EPS matrix develops. Prevention of cariogenic biofilm formation requires disruption of bacterial accumulation on the tooth surface with a topical treatment. Chlorhexidine (CHX) is considered ‘gold standard’ for topical antimicrobial therapy (Flemmig and Beikler 2011; Marsh et al 2011; Caufield et al 2001). CHX effectively suppresses mutans streptococci levels in saliva, but it has adverse side effects including tooth staining and calculus formation, and is not recommended for daily preventive or therapeutic use (Autio-Gold 2008). As an alternative, several antimicrobial peptides (AMP) have been developed and tested against oral bacteria, and have shown potential effects against biofilms (albeit with reduced effects vs planktonic cells) (as reviewed by Silva et al., 2012) Unfortunately, most of these studies tested antibiofilm efficacy using continuous, prolonged biofilm exposure to AMPs (several hours) rather than topical treatment regimen as used clinically. Furthermore, synthetic AMPs are expensive to produce making them unaffordable for dental applications. Here, we show a plant-produced AMP, which demonstrates potent effects in controlling biofilm formation with a single, short-term topical treatment of a tooth-surrogate surface.

Developed cariogenic biofilms are characterized by bacteria embedded in EPS matrix, making biofilm treatment and removal extremely difficult (Paes Leme et al 2006; Koo et al 2013). EPS-rich matrix promotes microbial adhesion, cohesion and protection as well as hindering diffusion (Koo et al 2013; Flemming and Wingender 2010. EPS matrix creates spatial and microenvironmental heterogeneity in biofilms, modulating the growth and protection of pathogens against antimicrobials locally as well as a highly adhesive scaffold that ensures firm attachment of biofilms on tooth surfaces (Flemming and Wingender 2010; Peterson et al. 2015). CHX is far less effective against formed cariogenic biofilms (Hope and Wilson, 2004; Van Strydonck et al 2012; Xiao et al., 2012). The EPS are comprised primarily of a mixture of insoluble (with high content of α1,3 linked glucose) and soluble (mostly α1,6 linked glucose) glucans (Bowen and Koo 2011). Thus, the possibility of using EPS-matrix degrading dextranase or mutanase (from fungi) to disrupt biofilm and prevent dental caries has been explored and included in commercially available over-the-counter mouthwashes (e.g. Biotene PBF). However, topical applications of enzyme alone have generated moderate anti-biofilm/anti-caries effects clinically (Hull 1980), possibly due to lack of antibacterial action and reduced enzymatic activity in the mouth (Balakrishnan et al 2000). Interestingly, a recent in vitro study has shown that a chimeric glucanase comprised of fused dextranase and mutanase is more effective in disrupting plaque-biofilms than either enzyme alone (Jiao et al 2014). However, an approach of combining antimicrobial agents with both EPS-matrix degrading enzymes into a single therapeutic system has not yet been developed, likely due to difficulties associated with cost and formulations. In this study we demonstrate that PG1 together with matrix-degrading enzymes act synergistically and effectively to disrupt cariogenic biofilms. This feasible and efficacious topical antibiofilm approach is capable of simultaneously degrading the biofilm matrix scaffold and killing embedded bacteria using antimicrobial peptides combined with EPS-digesting enzymes.

Retention of high level antimicrobial activity by protegrin along with GFP fusion opens the door for a number of clinical applications to enhance oral health, beyond disruption of biofilms. In addition to biofilm disruption, enhancing wound healing in the gum tissues is an important clinical need. We recently reported that both protegrin and retrocyclin can enter human mast cells and induce degranulation, an important step in the wound healing process (Gupta et al 2015). Therefore, antimicrobial peptides protegrin and retrocyclin play an important role in killing bacteria in biofilms and initiate wound healing through degranulation of mast cells. In addition, it is important to effectively deliver growth hormones or other proteins to enhance cell adhesion, stimulate osteogenesis, angiogenesis, bone regeneration, differentiation of osteoblasts or endothelial cells. Previously identified cell penetrating peptides have several limitations. CTB enters all cell types via the ubiquitous GM1 receptor and this requires pentameric form of CTB. PTD on the other hand does not enter immune cells (Xiao et al 2016).

In this study we tested ability of PG1-GFP or RC101-GFP to enter periodontal and gingival cells. PG1-GFP is the most efficient tag in entering periodontal or gingival human cells because GFP signal could be detected even at ten-fold lower concentrations than any other fusion proteins. Although there were some variations in intracellular localization, PG1-GFP effectively entered HPDLSC, SCC-1, GMSC, AGK, MMSC and OBC. In contrast RC101-GFP entered SCC-1, GMSC, AGK and OBC but its localization in HPDLSC and MMSC cells were poor or undetectable. Therefore, this study has identified a novel role for protegrin in delivering drugs to osteoblasts, periodontal ligament cells, gingival epithelial cells or fibroblasts to enhance oral health. It is feasible to release protein drugs synthesized in plant cells by mechanical grinding and protein drugs bioencapsulated in lyophilized plant cells embedded in chewing gums provides an ideal mode of drug delivery for their slow and sustained release for longer duration. This overcomes a major limitation of current oral rinse formulations—short duration of contact of antimicrobials on the gum/dental surface.

Beyond topical applications, protein drugs fused with protegrin expressed in plant cells can be orally delivered to deeper layers of gum tissues in a non-invasive manner and increase patient compliance. Protein drugs bioencapsulated in plants can be stored for many years at room temperature without losing their efficacy (Su et al 2015; Daniell et al 2016). The high cost of current protein drugs is due to their production in prohibitively expensive fermenters, purification, cold transportation/storage, short shelf life and sterile delivery methods. All these challenges could be eliminated using this novel drug delivery concept to enhance oral health. Recent FDA approval of plant cells for production of protein drugs (Walsh 2014) augurs well for clinical advancement of this novel concept.

### REFERENCES

Agrawal P, Verma D, Daniell H. 2011. Expression of Trichoderma reesei β-mannanase in tobacco chloroplasts and its utilization in lignocellulosic woody biomass hydrolysis. PLoS One. 6:e29302.
Ajdić D, McShan W M, McLaughlin R E, Savić G, Chang J, Carson M B, Primeaux C, Tian R, Kenton S, Jia H, Lin S, Qian Y, Li S, Zhu H, Najar F, Lai H, White J, Roe B A, Ferretti J J. 2002. Genome sequence of Streptococcus mutans UA159, a cariogenic dental pathogen. Proc Natl Acad Sci USA 99:14434-14439.
Autio-Gold J. 2008. The role of chlorhexidine in caries prevention. Oper Dent. 33(6):710-6.
Banas J A, Vickerman M M. 2003. Glucan-binding proteins of the oral streptococci. Crit Rev Oral Biol Med. 14(2):89-99.
Balakrishnan M, Simmonds R S, Tagg J R. 2000. Dental caries is a preventable infectious disease. Aust Dent J. 45:235-45.
Bowen W H. 1972. The effect of dextranase on caries activity in monkeys (macaca irus). Caries Res. 6:75-6.
Bowen W H and Koo H. 2011. Biology of Streptococcus mutans-Derived glucosyltransferases: Role in extracellular matrix formation of cariogenic biofilms. Caries Res 45:69-86.
Burne R A. 1998. Oral streptococci . . . products of their environment. J Dent Res. 77(3):445-52.
Caufield P W, Dasanayake A P, Li Y. 2001. The antimicrobial approach to caries management. J Dent Educ 65:1091-5.
Chen J, Falla T J, Liu H, Hurst M A, Fujii C A, Mosca D A, Embree J R, Loury D J, Radel P A, Cheng Chang C, Gu L, Fiddes J C. 2000. Development of protegrins for the treatment and prevention of oral mucositis: structure-activity relationships of synthetic protegrin analogues. Biopolymers. 55:88-98.
da Silva B R, de Freitas V A, Nascimento-Neto L G, Carneiro V A, Arruda F V, de Aguiar A S, Cavada B S, Teixeira E H. 2012. Antimicrobial peptide control of pathogenic microorganisms of the oral cavity: a review of the literature. Peptides 36: 315-321.
Daniell H, Lin C S, Yu M, Chang W J. 2016. Chloroplast genomes: diversity, evolution and applications in genetic engineering. Genome Biology, in press
DeGray G, Rajasekaran K, Smith F, Sanford J, Daniell H. 2001. Expression of an antimicrobial peptide via the chloroplast genome to control phytopathogenic bacteria and fungi. Plant Physiol. 127(3):852-62.
Dige I, Raarup M K, Nyengaard J R, Kilian M, Nyvad B. 2009. Actinomyces naeslundii in initial dental biofilm formation. Microbiology 155:2116-2126.
Dye B A, Hsu K L, Afful J. 2015. Prevalence and Measurement of Dental Caries in Young Children. Pediatr Dent 37: 200-216.
Flemmig T F, Beikler T. 2011. Control of oral biofilms. Periodontol 2000. 55:9-15.
Flemming H C, Wingender J. 2010. The biofilm matrix. Nat Rev Microbiol. 8:623-33.
Giles F J, Miller C B, Hurd D D, Wingard J R, Fleming T R, Sonis S T, Bradford W Z, Pulliam J G, Anaissie E J, Beveridge R A, Brunvand M M, Martin P J; PROMPT-CT Trial Investigators. 2003. A phase III, randomized, double-blind, placebo-controlled, multinational trial of iseganan for the prevention of oral mucositis in patients receiving stomatotoxic chemotherapy (PROMPT-CT trial). Leuk Lymphoma. 44:1165-72.
Guggenheim B, Regolati B, Schmid R, Mühlemann HR. 1980. Effects of the topical application of mutanase on rat caries. Caries Res. 14:128-35.
Guo L, McLean J S, Yang Y, Eckert R, Kaplan C W, Kyme P, Sheikh O, Varnum B, Lux R, Shi W, He X. 2015. Precision-guided antimicrobial peptide as a targeted modulator of human microbial ecology. Proc Natl Acad Sci USA 112: 7569-7574.
Gupta, Kshitij, Akhil Kotian, Hariharan Subramanian, Henry Daniell, and Hydar Ali. “Activation of Human Mast Cells by Retrocyclin and Protegrin Highlight Their Immunomodulatory and Antimicrobial Properties.” Oncotarget 6, no. 30 (Oct. 6, 2015): 28573-87. doi:10.18632/oncotarget.5611.
Hajishengallis E, Parsaei M, Klein M I, Koo H. 2015. Advances in the microbial etiology and pathogenesis of early childhood caries. Molecular Oral Microbiology doi: 10.1111/omi.12152.
Hall-Stoodley L, Costerton J W, Stoodley P. 2004. Bacterial biofilms: from the natural environment to infectious diseases. Nat Rev Microbiol 2: 95-108.
Hayacibara M F, Koo H, Vacca-Smith A M, Kopec L K, Scott-Anne K, Cury J A, Bowen W H. 2004. The influence of mutanase and dextranase on the production and structure of glucans synthesized by streptococcal glucosyltransferases. Carbohydrate Research 339: 2127-2137.
Hope C K and Wilson M. 2004. Analysis of the effects of chlorhexidine on oral biofilm vitality and structure based on viability profiling and an indicator of membrane integrity. Antimicrob Agents Chemother 48:1461-8.
Hull P S. 1980. Chemical inhibition of plaque. J Clin Periodontol. 7:431-42.
Jin S, Daniell H. 2015. The Engineered Chloroplast Genome Just Got Smarter. Trends Plant Sci 20:622-640
Jiao Y L, Wang S J, Lv M S, Jiao B H, Li W J, Fang Y W, Liu S. 2014. Characterization of a marine-derived dextranase and its application to the prevention of dental caries. J Ind Microbiol Biotechnol. 41:17-26.
Jones C G. 1997. Chlorhexidine: is it still the gold standard? Periodontol 2000 15: 55-62.
Jin S, Kanagaraj A, Verma D, Lange T, Daniell H. 2011. Release of hormones from conjugates: chloroplast expression of β-glucosidase results in elevated phytohormone levels associated with significant increase in biomass and protection from aphids or whiteflies conferred by sucrose esters. Plant Physiol. 155:222-35.
Kassebaum N J, Bernabé E, Dahiya M, Bhandari B, Murray C J, Marcenes W. 2015. Global burden of untreated caries: A systematic review and metaregression. J Dent Res. 94:650-658.
Kohli N, Westerveld D R, Ayache A C, Verma A, Shil P, Prasad T, Zhu P, Chan S L, Li Q, Daniell H. 2014. Oral delivery of bioencapsulated proteins across blood-brain and blood-retinal barriers. Mol Ther. 22:535-46.
Klein M I, Scott-Anne K M, Gregoire S, Rosalen P L, Koo H. Molecular approaches for viable bacterial population and transcriptional analyses in a rodent model of dental caries. Molecular Oral Microbiology 27: 350-361.
Klein M I, DeBaz L, Agidi S, Lee H, Xie G, Lin A H M, Hamaker B R, Lemos J A, Koo H. 2010. Dynamics of Streptococcus mutans transcriptome in response to starch and sucrose during biofilm development. PLoS One: e1347840.
Koo H, Falsetta M L, Klein M I. 2013. The exopolysaccharide matrix: a virulence determinant of cariogenic biofilm. J Dent Res. 92:1065-73.
Koo H, Xiao J, Klein M I, Jeon J G. 2010. Exopolysaccharides produced by Streptococcus mutans glucosyltransferases modulate the establishment of microcolonies within multispecies biofilms. J Bacteriol 192:3024-3032.
Kwon K C, Verma D, Singh N D, Herzog R, Daniell H. 2013. Oral delivery of human biopharmaceuticals, autoantigens and vaccine antigens bioencapsulated in plant cells. Adv Drug Deliv Rev. 65(6):782-99.
Kwon K C, Nityanandam R, New J S, Daniell H. 2013. Oral delivery of bioencapsulated exendin-4 expressed in chloroplasts lowers blood glucose level in mice and stimulates insulin secretion in beta-TC6 cells. Plant Biotechnol J. 11:77-86.
Odd F C. 1988. Candida and candidosis. 2^{nd} ed. Bailliere Tindall, London, United Kingdom.
Peterson B W, He Y, Ren Y, Zerdoum A, Libera M R, Sharma P K, van Winkelhoff A J, Neut D, Stoodley P, van der Mei H C, Busscher H J. 2015. Viscoelasticity of biofilms and their recalcitrance to mechanical and chemical challenges. FEMS Microbiol Rev 39: 234-245.
Lakshmi P S, Verma D, Yang X, Lloyd B, Daniell H. 2013. Low cost tuberculosis vaccine antigens in capsules: expression in chloroplasts, bio-encapsulation, stability and functional evaluation in vitro. PLoS One. 8:e54708.
Lai J R, Huck B R, Weisblum B, Gellman S H. 2002. Design of non-cysteine-containing antimicrobial beta-hairpins: structure-activity relationship studies with linear protegrin-1 analogues. Biochemistry. 41:12835-42.
Lee S B, Li B, Jin S, Daniell H. 2011. Expression and characterization of antimicrobial peptides Retrocyclin-101 and Protegrin-1 in chloroplasts to control viral and bacterial infections. Plant Biotechnol J. 9:100-15.
Ma Z, Wei D, Yan P, Zhu X, Shan A, Bi Z. 2015. Characterization of cell selectivity, physiological stability and endotoxin neutralization capabilities of α-helix-based peptide amphiphiles. Biomaterials. 52:517-30.
Marsh P D, Moter A, Devine D A. 2011. Dental plaque biofilms: communities, conflict and control. Periodontology 2000. 55:16-35.
Morassutti, Carla, Francesca De Amicis, Barbara Skerlavaj, Margherita Zanetti, and Stefano Marchetti. Production of a Recombinant Antimicrobial Peptide in Transgenic Plants Using a Modified VMA Intein Expression System. FEBS Letters 519: 141-46.
Ohashi, Tomoo, Stephane D. Galiacy, Gina Briscoe, and Harold P. Erickson. An Experimental Study of GFP-Based FRET, with Application to Intrinsically Unstructured Proteins. Protein Science: A Publication of the Protein Society 16, no. 7 (July 2007): 1429-38. doi: 10.1110/p s 0.072845607.
Otsuka R, Imai S, Murata T, Nomura Y, Okamoto M, Tsumori H, Kakuta E, Hanada N, Momoi Y. 2015. Application of chimeric glucanase comprising mutanase and dextranase for prevention of dental biofilm formation. Microbiol Immunol. 59:28-36.
Paes Leme A F, Koo H, Bellato C M, Bedi G, Cury J A. 2006. The role of sucrose in cariogenic dental biofilm formation-new insight. J Dent Res. 85:878-87.
Sassi A B, Bunge K E, Hood B L, Conrads T P, Cole A M, Gupta P, Rohan L C. 2011. Preformulation and stability in biological fluids of the retrocyclin RC-101, a potential anti-HIV topical microbicide. AIDS Res Ther. 8:27.
Sassi A B, Cost M R, Cole A L, Cole A M, Patton D L, Gupta P, Rohan L C. 2011. Formulation development of retrocyclin 1 analog RC-101 as an anti-HIV vaginal microbicide product. Antimicrob Agents Chemother. 55:2282-9.
Skosyrev, Vitaly S., Natalja V. Rudenko, Alexander V. Yakhnin, Vasily E. Zagranichny, Lubov I. Popova, Mikhail V. Zakharov, Andrey Yu Gorokhovatsky, and Leonid M. Vinokurov. EGFP as a Fusion Partner for the Expression and Organic Extraction of Small Polypeptides. Protein Expression and Purification 27, no. 1 (January 2003): 55-62.
Trotti A, Garden A, Warde P, Symonds P, Langer C, Redman R, Pajak T F, Fleming T R, Henke M, Bourhis J, Rosenthal D I, Junor E, Cmelak A, Sheehan F, Pulliam J, Devitt-Risse P, Fuchs H, Chambers M, O'Sullivan B, Ang K K. 2004. A multinational, randomized phase III trial of iseganan HCl oral solution for reducing the severity of oral mucositis in patients receiving radiotherapy for head-and-neck malignancy. Int J Radiat Oncol Biol Phys. 58:674-81.
Van Strydonck D A, Slot D E, Van der Velden U, Van der Weijden F. 2012. Effect of a chlorhexidine mouthrinse on plaque, gingival inflammation and staining in gingivitis patients: a systematic review. J Clin Periodontol. 39:1042-55.
Verma D, Kanagaraj A, Jin S, Kolattukudy P E, Daniell H. 2010. Chloroplast-derived enzyme cocktails hydrolyse lignocellulosic biomass and release fermentable sugars. Plant Biotechnol J. 8:332-50.
Walsh G. 2014. Biopharmaceutical benchmarks 2014. Nat Biotechnol. 32:992-1000.
Wang W, Cole A M, Hong T, Waring A J, Lehrer R I. 2003. Retrocyclin, an antiretroviral theta-defensin, is a lectin. J Immunol. 170(9):4708-16.
Xiao, Yuhong, Kwang-Chul Kwon, Brad E. Hoffman, Aditya Kamesh, Noah T. Jones, Roland W. Herzog, and Henry Daniell. Low Cost Delivery of Proteins Bioencapsulated in Plant Cells to Human Non-Immune or Immune Modulatory Cells. Biomaterials 80 (February 2016): 68-79. doi:10.1016/j.biomaterials 0.2015.11.051.
Xiao, J. et al. 2012. The exopolysaccharide matrix modulates the interaction between 3D architecture and virulence of a mixed-species oral biofilm. PLoS Pathog 8, e1002623.
Yakhnin, A. V., L. M. Vinokurov, A. K. Surin, and Y. B. Alakhov. Green Fluorescent Protein Purification by Organic Extraction. Protein Expression and Purification 14, no. 3 (December 1998): 382-86. doi:10.1006/prep.1998.0981.

### Example II

### Creation of Chloroplast Vectors Expressing AMP, Biofilm Degrading Enymes and Fusion Proteins Thereof

Effective treatment of biofilm-associated infections is problematic as antimicrobials often fail to reach clusters of microbes present within the surrounding extracellular matrix that enmeshes and protects them. Furthermore, development of novel therapies against biofilm-related oral diseases and maintenance of oral health needs to be cost-effective and readily accessible.

To ensure a continued supply of reagents, dextranase/mutanase and protegrin/retrocyclin are expressed independently and as fusion proteins in tobacco and other plant chloroplasts, such as lettuce. Proteins will be produced and used in low-cost purification strategies. Tobacco plants produce a million seeds, and thus, it is feasible to scale up production easily. Each acre of tobacco will produce up to 40 metric tons of biomass, facilitating low-cost, large-scale production of AMP, enzymes and fusion constructs encoding the same. In another approach, the proteins are produced in an edible plant such as lettuce.

Several dextranases (Dex) and mutanases (Mut) have been isolated from fungi and bacteria and characterized for their enzymatic activity. Optimal dextranase and mutanase enzymes should have enzymatic properties suitable for human oral environment. Based on short duration of oral treatments, strong binding/retention property to plaque-biofilms and catalytic activity to both types of EPS (dextrans and mutans) are highly desirable. The enzymes added in commercial dextranase-containing mouthwashes (e.g. Biotene) are largely derived from fungi (Penicillium sp. and Chaetomium erraticum). However, fungal dextranases show higher temperature optima (50-60° C.) than bacterial dextranases (35-40° C.). Furthermore, bacterial dextranases are more stable and effective at oral temperature (˜37° C.) and are suitable for dental caries-prevention. Recently, a dextranase from Arthrobacter sp strain Arth410 showed superior dextran degradation properties at optimal temperatures (35-45° C.) and pH values (pH 5-7) found in mouth and in cariogenic biofilms when compared to fungal dextranases. In addition, topical applications of bacterial dextranase are more effective in reducing dental caries in vivo than fungal dextranse. Likewise, a bacterial mutanase from Paenibacillus sp. strain RM1 shows that biofilm was effectively degraded by 6 hr incubation even after removal of the mutanase, preceded by first incubation with the biofilms for 3 min. Also, when compared to other microbial species, RM1 mutanase shows enhanced biofilm-degrading property. Notably, fungal enzymes require glycosylation, which precludes their expression in chloroplasts. In addition, immunogenicity of glycoproteins in human system may raise additional regulatory concerns. Therefore, the present invention involves use of bacterial dextranase and mutanase for expression in chloroplasts.

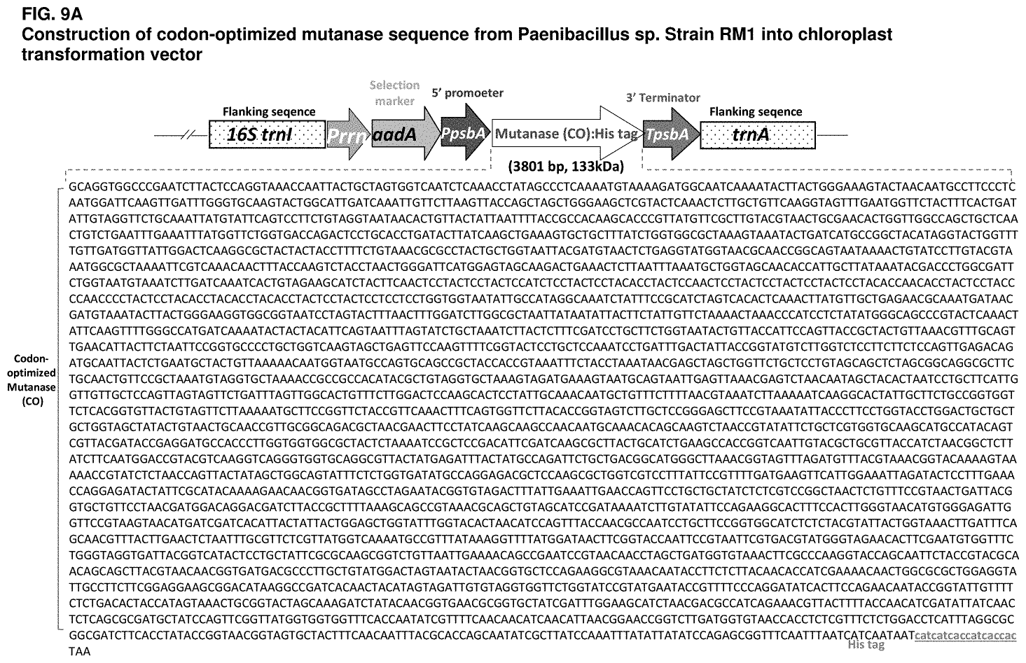

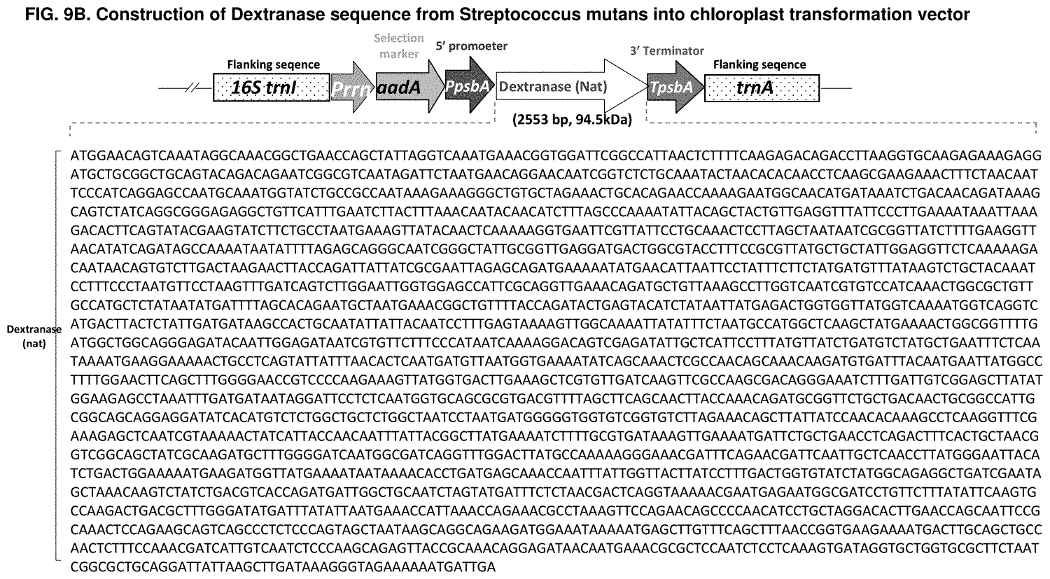

In order to increase the production of Arth410 dextranase and RM1 mutanase protein in chloroplasts, both sequences have been codon optimized for chloroplast expression. See FIGS. 9A and 9B.

### Retrocyclin and Protegrin.

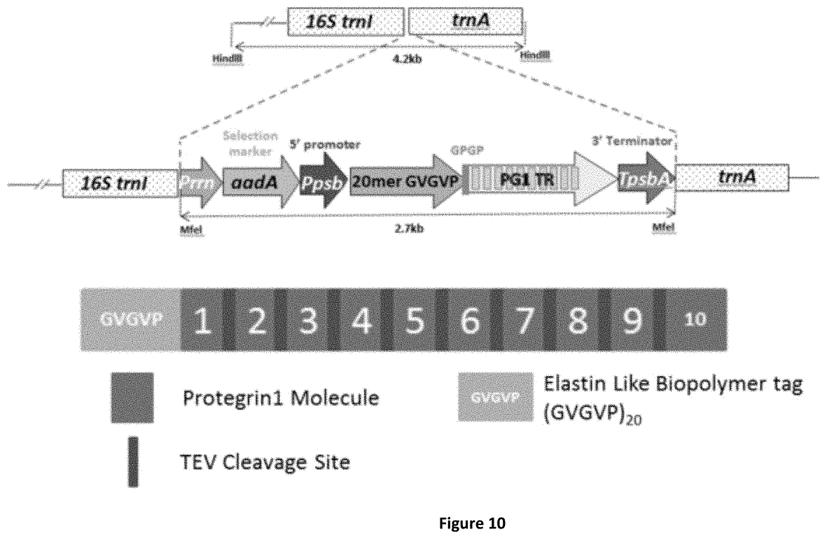

In order to maximize synthesis and reduce toxicity of AMPs, ten tandem repeats of PG1 or RC101, separated by protease cleavage sites as shown in FIG. 10 are employed. For each copy of expressed gene, ten functional copies of PG1 or RC 101 will be made. For this purpose we have chosen the Tobacco Etch Virus (TEV) protease, which has high specificity and a short cleavage site of seven amino acids. Alternatively, furin cleavage sites can also be employed. This vector can also be engineered to include a nucleic acid encoding a biofilm degrading enzyme. The coding region can be expressed under the promoter utilized to express the AMP or can be ligated into the vector operably linked to a second promoter region. The biofilm degrading enzyme coding sequence may also contain TEV protease cleavage sites to facilitate release of the enzyme. This approach provides a safer and cleaner option than chemical cleavage methods. Most importantly, individual PG1 peptides in the fusion protein will not form secondary structures before cleavage, thereby avoiding accumulation of functional peptides which can be lethal to the host production systems. Antimicrobial activity of the cleaved PG1/RC101, biofilm degrading enzymes or fusion proteins thereof can be used to degrade biofilms using the methods disclosed in Example I.

As mentioned above, the sequences encoding the AMP/biofilm degrading enzymes are optionally codon-optimized prior to insertion into chloroplast transformation vectors, such as pLD. Chloroplast transformation relies upon a double homologous recombination event. Therefore, chloroplast vectors comprise homologous regions to the chloroplast genome which flank the expression cassette encoding the heterologous proteins of interest, which facilitate insertion of the transgene cassettes into the intergenic spacer region of the chloroplast genome, without disrupting any functional genes. Although any intergenic spacer region could be used to insert transgenes, the most commonly used site of transgene integration is the transcriptionally active intergenic region between the trnI-trnA genes (in the rrn operon), located within the IR regions of the chloroplast genome (FIG. 10). Because of similar protein synthetic machinery between E. coli and chloroplasts, efficiency of codon-optimization can also be assessed in E. coli and then plants can be created. Both systems could be used for expression of AMPs, biofilm degrading enzymes or fusion proteins thereof, as well as for purification and evaluation of AMPs or enzymatic activities.

### Purification Strategies

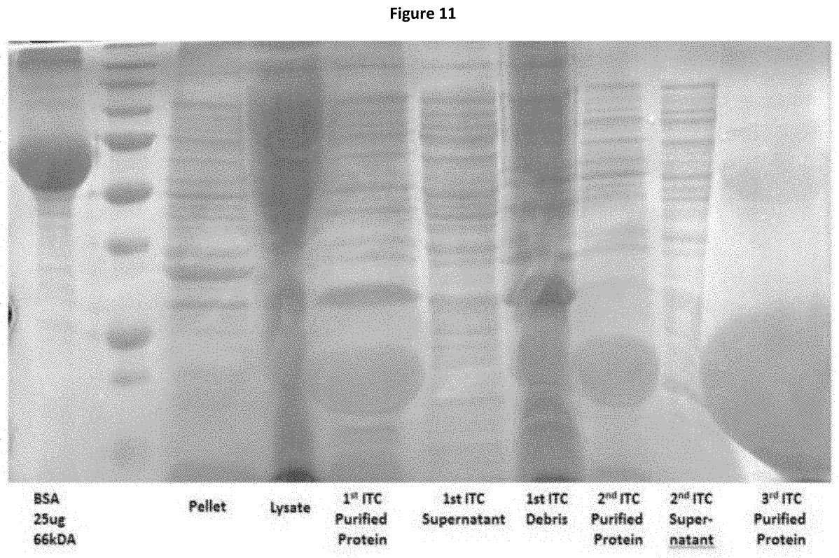

A hydrophobic interaction column (HIC; TOSOH Butyl Toyopearl 650m) can be used to purify PG1 fused with Green Florescent Protein (GFP). The GFP selectively binds to the HIC and facilitates Rc101/PG1 to >90% purity. Despite using the expensive HIC chromatography method, recovery is very poor (<20%). To address this problem and enhance yield, 10 tandem repeats of PG1 with an elastin like biopolymer (GVGVP (SEQ ID NO: 11); FIG. 10) are engineered into the vector. This biopolymer, has a unique thermal property of precipitating out of solution upon increasing temperature above its inverse transition temperature (Tt). GVGVP (SEQ ID NO: 11) remains in soluble monomeric state below Tt and form insoluble aggregates above it. This phase transition from soluble to insoluble state is reversible by changing the temperature of the solution and this facilitates protein purification. Subsequently fused protein is re-solubilized by cooling below Tt and to remove any insoluble contaminants that have co-precipitated as shown in FIG. 11. The process of heating (37° C.) and cooling (4° C.) is known as Inverse Transition Cycling (ITC) and performing 3-5 rounds of ITC results in highly purified proteins (>98% purity, FIG. 11).

In an alternative approach, a signal peptide is fused with dextranase or mutanase for expression in E. coli, where the signal peptide will result in secretion of the enzymes into the extracellular media. In addition, secretory proteins should pass through two membrane systems of E. coli, during which they pass through the periplasmic environment where disulfide isomerases, foldases and chaperones are present. Therefore, correct folding and disulfide bond formation of secretory proteins are facilitated by the enzymes, resulting in enhancement of biological activity of proteins (ideal for AMPs). Another merit of this production strategy is the low level of proteolytic activity in the culture medium which serves to enhance the stability of the recombinant protein. The signal sequence of the secreted protein is cleaved during the export process, creating an authentic N-terminus to the native protein. There are several molecules useful for translocating proteins to extracellular media, such as TAT, SRP, or SecB-dependent pathways. However, rather than working independently, the different pathways closely interact with each other. Both SRP and SecB-dependent pathways can work together in targeting of a single protein. Also, under Sec-deficient conditions, translocation of Sec pathway substrates can be rescued by TAT systems.

Among numerous signal sequences, outer membrane protein A (OmpA) and Seq X (derived from lac Z) signal peptide demonstrate superior export functions and are capable of exporting fused protein into extracellular medium at up to 4 g/L and 1 g/L, respectively. Therefore, these signal sequences are used for efficient exporting of Arth 410 Dex and RM1 Mut to extracellular milieu. Accumulation of the dextranase and mutanase exported into media will be determined by protein quantitation and enzyme assays.

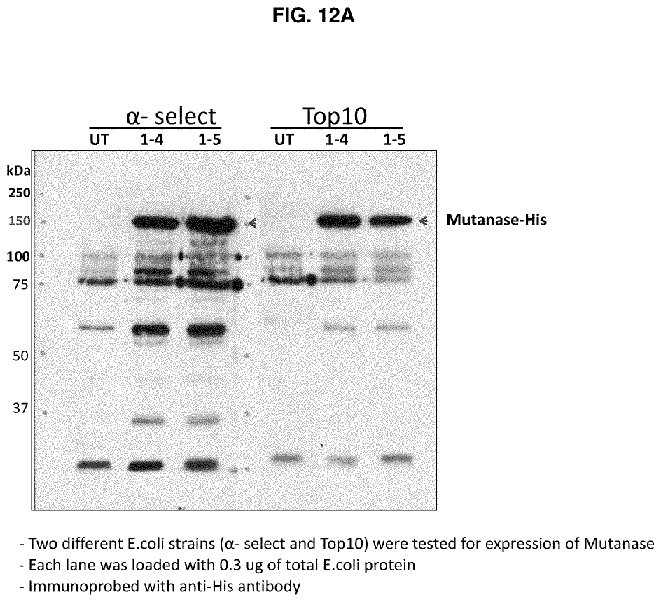

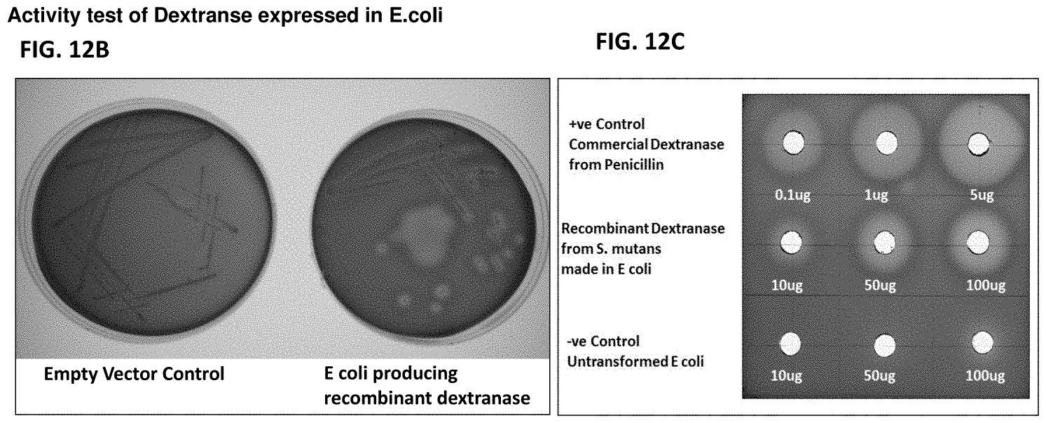

Successful expression of these proteins in E. coli has been achieved. See Western blot results shown in FIG. 12. Chloroplast vectors harboring these sequences will be bombarded into tobacco or lettuce leaves to create plants capable of large scale production of extranase/mutanase/AMP proteins. After harvesting large scale biomass, leaves will be lyophilized and stored at room temperature. In another approach, clinically-proven anti-caries compounds such as (fluoride 250 ppm) and a broad-spectrum bactericidal, chlorhexidine 0.12% can be included to assess whether these agents increase efficacy.

The AMP-enzyme combination effectively disrupts cariogenic biofilm formation and the onset of cavitation in vivo. Furthermore, AMP-enzyme fusion protein appears to be superior to current chemical modalities for antimicrobial therapy and caries prevention.

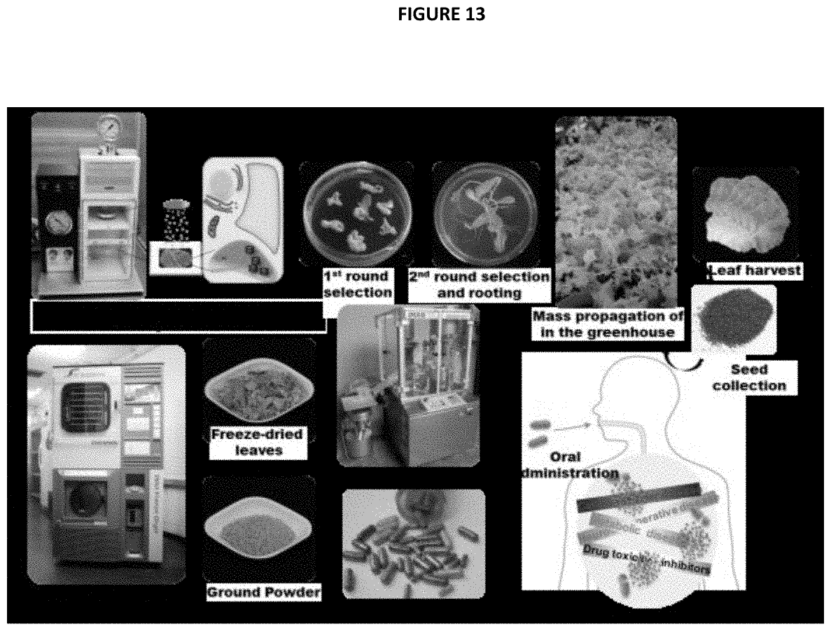

As mentioned previously, effective AMP-enzyme (independently or in combination) can be expressed in lettuce chloroplasts under the control of endogenous lettuce regulatory elements, for large scale GLP production and stability assessment. A key advantage is the lower production cost by elimination of prohibitively expensive purification processes. Freeze-dried leaf material expressing AMP/enzymes can be stored at ambient temperatures for several months or years while maintaining their integrity and functionality. See FIG. 13. In addition to long-term storage, increase of protein drug concentration and decrease of microbial contamination are other advantages. Lettuce leaves, after lyophilization showed 20-25 fold increase in protein drug concentration when compared to fresh leaves, thereby reducing the amount of materials used for oral or topical delivery. Following lyophilization, the plant material can be incorporated into a chewing gum to deliver the biofilm degrading compositions contained therein.

The steps for producing the AMP/enzymes or fusions thereof are shown in FIG. 12. The lettuce chloroplast vectors useful for expressing the proteins of the invention have been previously described in U.S. patent application Ser. No. 12/059,376, which is incorporated herein by reference. Expression levels of up to 70% of total protein in case of therapeutic proteins like proinsulin in lettuce chloroplasts can be achieved using this system.

AMP-enzyme(s) expressed in the edible plants are preferably orally delivered (topically) when used for treatment of oral diseases and the prevention and inhibition of dental carie formation. For enhanced lysis of plant cells within the oral cavity, AMP/enzyme expressing plant cells are optionally mixed with plant cells expressing cell wall degrading enzymes, described in U.S. patent application Ser. No. 12/396,382, also incorporated herein by reference.

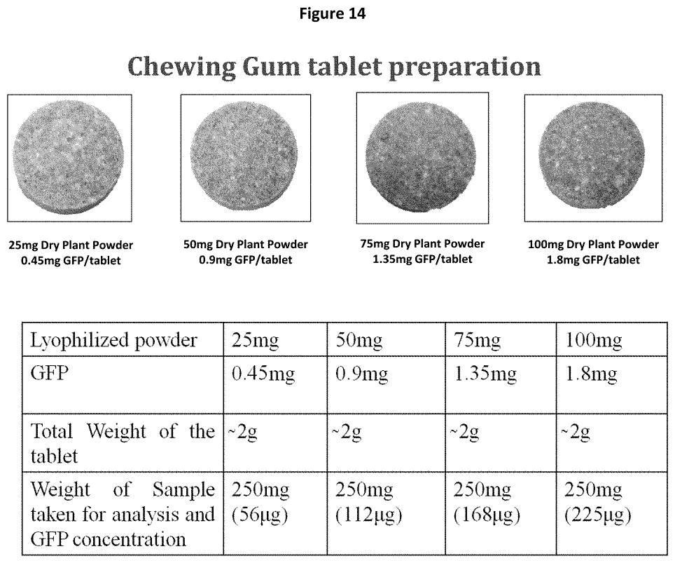

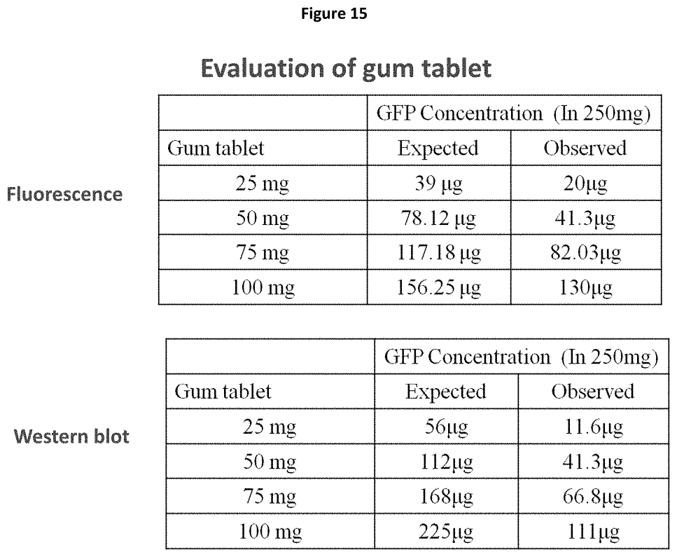

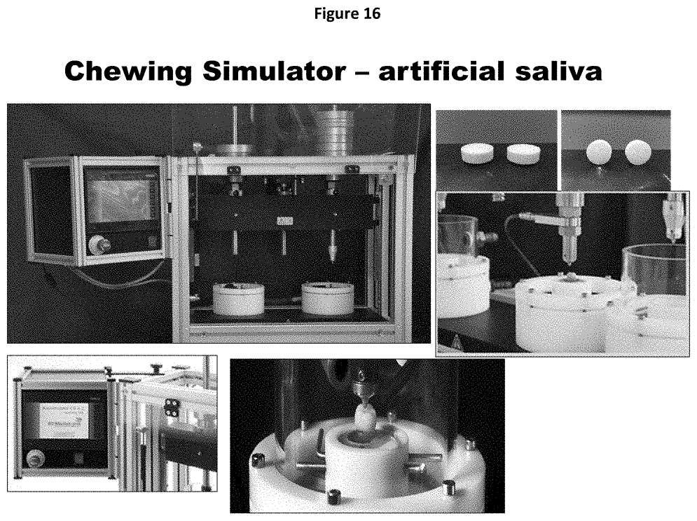

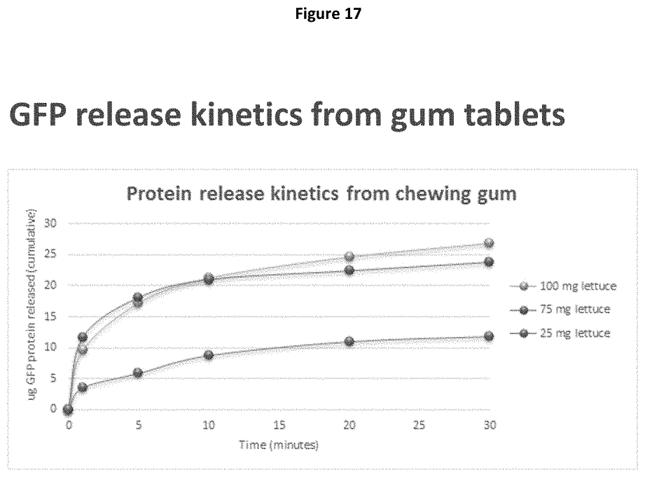

Chewing gum tablet preparation is shown in FIG. 14. Using GFP as an example of the protein of interest, this data shows the amounts of GFP that can be incorporated into a chewing gum tablet. GFP levels were assessed both via fluorescence and by western blot. The results are shown in FIG. 15. The present inventors employed the chewing simulator shown in FIG. 16 and artificial saliva to assess GFP release kinetics from the gum tablets comprising GFP. FIG. 17 shows a graph illustrating the release kinetics over time from gum tablets comprising different amounts of GFP present in recombinant lettuce.

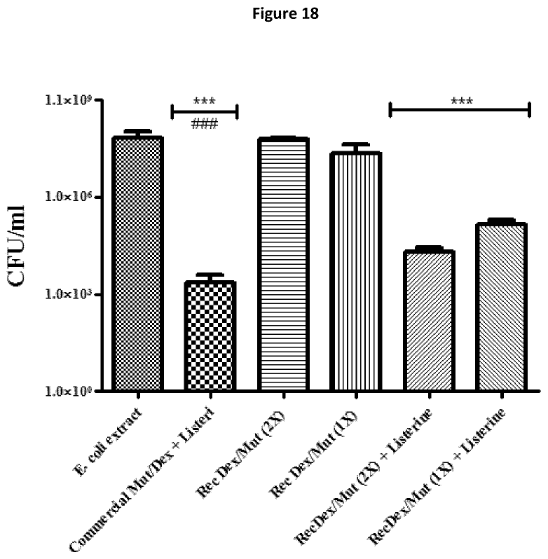

It is clear from these data that gum tablets comprising the AMP-enzyme fusion proteins of the invention will deliver the active material for a suitable time period to achieve bacterial kill and plaque or biofilm degradation. However, oral rinses such as Listerine® (i.e., 0.064% thymol, 0.06% methyl salicylate, 0.042% menthol, 0.092% eucalyptol, ethanol, water, benzoic acid, poloxamer 407, sodium benzoate and caramel) can also be employed to deliver the AMP-enzyme fusion proteins or combinations of the invention. FIG. 18 demonstrates that crude extracts comprising the enzymes of the invention mixed with Listerine® are as effective as commercially produced and purified enzymes that are quite costly to prepare. The data reveal that the dual-enzyme at various combinations (both different ratio and amounts) markedly reduced the biomass of S. mutans biofilm, in a dose-dependent manner. Among different combinations, 25U Dex and 5U Mut (5:1, Dex:Mut ratio) was the most effective, resulting on more than 80% of the total biomass degradation within 120 minutes. Further experiments confirmed that 5:1 Dex/Mut activity ratio displayed the highest effectiveness for both EPS degradation and bacterial killing by Listerine®. Excitingly, the dual-enzyme pre-treatment dramatically enhanced the efficacy of Listerine®-mediated bacterial killing (>3 log reduction vs vehicle pre-treatment and Listerine®). The inclusion of a third enzyme further enhanced the overall anti-biofilm activity. Furthermore, results from the mixed-species model indicated that the dual-enzyme combination was capable of not only enhancing the overall antibacterial activity, but also inducing targeted reduction of S. mutans dominance (while increasing the proportion of commensal/probiotic S. oralis) when Listerine® was used after enzymes pre-treatment. Accordingly, the enzyme+Listerine® strategy should selectively target the pathogen S. mutans, while increasing the proportion of commensal S. oralis, thereby preventing microecological imbalance within mixed-species biofilm.

AMPS have the ability to stimulate innate immunity and wound healing, in addition to antimicrobial activity. Harnessing this novel mast cell host defense feature of AMPs in addition to their antimicrobial properties expands their clinical applications. Biofilm-associated caries is the most challenging model for development of topical therapeutics. When developed, such topical drug delivery can be easily adapted to other biofilms, as matrix formation hinders drug efficacy in many other biofilm-associated diseases. Matrix is inherent in all biofilms thus the application goes beyond the biofilm in the mouth. The biofilm inhibiting compositions described herein can also be employed in coating stents, artificial joints, implants, valves and other medical devices inserted into the human body for the treatment of disease.

As discussed above, the AMP/enzymes, or leaves expressing the same can be incorporated into a chewing gum for effective topical application of the same for the treatment of oral disease. The compositions may also be incorporated into an oral rinse, such as Listerine®. As mentioned previously, other anti dental carrie agents such as fluoride or CHX may included in such gums or oral rinses.

The references below in Table 2 describe a number of different mutanases from a variety of biological sources. Each of these references incorporated herein by reference.

| | Reference | Year | Mutanase resource |
|---|---|---|---|
| 1 | Otsuka R, Imai S, Murata T, etal. (2014) Application of chimeric glucanase comprising | 2014 | Paenibacillus humicus NA1123 |
| | mutanase and dextranase for prevention of dental biofilm formation. Microbiology and | | |
| | Immunology n/a-n/a | | |
| 2 | Wiater A, Pleszczynska M, Rogalski J, Szajiecka L & Szczodrak J (2013) Purification | 2013 | Trichoderma harzianum CCM F-340 |
| | and properties of an alpha-(1 → 3)-glucanase (EC 3.2.1.84) from Trichoderma | | |
| | harzianum and its use for reduction of artificial dental plaque accumulation. Acta | | |
| | Biochim Po 160: 123-128. | | |
| 3 | Wiater A, Janczarek M, Choma A, Próchniak K, Komaniecka I & Szczodrak J (2013) | 2013 | Trichoderma harzianum CCM F-340 |
| | Water-soluble (1 → 3), (1 → 4)-α-d-glucan from mango as a novel inducer of cariogenic | | |
| | biofilm-degrading enzyme. International Journal of Biological Macromolecules 58: | | |
| | 199-205. | | |
| 4 | Tsumori H, Shimamura A, Sakurai Y & Yamakami K (2012) Combination of Mutanase | 2012 | Paenibacillus humicus |
| | and Dextranase Effectively Suppressed Formation of Insoluble Glucan Biofilm by | | |
| | Cariogenic Streptococci. Interface Oral Health Science 2011, (Sasaki K, Suzuki O & | | |
| | Takahashi N , ed.\hat{ }eds.), p.\hat{ }pp. 215-217. Springer Japan. | | |
| 5 | Xiao J, Klein M I, Falsetta M L, etal. (2012) The Exopolysaccharide Matrix Modulates the | 2012 | Trichoderma harzianum |
| | Interaction between 3D Architecture and Virulence of a Mixed-Species Oral Biofilm. | | |
| | PLoS Pathog 8:e1002623. | | |
| 6 | Tsumori H, Shimamura A, Sakurai Y & Yamakami K (2011) Mutanase of | 2011 | Paenibacillus humicus |
| | <i> Paenibacillus humicus</i> from Fermented Food Has a Potential for Hydrolysis of | | |
| | Biofilms Synthesized by <i> Streptococcus mutans</i>. Journal of Health Science 57: | | |
| | 420-424. | | |
| 7 | Wiater A, Szczodrak J & Pleszczyńska M (2008) Mutanase induction in Trichoderma | 2008 | Trichoderma harzianum |
| | harzianum by cell wall of Laetiporus sulphureus and its application formutan removal | | |
| | from oral biofilms. J Microbiol Biotechnol 18: 1335-1341. | | |
| 8 | Shimotsuura I, Kigawa H, Ohdera M, Kuramitsu H K & Nakashima S (2008) | 2008 | Paenibacillus sp. strain RM1 |
| | Biochemical and Molecular Characterization of a Novel Type of Mutanase from | | |
| | Paenibacillus sp. Strain RM 1: Identification of Its Mutan-Binding Domain, Essential for | | |
| | Degradation of Streptococcus mutans Biofilms. Applied and Environmental | | |
| | Microbiology 74: 2759-2765 | | |
| 9 | Shimotsuura I, Kigawa H, Ohdera M, Kuramitsu H K & Nakashima S (2008) | 2008 | Paenibacillus sp. strain RM1 |
| | Biochemical and Molecular Characterization of a Novel Type of Mutanase from | | |
| | Paenibacillus sp. Strain RM 1: Identification of Its Mutan-Binding Domain, Essential for | | |
| | Degradaion of Streptococcus mutans Biofilms. Applied and Environmental | | |
| | Microbiology 74: 2759-2765. | | |
| 10 | Wiater A, Szczodrak J; Pleszczyska M; Próchniak K (2005) Production and use of | 2005 | Trichoderma harzianum CCM F-340 |
| | mutanase from Trichoderma harzianum for effective degradation of streptococcal | | |
| | mutans. Braz. J. Microbiol. vol 36 no. 2 | | |
| 11 | Hayacibara M F, Koo H, Vacca Smith A M, Kopec L K, Scott-Anne K, Cury J A & Bowen | 2004 | Trichoderma harzianum |
| | W H (2004) The influence of mutanase and dextranase on the production and structure | | |
| | of glucans synthesized by streptococcal glucosyltransferases. Carbohydrate Research | | |
| | 339: 2127-2137 | | |
| 12 | Kopec L K, Vacca Smith A M, Wunder D, Ng-Evans L & Bowen W H (2001) Properties of | 2001 | Trichoderma harzianum |
| | Streptococcus sanguinis glucans formed under various conditions. Caries Res 35: 67- | | |
| | 74. | | |
| 13 | Kopec L K, Vacca-Smith A M & Bowen W H (1997) Structural aspects of glucans formed | 1997 | Trichoderma harzianum CCM F-341 |
| | in solution and on the surface of hydroxyapatite. Glycobiology 7: 929-934. | | |
| 14 | Vacca-Smith A M, Venkitaraman A R, Quivey R G, Jr. & Bowen W H (1996) Interactions of | 1996 | Trichoderma harzianum |
| | streptococcal glucosyltransferases with alpha-amylase and starch on the surface of | | |
| | saliva-coated hydroxyapatite. Arch Oral Biol 41: 291-298 | | |
| 15 | Quivey R G, Jr. & Kriger P S (1993) Raffinose-induced mutanase production from | 1993 | Trichoderma harzianum |
| | Trichoderma harzianum. FEMS Microbiol Lett 112: 307-312. | | |
| 16 | Inoue M ,Yakushiji T, Mizuno J, Yamamoto Y & Tanii S (1990) Inhibition of dental | 1990 | Pseudomonas sp. strain |
| | plaque formation by mouthwash containing an endo-alpha-1,3 glucanase. Clin Prev | | |
| | Dent 12: 10-14. | | |
| 17 | Inoue M, Yakushiji T, Katsuki M, Kudo N & Koga T (1988) Reduction oft he adherence | 1988 | Pseudomonas sp. |
| | of Streptococcus sobrinus insoluble α-d-glucan by endo-(1 → 3)-α-d-glucanase. | | |
| | Carbohydrate Research 182: 277-286. | | |
| 18 | Kelstrup J, Holm-Pedersen P & Poulsen S (1978) Reduction of the formation of dental | 1978 | Trichoderma harzianum |
| | plaque and gingivitis in humans by crude mutanase. European Journal of Oral | | |
| | Sciences 86: 93-102. | | |
| 19 | Kelstrup J, Holm-Pedersen P & Poulsen S (1978) Reduction of the formation of dental | 1978 | Trichoderma harzianum |
| | plaque and gingivitis in humans by crude mutanase. Scand J Dent Res 86: 93-102. | | |
| 20 | Guggenhein B, Regolati B & Mühlemann H R (1972) Caries and Plaque Inhbition by | 1972 | Trichoderma harzianum OMZ 779 |
| | Mutanase in Rats. Caries Research 6: 289-297. | | |

Additional biofilm degrading enzyme encoding sequences useful in the practice of the invention, include without limitation,
I) Paenibacillus humicus NA1123
See also the world wide web at ncbi.nlm.nih.gov/nuccore/AB489092 Genbank AB489092

### Length: 1,146

### Mass (Da): 119,007

Reference: Otsuka R, et al. Microbiol Immunol. 2015 January; 59(1):28-36.
2. The Protein Sequence of Mutanase from Paenibacillus humicus NA1123

>gi|257153265|dbj|BA123187.1| putative mutanase [Paenibacillus humicus]
( SEQ ID NO: 12)
MRIRTKYMNWMLVLVLIAAGFFQAAGPIAPATAAGGANLTLGKTVTASGQSQTYSPDNVKDSNQGTYWE
STNNAFPQWIQVDLGASTSIDQIVLKLPSGWETRTQTLSIQGSANGSTFTNIVGSAGYTFNPSVAGNSV
TINFSAASARYVRLNFTANTGWPAGQLSELEIYGATAPTPTPTPTPTPTPTPTPTPTPTVTPAPSATPT
PTPPAGSNIAVGKSITASSSTQTYVAANANDNNTSTYWEGGSNPSTLTLDFGSNQSITSVVLKLNPASE
WGTRTQTIQVLGADQNAGSFSNLVSAQSYTFNPATGNTVTIPVSATVKRLQLNITANSGAPAGQIAEFQ
VFGTPAPNPDLTITGMSWTPSSPVESGDITLNAVVKNIGTAAAGATTVNFYLNNELAGTAPVGALAAGA
SANVSINAGAKAAATYAVSAKVDESNAVIEQNEGNNSYSNPTNLVVAPVSSSDLVAVTSWSPGTPSQGA
AVAFTVALKNQGTLASAGGAHPVTVVLKNAAGATLQTFTGTYTGSLAAGASANISVGSWTAASGTYTVS
TTVAADGNEIPAKQSNNTSSASLTVYSARGASMPYSRYDTEDAVLGGGAVLRTAPTFDQSLIASEASGQ
KYAALPSNGSSLQWTVRQGQGGAGVTMRFTMPDTSDGMGQNGSLDVYVNGTKAKTVSLTSYYSWQYFSG
DMPADAPGGGRPLFRFDEVHFKLDTALKPGDTIRVQKGGDSLEYGVDFIEIEPIPAAVARPANSVSVTE
YGAVANDGKDDLAAFKAAVTAAVAAGKSLYIPEGTFHLSSMWEIGSATSMIDNFTVTGAGIWYTNIQFT
NPNASGGGISLRIKGKLDFSNIYMNSNLRSRYGQNAVYKGFMDNFGTNSIIHDVWVEHFECGMWVGDYA
HTPAIYASGLVVENSRIRNNLADGINFSQGTSNSTVRNSSIRNNGDDGLAVWTSNTNGAPAGVNNTFSY
NTIENNWRAAAIAFFGGSGHKADHNYIIDCVGGSGIRMNTVFPGYHFQNNTGITFSDTTIINSGTSQDL
YNGERGAIDLEASNDAIKNVTFTNIDIINAQRDGVQIGYGGGFENIVFNNITIDGTGRDGISTSRFSGP
HLGAAIYTYTGNGSATFNNLVTRNIAYAGGNYIQSGFNLTIK

3. Sequence of mRNA from Paenibacillus humicus NA1123

```
>gi|257153264|dbj|AB489092.1| Paenibacillus humicus mut gene for
putative mutanase, complete cds
(SEQ ID NO: 13)
      1 aaaggaggat cgccaaccaa tcatcccagc aaagaaggtg atggcagccc aagaattgaa
    61 agcgctttga atttggaata tacggatttg gccgacctgc tgattcagtc gtattcaagc
  121 gattatgccg cgaaccaatc gaacccgagg aggactataa tgcgtatccg cactaaatat
  181 atgaactgga tgttggtgct cgtcctgatc gccgccggct tcttccaggc tgccggcccc
  241 atcgctcccg ccaccgctgc aggaggcgcg aatctgacgc tcggcaaaac cgtcaccgcc
  301 agcggccagt cgcagacgta cagccccgac aatgtcaagg acagcaatca gggaacttac
  361 tgggaaagca cgaacaacgc cttcccgcag tggatccaag tcgaccttgg cgccagcacg
  421 agcatcgacc agatcgtgct caagcttccg tccggatggg agactcgtac gcaaacgctc
  481 tcgatacagg gcagcgcgaa cggctcgacg ttcacgaaca tcgtcggatc ggccgggtat
  541 acattcaatc catccgtcgc cggcaacagc gtcacgatca acttcagcgc tgccagcgcc
  601 cgctacgtcc gcctgaattt cacggccaat acgggctggc cagcaggcca gctgtcggag
  661 cttgagatct acggagcgac ggcgccaacg cctactccca cgcctactcc aacaccaacg
  721 ccaacgccaa caccaacgcc aacccctaca gtaacccctg cgccttcggc cacgccgact
  781 ccgactcctc cggcaggcag caacatcgcc gtagggaaat cgattacagc ctcttccagc
  841 acgcagacct acgtagctgc aaatgcaaat gacaacaata catccaccta ttgggaggga
  901 ggaagcaacc cgagcacgct gactctcgat ttcggttcca accagagcat cacttccgtc
  961 gtcctcaagc tgaatccggc ttcggaatgg gggactcgca cgcaaacgat ccaagttctt
1021 ggagcggatc agaacgccgg ctccttcagc aatctcgtct ctgcccagtc ctatacgttc
1081 aatcccgcaa ccggcaatac ggtgacgatt ccggtctccg cgacggtcaa gcgcctccag
1141 ctgaacatta cggcgaactc cggcgcccct gccggccaga ttgccgagtt ccaagtgttc
1201 ggcacgccag cgcctaatcc ggacttgacc attaccggca tgtcctggac tccgtcttct
1261 ccggtcgaga gcggcgacat tacgctgaac gccgtcgtca agaacatcgg aactgcagct
1321 gcaggcgcca cgacggtcaa tttctacctg aacaacgaac tcgccggcac cgctccggta
1381 ggcgcgcttg cggcaggagc ttctgcaaat gtatcgatca atgcaggcgc caaagcagcc
1441 gcaacgtatg cggtaagcgc caaagtcgac gagagcaacg ccgtcatcga gcagaatgaa
1501 ggcaacaaca gctactcgaa cccgactaac ctcgtcgtag cgccggtgtc cagctccgac
1561 ctcgtcgccg tgacgtcatg gtcgccgggc acgccgtcgc agggagcggc ggtcgcattt
1621 accgtcgcgc ttaaaaatca gggtacgctg gcttccgccg gcggagccca tcccgtaacc
1681 gtcgttctga aaaacgctgc cggagcgacg ctgcaaacct tcacgggcac ctacacaggt
1741 tccctggcag caggcgcatc cgcgaatatc agcgtgggca gctggacggc agcgagcggc
1801 acctataccg tctcgacgac ggtagccgct gacggcaatg aaattccggc caagcaaagc
1861 aacaatacga gcagcgcgag cctcacggtc tactcggcgc gcggcgccag catgccgtac
1921 agccgttacg acacggagga tgcggtgctc ggcggcggag ctgtcctgag aacggcgccg
1981 acgttcgatc agtcgctcat cgcttccgaa gcatcgggac agaaatacgc cgcacttccg
2041 tccaacggct ccagcctgca gtggaccgtc cgtcaaggcc agggcggtgc aggcgtcacg
2101 atgcgcttca cgatgcccga cacgagcgac ggcatgggcc agaacggctc gctcgacgtc
2161 tatgtcaacg gaaccaaagc caaaacggtg tcgctgacct cttattacag ctggcagtat
2221 ttctccggcg acatgccggc tgacgctccg ggcggcggca ggccgctctt ccgcttcgac
2281 gaagtccact tcaagctgga tacggcgttg aagccgggag acacgatccg cgtccagaag
2341 ggcggtgaca gcctggagta cggcgtcgac ttcatcgaga tcgagccgat tccggcagcg
2401 gttgcccgtc cggccaactc ggtgtccgtc accgaatacg gcgctgtcgc caatgacggc
2461 aaggatgatc tcgccgcctt caaggctgcc gtgaccgcag cggtagcggc cggaaaatcc
2521 ctctacatcc cggaaggcac cttccacctg agcagcatgt gggagatcgg ctcggccacc
2581 agcatgatcg acaacttcac ggtcacgggt gccggcatct ggtatacgaa catccagttc
2641 acgaatccca atgcatcggg cggcggcatc tccctgagaa tcaaaggaaa gcttgatttc
2701 agcaacatct acatgaactc caacctgcgt tcccgttacg ggcagaacgc cgtctacaaa
2761 ggctttatgg acaatttcgg cactaattcg atcatccatg acgtctgggt cgagcatttc
2821 gaatgcggca tgtgggtcgg cgactacgcc catactcctg cgatctatgc gagcgggctc
2881 gtcgtggaaa acagccgcat ccgcaacaat cttgccgacg gcatcaactt ctcgcaggga
2941 acgagcaact cgaccgtccg caacagcagc atccgcaaca acggcgatga cggcctcgcc
3001 gtctggacga gcaacacgaa cggcgctccg gccggcgtga acaacacctt ctcctacaac
3061 acgatcgaga acaactggcg cgcggcggcc atcgccttct tcggcggcag cggccacaag
3121 gctgaccaca actacatcat cgactgtgtc ggcggctccg gcatccggat gaatacggtg
3181 ttcccaggct accacttcca gaacaacacc ggcatcacct tctcggatac gacgatcatc
3241 aacagcggca ccagccagga tctgtacaac ggcgagcgcg gagcgattga tctggaagct
3301 tccaacgacg cgatcaaaaa cgtcaccttc accaacatcg acatcatcaa tgcccagcgc
3361 gacggcgttc agatcggcta tggcggcggc ttcgagaaca tcgtgttcaa caacatcacg
3421 atcgacggca ccggccgcga cgggatatcg acatcccgct tctcgggacc tcatcttggc
3481 gcagccatct atacgtacac gggcaacggc tcggcgacgt tcaacaacct ggtgacccgg
3541 aacatcgcct atgcaggcgg caactacatc cagagcgggt tcaacctgac gatcaaatag
3601 gctgcaaaaa aaaggaagct cctcggagct tccttttttt
```

II) Paenibacillus curdlanolyticus MP-1
1. General Information of Mutanase from Paenibacillus curdlanolyticus MP-1
See the world wide web at ncbi.nlm.nih.gov/nuccore/HQ640944

### Genbank HQ640944; Length: 1,261; Mass (Da): 131,631

Reference: Pleszczyńska M, et al. Protein Expr Purif. 2012 November; 86(1):68-74.
2. The Protein Sequence of Mutanase from Paenibacillus curdlanolyticus MP-1

>gi|315201261|gb|ADT91063.1|alpha-1,3-glucanase
[Paenibacillus curdlanolyticus]
(SEQ ID NO: 14)
MRNKYVTWTLALTMLFSSFFLAVGPNKVVHAAGGTNLALGKNVTASGQSQT
YSPNNVKDSNQSTYWESTNNAFPQWIQVDLGATTSIDQIVLKLPAGWGTRT
QTLAVQGSTDGSSFTNIVGSAGYVFNPAVANNAVTINFSAASTRYVRLNVT
ANTAWPAAQLSEFEIYGAGGTTTPPTTPAGTYEAESAALSGGAKVNTDHTG
YTGTGFVDGYWTQGATTTFTANVSAAGNYDVTLKYANASGSAKTLSVYVNG
TKIRQTTLASLANWDTWGTKVETLSLNAGNNTIAYKYEASDSGNVNIDSIA
VAPSTSTPVDPEPPITPPTGSNIAIGKAISASSNTQAFVAANANDNDTNTY
WEGGAASSTLTLDLGANQNVTSIVLKLNPSSAWSTRTQTIQVLGHNQSTTT
FSNLVSSQSYTFNPATGNSVTIPVTATVKRLQLSITANSGSGAGQIAEFQV
YGTPAPNPDLTITGMSWTPASPIETDAVTLNATVKNSGNADAPATTVNFYL
NNELVGSSPVGALAAGASSTVSLNVGTKTAATYAVSAKVDESNSIIEQNDA
NNSYTNASSLVVAPVASSDLVGATTWTPSTPVAGNAIGFMVNLKNQGTIAS
ASGAHGITVVVKNAAGAALQSFSGTYSGAIAAGASVNVTLPGTWTAVNGSY
TVTTTVAVDANELTNKQGNNVSTSNLVVYAQRGASMPYSRYDTEDATRGGG
ATLQTAPTFNQAQIASEASGQSYIALPSNGSSAQWTVRQGQGGAGVTMRFT
MPDSTDGMGLNGSLDVYVNGVKVKTVSLTSYYSWQYFSGDMPGDAPSAGRP
LFRFDEVHWKLDTPLQPGDTIKIQKGNGDSLEYGIDFLEIEPVPTAIAKPA
NSLSVTEYGAVANDGQDDLAAFKATVTAAVAAGKSVYIPAGTFNLSSMWEI
GSANNMINNITITGAGYWHTNIQFTNPNAAGGGISLRISGQLDFSNVYMNS
NLRSRYGQNAIYKGFMDNFGTNSKIHDVWVEHFECGMWVGDYAHTPAIYAT
GLVVENSRIRNNLADGINYSQGTSNSIVRNSSIRNNGDDGLAVWTSNTNGA
PAGVNNTFSYNTIENNWRAGGIAFFGGGGHKADHNLIVDTVGGSGIRMNTV
FPGYHFQNNTGITFSDNTLINTGTSQDLYNGERGAIDLEASNDAIKNVTFT
NIDIINTQRDAIQFGYGGGFENIVFNNININGTGLDGVTTSRFAGPHKGAA
IYTYTGNGSATFNNLTTSNVAYPGLNFIQQGFNLVIQ

3. Sequence of mRNA from Paenibacillus curdlanolyticus MP-1

| (SEQ ID NO: 15) | |
|---|---|
| 1 | atgcgcaaca agtatgtcac atggacgctc gccctgacga tgctattttc gagcttcttc |
| 61 | cttgcagtag gtcccaacaa ggtcgttcac gcagcaggcg gaacgaattt agcgctcggc |
| 121 | aaaaacgtta cggcaagcgg ccaatcgcaa acgtatagtc ccaacaatgt aaaagacagc |
| 181 | aatcaatcga cgtactggga aagcacgaac aatgcattcc cgcaatggat tcaagtagac |
| 241 | ttaggcgcaa cgacgagcat tgaccaaatc gtactgaagc tgcccgctgg atggggtacg |
| 301 | cgtacgcaaa cgttagctgt tcaaggaagc acggacggtt cctcgttcac gaatatcgtg |
| 361 | ggctccgcag gctatgtatt taatcctgct gttgccaata acgccgttac gattaacttc |
| 421 | tctgctgcaa gcacgcgtta tgttcgtctg aacgtaacag cgaacacggc ttggccagca |
| 481 | gcgcagctgt ccgaattcga gatttatggc gctggcggca cgacgacgcc tccaacaacg |
| 541 | ccagcaggca catatgaagc tgaatccgca gcattgtccg gcggtgcgaa agtgaacacg |
| 601 | gatcataccg gctacacggg tacgggcttt gttgacggct actggacaca aggcgcgaca |
| 661 | acgacgttca cggctaacgt gtccgcagct ggcaactatg acgttacatt gaaatatgcc |
| 721 | aacgcaagcg gcagtgccaa gacgctaagc gtttacgtca acggcacgaa gattcgccag |
| 781 | acgacgctgg caagcctggc aaactgggac acttggggca cgaaggttga gacgctgagc |
| 841 | ttgaatgccg gcaataatac gattgcatac aagtatgagg ctagcgactc gggcaacgtg |
| 901 | aatatcgact ccattgccgt ggcgccatcg acttcgacac cggtagatcc agaaccgccg |
| 961 | atcacgccgc caacgggcag caatatcgca atcggcaaag cgatcagcgc atcttcgaat |
| 1021 | acgcaagcat tcgtagctgc caacgcgaac gataacgata cgaacacgta ctgggaaggc |
| 1081 | ggagctgcat cgagcacgct gacgctggat cttggcgcga accaaaatgt aacctcgatc |
| 1141 | gtgctgaagc tgaatccttc ttcggcatgg agcacgcgta cgcaaacgat ccaagtgctt |
| 1201 | ggccacaacc aaagcacgac gacgttcagc aatctggtat cttcgcaatc gtatacgttc |
| 1261 | aatcctgcaa cgggcaactc cgtgacgatt ccggttacgg caacagttaa gcgcttgcag |
| 1321 | ctgagcatta cggcgaactc gggttccggc gctggtcaaa ttgcggaatt ccaagtgtat |
| 1381 | ggaacgccgg caccaaaccc agacctgacg atcacaggca tgtcctggac gcctgcttcg |
| 1441 | ccaattgaaa cggatgcagt tacgctgaat gcaacggtta aaaacagcgg aaatgcagac |
| 1501 | gctcctgcaa cgacggtaaa cttctacctg aacaatgagc tcgtaggctc ctcgccagtt |
| 1561 | ggcgcacttg ctgcaggcgc ttcctcgacg gtttcgctga atgttggtac gaaaacggct |
| 1621 | gcaacttatg cagttagcgc gaaagtcgat gagagcaatt cgattatcga gcaaaatgat |
| 1681 | gcgaacaaca gttatacgaa cgcatcctcg ctcgtcgtcg ctcctgtcgc aagctctgac |
| 1741 | ttggttggcg cgacgacgtg gacgcctagc acgccggttg ccggcaatgc aattggcttc |
| 1801 | atggtaaatc ttaaaaacca aggaacgatt gcatctgcaa gcggcgcgca tggcattaca |
| 1861 | gttgtcgtga aaaatgccgc aggcgctgcg ctccaatcgt tcagcggcac ctacagcgga |
| 1921 | gcaatcgcag ctggcgcatc cgttaacgta accctgccag gtacgtggac ggctgtgaat |
| 1981 | ggcagctaca cggtaacgac aacggttgct gtcgatgcta acgagctgac gaacaaacaa |
| 2041 | gggaacaacg taagcacttc gaacctcgtt gtttatgcac aacgtggcgc aagcatgcct |
| 2101 | tacagccgtt atgacacgga agacgctaca cgtggcggcg gtgcaacgct gcaaaccgca |
| 2161 | ccaaccttca accaagcgca aatcgcttcg gaagcatccg gacaaagcta tatcgcgctg |
| 2221 | ccttcgaacg gctcctccgc acaatggacg gtccgtcaag gacaaggcgg agctggcgtt |
| 2281 | acgatgcgct tcacgatgcc ggattcgact gacggtatgg gtttgaacgg ttcgctcgac |
| 2341 | gtttatgtca acggcgttaa agtaaaaacg gtatcgctca cgtcctacta cagctggcag |
| 2401 | tatttctcgg gcgatatgcc tggcgatgcg ccgtccgctg gccgtccgtt gttccgcttt |
| 2461 | gacgaagtac actggaagct tgacacgcct cttcaaccag gcgacacgat caaaatccaa |
| 2521 | aaaggcaacg gagatagcct ggaatacggc attgacttcc tcgaaatcga gccggttcca |
| 2581 | acagcaatcg ctaaacctgc caactcgctt tccgttacgg agtatggcgc tgtagcaaac |
| 2641 | gatggccaag acgaccttgc cgcattcaaa gcaacggtta cggctgcagt tgctgctggc |
| 2701 | aaatccgttt acattcctgc tggcacgttc aatctgagca gcatgtggga aatcggatcg |
| 2761 | gctaacaaca tgatcaacaa cattacgatt acaggcgcag gctactggca tacgaacatt |
| 2821 | caattcacga atccgaatgc agcaggcggc ggcatttcgc tccggatttc cggacagctt |
| 2881 | gatttcagca atgtttacat gaactccaac ctgcgttcgc gttatggtca aaatgcgatt |
| 2941 | tacaaaggct tcatggacaa cttcggcaca aactccaaaa tccatgacgt atgggttgag |
| 3001 | cacttcgagt gcggcatgtg ggtaggcgat tacgcgcata cgccagcgat ctatgcaacg |
| 3061 | ggtcttgtcg ttgaaaacag ccggattcgc aacaaccttg cagacggcat caactactcg |
| 3121 | caaggcacga gcaattcgat cgtacgcaac agcagtatcc gcaataacgg tgatgacggt |
| 3181 | ctggcggttt ggacgagtaa cacgaatggc gcgccagcag gcgtgaacaa cacgttctcg |
| 3241 | tacaacacga tcgaaaacaa ctggcgtgca ggcggtatcg cattcttcgg cggcggcggc |
| 3301 | cacaaggctg accacaacct gatcgttgat acggttggcg gctccggcat ccggatgaac |
| 3361 | acggtattcc caggctacca cttccaaaac aacacgggta ttacgttctc cgacaacacg |
| 3421 | ctgatcaaca caggcacaag ccaagatttg tacaacggcg agcgcggtgc gatcgatctc |
| 3481 | gaagcatcga acgatgcaat caagaacgtc acgttcacga acatcgacat catcaacacc |
| 3541 | cagcgcgatg cgatacaatt cggctacggc ggcggattcg agaacatcgt atttaacaac |
| 3601 | attaacatta acggtacggg gcttgacggc gttacaacct cacggtttgc tggaccgcat |
| 3661 | aaaggtgctg caatctacac gtacacgggc aatggctctg caacgttcaa taacctgacg |
| 3721 | acgagcaacg tggcatatcc aggcttgaat ttcattcagc aaggctttaa tctggtgatc |
| 3781 | cagtag |

III) Paenibacillus sp. Strain RM1.

### 1. General Information of Mutanase

### Genbank E16590; Length: 1,291; Mass (Da): 135 kD

Reference: Shimotsuura I, et al. Appl Environ Microbiol. 2008 May; 74(9):2759-65.2.
The protein sequence of mutanase from Paenibacillus sp. strain RM1

| | [symbol] |
|---|---|
| 1 | [symbol] [symbol] [symbol] [symbol] AAGGPNLTPG KPITASGQSQ |
| 51 | TYSPQNVKDG NQNTYWESTN NAFFQWIQVD LGASTGIDQI VLKLPASWEA |
| 101 | RTQTLAVQGS LNGSTFTDIV GSANYVFSPS VGNNTVTING TATSTPYVRL |
| 151 | YVTANTGWPA AQLSSFEIYG SGDQTPAPDT YQAESAALSG GAKVNTDHAG |
| 201 | YIGTGFVDGY WTQGATTTFS VNAPTAGNYD VTLRYGNATG SNKTVSLYVN |
| 251 | GAKTRQTTLP SLPNWDSWSS KTETLNLNAG SNTIAYKYDP GDSGNVWLDQ |
| 301 | 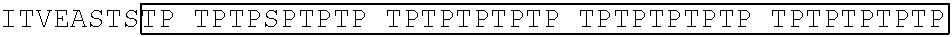 |
| 351 | 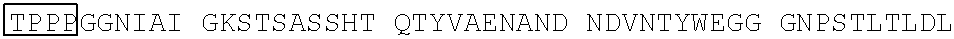 |
| 401 | GANYNITSIV LKLNPSSIWA ARTQTIQVLG HDQNTTTFSN LVSAKSYSFD |
| 451 | PASGNTVTIP VTATVKRLQL NITSNSGAPA GQVAEFQVFG TPAPNPDLTI |
| 501 | TGMSWSPSSP VETDAITLNA TVKNNGNASA AATTVNFYLN NELAGSAPVA |
| 551 | ALAAGASATV PLNVGAKTAA TYAVGAKVDE SNAVTELNES NNSYTNPASL |
| 601 | VVAPVSSSDL VGTVSWTPST PIANNAVSFN VNLKNQGTIA SAGGSHGVTV |
| 651 | VLKNASGSTV QTFSGSYTGS LAPGASVNIT LPGTWTAAAG SYTVTATVAA |
| 701 | DANELPIKQA NNANTASLTV YSARGASMPY SRYDTEDATL GGGATLKSAP |
| 751 | TFDQALTASE ATGQLYAALP SNGSYLQWTV RQGQGGAGVT MRFTMPDSAD |
| 801 | GMGLNGSLDV YVNGTKVKTV SLTSYYSWQY FSGDMPGDAP SAGRPLFRFD |
| 851 | EVHWKLDTPL KPGDTIRIQK NNGDSLEYGV DFIEIEPVPA AISRPANSVS |
| 901 | VTDYGAVPND GQDDLTAFKA AVNAAVASDK ILYIPEGTFH LGNMWEIGSV |
| 951 | SNMIDHITIT CACTWYTNIQ FTNANPASGG ISLPITGKLD FSNVYLNSNL |
| 1001 | RSRYGQNAVY KGFMDNFGTN SVIRDVWVEH FECGFWVGDY GHTPAIRASG |
| 1051 | LLIENSRIRN NLADGVNFAQ GTSNSTVRNS SLRNNGDDAL AVWTSNTNGA |
| 1101 | PEGVNNTFSY NTIENNWRAG GIAFFGGSGH KADHNYIVDC VGGSGIRMNT |
| 1151 | VPPGYHFQNN TGIVFSDTTI VNCGTSRDLY NGEFGAIDLE AGNDAIRNVT |
| 1201 | FTNIDIINSQ RDAIQFGYGG GFTNIVFNNI NINGTGLDGV TTSRFSGPHL |
| 1251 | GAAIFTYTGN GSATFNNLRT SNIAYPNLYY IQSGFNLIIN N |

Deduced amino acid sequence of mutanase RM1. The signal peptide region is underlined, and the linker region is boxed. The arrow indicates the cleavage site for the N-terminal domain of the protein. The DNA sequence was registered as GenBank accession number E16590. (SEQ ID NO: 16)

3. Sequence of mRNA from Paenibacillus sp. Strain RM1

| (SEQ ID NO: 17) | |
|---|---|
| 1 | cccgggtacc agacctatcg ggaaaaacgc gagcggccct tcgcgcctta tgcgctacgg |
| 61 | acggtgctgg cgggcggttt gtttttcatc atcattcccc tgatgatcta cacggcatcg |
| 121 | tatatcccgt ttttgctcgt gccgggtccc ggacacgggt tgaaagacgt cgtctccgcc |
| 181 | cagaagttca tgttcaatta tcatagccgg cttaacgcca cccacccatt ctcgtcgctg |
| 241 | tggtgggagt ggcctctcat ccgcaagccg atctggtatt acggagccgc ggaattggcg |
| 301 | ccgggaaaaa tggcgagcat cgtgggcatg ggcaatccgg cggtgtggtg gacgggaacg |
| 361 | attgcggtaa tcgcggccct tcgctcggcc tggaagaagc gggaccggag catgaccgtc |
| 421 | gtcttcgttg gaatcgcctc gtcttatctt ccgtgggttt tcgtatccag actcaccttt |
| 481 | atttatcact ttttcgcttg cgttccgttt ctcgttcttt gcatcgttta ttggattcga |
| 541 | aaaatggaat agcgtaagcc gggatatcgg attgcgacgc tcctttacgc aggcgcggtt |
| 601 | ctggtgctgt tcattttgtt ttacccgatt ttgtcgggga ccgaaataga cgtttcttac |
| 661 | gcggaccgcg ttctgaagtg gttcggcggg tggatttttc acgggtaagc gagcgttgga |
| 721 | agcaaggaag ggaaggaaga cgagcgtctc cttcccgaaa tccatccaat atcttgaaat |
| 781 | tgcatacatt tttcgtaaga ttgcttctta tctgtctccc tcccctgttc ttataatggg |
| 841 | ggtatcccaa cgaaaggagg gtttgtaagc gctgtcagcs tgtttgccga aagttctcgc |
| 901 | atttgctgac ctacactttg aggaggagga atttaatgcg ctgcaaattt gtcgcatggt |
| 961 | cgcttgttac agccatgctg atggccagtt tgctgacggc tgtaggaccg ttcggccccg |
| 1021 | cttccgccgc gggaggaccg aatctgacgc cgggcaaacc cattacggcg agcggccaat |
| 1081 | cccaaaccta cagccctcag aacgtaaaag acggcaatca aaatacgtat tgggaaagca |
| 1141 | cgaacaacgc gttcccgcaa tggattcaag tggatttggg cgcaagcacg ggcatcgacc |
| 1201 | aaattgtgct gaagctgccc gcaagctggg aagcgcgcac gcaaacgctg gccgttcaag |
| 1261 | gcagcttgaa cggttcgacg ttcacggaca ttgtcggctc cgccaattat gtattcagtc |
| 1321 | cgtctgtcgg gaacaacacg gttacgatca actttaccgc gaccagcacg cgctacgtgc |
| 1381 | gcttgtatgt aacggccaac acgggctggc cggcggcgca gctgtccgaa ttcgaaattt |
| 1441 | acggctccgg cgaccagacg ccggcgcctg atacgtatca agccgaatcc gcggctctgt |
| 1501 | ccggcggcgc gaaagtcaac acggaccatg ccggatatat cggcacgggc tttgttgacg |
| 1561 | gttactggac gcaaggcgcg acgacgacct tttcggtcaa cgcgccgacg gcgggcaact |
| 1621 | acgatgtaac gctgaggtac ggcaacgcaa ccggcagcaa caaaacggta agcctctacg |
| 1681 | tcaatggagc gaagattcgc cagaccacgc tgcccagcct gcctaactgg gattcatgga |
| 1741 | gcagcaagac ggagacgctt aacctgaatg caggcagcaa caccattgcg tacaaatacg |
| 1801 | acccgggcga ttccggcaac gtcaatcttg accaaatcac ggtcgaagcg tcgacttcaa |
| 1861 | cgcctactcc tactccatcc cctactccta cacctacgcc aacgccgacg cctacgccta |
| 1921 | cgcctacacc cacacctact ccgaccccga cgcctacgcc tacacctaca cctacaccta |
| 1981 | cgccgacgcc tcctccgggc ggcaacatcg ccatcggcaa atcgatttcc gcatcctccc |
| 2041 | acacgcagac gtacgttgcg gagaacgcga acgataacga tgtcaacacg tactgggaag |
| 2101 | gcggcggcaa tccgagcacg ctgacgctcg atctcggagc gaactacaat attacgtcca |
| 2161 | tcgtgctgaa gctgaacccg tcctcgatat gggctgcgcg tacgcaaacg attcaagtgc |
| 2221 | tcggacacga tcagaacacg acgaccttca gcaatctggt ctcggcgaaa tcgtactcgt |
| 2281 | tcgatccggc ctccggcaat actgtgacca ttccggttac ggcgacggtg aaacgtttgc |
| 2341 | agttgaacat tacgtcgaac tccggcgccc cggccggaca agtcgccgag ttccaggtgt |
| 2401 | tcggcacgcc tgcgccgaat ccggacctga cgattaccgg catgtcctgg tcgccttctt |
| 2461 | ctccggttga gaccgacgcc attacgctaa acgcaacggt gaagaacaac gggaatgcca |
| 2521 | gcgccgcggc gaccaccgtc aatttctacc tgaacaacga gctggcgggt tccgcgccgg |
| 2581 | tagccgcgct ggcggcaggc gcttcggcaa cggtgccgct gaatgtcggc gcgaaaaccg |
| 2641 | ccgcgacata cgcggtcggc gccaaagtag acgagagcaa cgcggtcatc gagctgaacg |
| 2701 | agtcgaacaa cagctacacg aatccggctt cactcgttgt ggcccccgtt tccagctcgg |
| 2761 | atctggtggg cacggtttcg tggacgccga gcactccgat tgccaacaat gccgtttctt |
| 2821 | ttaacgtaaa tcttaaaaat caaggaacga ttgcttccgc cggcgggtct cacggcgtga |
| 2881 | cggtcgtgct taaaaatgct tccggttcga ccgttcaaac gttcagcggt tcctataccg |
| 2941 | gcagcctggc tccgggagcg tccgtcaaca tcacccttcc ggggacctgg acggcggcag |
| 3001 | ccggcagcta cacggtaacg gccaccgttg cggcagacgc caacgaactt ccgatcaagc |
| 3061 | aagccaacaa cgcgaacacc gcaagcctga ccgtatattc cgcccgcggc gcgagcatgc |
| 3121 | cgtacagccg gtatgacacc gaggacgcca ccctcggcgg cggcgccacg ctgaagtccg |
| 3181 | cgccgacatt cgatcaggcg cttacggcat cggaagccac cggccaactc tatgcggcgc |
| 3241 | tgccctcgaa cggctcctat cttcaatgga ccgtcagaca gggtcagggc ggcgcaggcg |
| 3301 | tgacgatgag atttacgatg cccgactcgg cggacggcat gggattaaac ggttcgctag |
| 3361 | acgtttacgt caacggcacc aaagtcaaaa ccgtatcgct gacctcctac tacagctggc |
| 3421 | agtatttctc gggcgatatg cccggagacg ctcccagcgc gggccgtccg ctcttccgct |
| 3481 | ttgacgaagt gcactggaag ctggatactc cgctcaaacc cggagacacg attcgcatcc |
| 3541 | agaagaacaa cggcgacagc ctggaatacg gtgtcgactt tattgaaatc gaaccggttc |
| 3601 | cggctgcgat ctcccgtccg gccaactcgg tttccgtaac ggattacggc gctgtgccga |
| 3661 | acgacggaca ggacgatctc accgccttta aagccgccgt aaacgcggcg gtcgcatccg |
| 3721 | acaagatctt gtacattccg gaaggaacgt tccacctcgg caacatgtgg gagatcggtt |
| 3781 | ccgtcagcaa catgatcgat cacattacga ttacgggagc cggtatctgg tatacgaaca |
| 3841 | tccagtttac caacgccaat ccggcgtccg gcggcatctc gctccggatt acgggcaagc |
| 3901 | ttgatttcag caacgtgtac ctcaactcca atttgcggtc gcggtatggt caaaatgcgg |
| 3961 | tttacaaagg ctttatggac aacttcggga ccaattccgt catccgcgac gtctgggtcg |
| 4021 | agcacttcga atgcggcttc tgggtcgggg actacgggca tacgccggcg atccgcgcga |
| 4081 | gcgggctgct gattgaaaac agccgaatcc gcaacaacct ggccgatggc gtcaacttcg |
| 4141 | cccaagggac cagcaattcg accgtacgca acagcagcct gcgcaacaac ggcgacgacg |
| 4201 | cccttgccgt atggacgagt aatacgaacg gcgcgcccga aggcgtaaac aataccttct |
| 4261 | cgtacaacac catcgaaaac aactggcgcg cgggaggcat cgccttcttc ggaggaagcg |
| 4321 | gacacaaggc cgaccacaac tacatcgtcg actgcgtcgg cggttccggc atccggatga |
| 4381 | acaccgtgtt ccccggatac cacttccaga acaataccgg cattgtgttc tcggacacga |
| 4441 | ccatcgtcaa ctgcggcacg agcaaagacc tatacaacgg cgaacgcggc gccatcgatc |
| 4501 | tggaagcttc gaacgacgcc atccggaacg tgacgtttac caacatcgat attatcaact |
| 4561 | ctcagcgcga tgcgatccag ttcggttacg gcggcggctt caccaacatc gtgttcaaca |
| 4621 | acatcaacat taacggaacc ggtcttgacg gcgtaaccac ctcgcggttc tcgggaccgc |
| 4681 | atctgggcgc ggcgatcttc acctataccg gcaacggctc cgccacgttc aacaatctga |
| 4741 | ggaccagcaa tatcgcttac cccaatctgt attacatcca gagcgggttc aatctgatca |
| 4801 | tcaataatta gatatctggg cccgtctgcg ggggaggaac tcttcggagc tcgaattcgt |
| 4861 | aatcatggtc atagctgttt cctgtgtgaa attgttatcc gctcacaatt ccacacaaca |
| 4921 | tacgagccgg aagcataaag tgtaaagcct ggggtgccta atgagtgagc taactcacat |
| 4981 | taattgcgtt gcgctcactg cccgctttcc agtcgggaaa ctgtcgtgcc agctgcatta |
| 5041 | atgaatcggc caacgcgcgg ggagaggcsg tttkcgtatt gggcgccctt |

IV) Trichoderma harzianum (CCM F-470)
1. General Information of Mutanase from Trichoderma harzianum
Also see the world wide web at uniprot.org/uniprot/Q8WZM7Length: 635

### Mass (Da): 67,726

Last modified: Mar. 1, 2002-v1
Checksum: iBB0D864E2F432C58
2. The Protein Sequence of Mutanase from Trichoderma harzianum
See the world wide web at uniprot.org/uniprot/Q8WZM7.fasta

>tr|Q8WZM7|Q8WZM7_TRIHA Alpha-1,3-glucanase
OS = Trichoderma harzianum GN = p3 PE = 2 SV = 1
(SEQ ID NO: 18)
MLGVFRRLRLGALAAAALSSLGSAAPANVAIRSLEERASSADRLVFCHFMI
GIVGDRGSSADYDDDMQRAKAAGIDAFALNIGVDGYTDQQLGYAYDSADRN
GMKVFISFDFNWWSPGNAVGVGQKIAQYANRPAQLYVDNRPFASSFAGDGL
DVNALRSAAGSNVYFVPNFHPGQSSPSNIDGALNWMAWDNDGNNKAPKPGQ
TVTVADGDNAYKNWLGGKPYLAPVSTWVFNHFGPEVSYSKNWVFPSGPLIY
NRWQQVLQQGFPRVEIVTWNDYGESHYVGPLKSKQFHDGNSKWVNDMPHDG
FLDLSKPFIAAYKNRDTDISKYVQNEQLVYWYRRNLKALDCDATDTTSNRP
ANNGSGNYFEGRPDGWQTMDDTVYVAALLKTAGSVTVTSGGTTQTFQANAG
ANLFQIPASIGQQKFALTRNGQTVFSGTSLMDITNVCSCGIYNFNPYVGTI
PAGFDDPLQADGLFSLTIGLHVTTCQAKPSLGTNPPVTSGPVSSLPASSTT
RASSPPPVSSTRVSSPPVSSPPVSRTSSAPPPPGNSTPPSGQVCVAGTVAD
GESGNYIGLCQFSCNYGYCPPGPCKCTAFGAPISPPASNGRNGCPLPGEGD
GYLGLCSFSCNHNYCPPTACQYC

3. Sequence of mRNA (Trichoderma harzianum
See the world wide web at ebi.ac.uk/ena/data/view/AJ243799&display=fasta)

>ENA|AJ243799|AJ243799.1 Trichoderma harzianum
mRNA for alpha-1,3-glucanase (p3 gene)
(SEQ ID NO: 19)
ATGTTGGGCGTTTTCCGCCGCCTCAGGCTCGGCGCCCTTGCCGCCGCAGCT
CTGTCTTCTCTCGGCAGTGCCGCTCCCGCCAATGTTGCTATTCGGTCTCTC
GAGGAACGTGCTTCTTCTGCTGACCGTCTCGTATTCTGTCATTTCATGATT
GGGATCGTGGGTGACCGTGGCAGCTCGGCAGATTATGATGACGATATGCAA
CGTGCCAAAGCCGCTGGCATTGACGCCTTCGCCCTGAACATCGGCGTTGAC
GGCTATACCGACCAGCAGCTCGGCTATGCCTATGACTCTGCCGATCGTAAT
GGCATGAAAGTCTTCATTTCATTTGATTTCAACTGGTGGAGCCCCGGCAAT
GCAGTTGGTGTTGGCCAGAAGATTGCGCAGTATGCCAACCGCCCTGCCCAG
CTGTATGTCGACAACCGGCCATTCGCCTCTTCCTTCGCCGGTGACGGTCTG
GATGTAAATGCGTTGCGCTCTGCTGCAGGCTCCAACGTTTACTTTGTGCCC
AACTTCCACCCTGGTCAATCTTCCCCCTCCAACATTGATGGCGCCCTTAAC
TGGATGGCCTGGGATAATGATGGAAACAACAAGGCACCCAAGCCGGGCCAG
ACTGTCACAGTGGCAGACGGTGACAACGCTTATAAGAATTGGTTGGGTGGC
AAGCCTTACCTGGCGCCTGTCTCAACTTGGGTTTTCAACCATTTCGGGCCC
GAAGTTTCATATTCCAAGAACTGGGTTTTCCCAAGTGGGCCTCTGATCTAT
AACCGGTGGCAACAAGTCTTGCAGCAAGGGTTCCCAAGGGTTGAGATCGTT
ACCTGGAATGACTACGGGGAATCTCACTACGTCGGTCCCCTGAAGTCTAAG
CAATTTCATGATGGGAACTCCAAATGGGTCAATGATATGCCCCACGATGGA
TTCCTGGATCTTTCGAAGCCGTTCATAGCCGCATATAAAAACAGGGATACC
GACATCTCCAAGTATGTTCAAAATGAGCAGCTTGTTTACTGGTACCGCCGC
AACTTAAAGGCACTGGACTGTGACGCCACCGACACAACCTCTAACCGCCCG
GCTAACAATGGAAGCGGCAATTACTTTGAGGGACGCCCCGATGGTTGGCAA
ACTATGGATGATACGGTTTACGTGGCGGCACTTCTCAAGACTGCCGGTAGC
GTCACGGTCACGTCTGGTGGCACCACTCAAACGTTCCAGGCCAACGCCGGA
GCCAATCTCTTCCAAATCCCGGCCAGCATCGGCCAGCAAAAGTTTGCTCTG
ACTCGTAACGGTCAGACCGTCTTTAGCGGAACCTCATTGATGGATATCACC
AACGTTTGCTCTTGCGGTATCTACAACTTCAACCCATATGTTGGCACCATT
CCTGCCGGCTTTGACGACCCTCTTCAGGCTGACGGTCTTTTCTCTTTGACC
ATCGGATTGCACGTCACAACTTGTCAGGCCAAGCCATCTCTTGGAACTAAC
CCTCCTGTCACTTCCGGCCCTGTGTCCTCGCTTCCAGCTTCCTCCACCACC
CGCGCATCCTCGCCGCCTCCTGTTTCTTCAACTCGTGTCTCTTCTCCCCCT
GTCTCTTCCCCTCCAGTTTCTCGCACCTCTTCTGCCCCTCCCCCTCCGGGC
AACAGCACGCCGCCATCGGGTCAGGTTTGCGTTGCCGGCACCGTTGCCGAC
GGCGAGTCTGGCAACTACATCGGCCTGTGCCAATTCAGCTGCAACTACGGT
TACTGCCCACCAGGACCGTGTAAGTGCACCGCCTTTGGTGCTCCCATCTCG
CCACCGGCATCCAACGGCCGCAACGGCTGCCCTCTGCCGGGAGAAGGCGAT
GGTTATCTGGGCCTGTGCAGTTTCAGTTGTAACCATAATTACTGCCCGCCA
ACGGCATGTCAATACTGCTAGGAGGGATCAATCTCAGTATGAGTATATGGA
GGCTGCTGAAGGACCAATTAGCTGTTCTTATCGGCAGACGAAACCCATAGA
GTAAGAAGTTAAATAAAATGCAATTAATGTGTTTTCAAAAAAAAAAAAAAA
A

(There is a polyA tail since Trichoderma harzianum is fungi)
V) Trichoderma harzianum
1. General Information of Mutanase from Trichoderma harzianum
Also see the world wide web at uniprot.org/uniprot/Q8WZM7;
2. The Protein Sequence of Mutanase from Trichoderma harzianum
See: the world wide web at uniprot.org/uniprot/Q8MZM7.fasta)

>tr|Q8WZM7|Q8WZM7_TRIHA Alpha-1,3-glucanase
OS = Trichoderma harzianum GN = p3 PE = 2 SV = 1
(SEQ ID NO: 20)
MLGVFRRLRLGALAAAALSSLGSAAPANVAIRSLEERASSADRLVFCHFMI
GIVGDRGSSADYDDDMQRAKAAGIDAFALNIGVDGYTDQQLGYAYDSADRN
GMKVFISFDFNWWSPGNAVGVGQKIAQYANRPAQLYVDNRPFASSFAGDGL
DVNALRSAAGSNVYFVPNFHPGQSSPSNIDGALNWMAWDNDGNNKAPKPGQ
TVTVADGDNAYKNWLGGKPYLAPVSTWVFNHFGPEVSYSKNWVFPSGPLIY
NRWQQVLQQGFPRVEIVTWNDYGESHYVGPLKSKQFHDGNSKWVNDMPHDG
FLDLSKPFIAAYKNRDTDISKYVQNEQLVYWYRRNLKALDCDATDTTSNRP
ANNGSGNYFEGRPDGWQTMDDTVYVAALLKTAGSVTVTSGGTTQTFQANAG
ANLFQIPASIGQQKFALTRNGQTVFSGTSLMDITNVCSCGIYNFNPYVGTI
PAGFDDPLQADGLFSLTIGLHVTTCQAKPSLGTNPPVTSGPVSSLPASSTT
RASSPPPVSSTRVSSPPVSSPPVSRTSSAPPPPGNSTPPSGQVCVAGTVAD
GESGNYIGLCQFSCNYGYCPPGPCKCTAFGAPISPPASNGRNGCPLPGEGD
GYLGLCSFSCNHNYCPPTACQYC

3. Sequence of mRNA (Trichoderma harzianum Further Information can be Found at the World Wide Web at ebi.ac.uk/ena/data/view/AJ243799&display=fasta)

>ENA|AJ243799|AJ243799.1 Trichoderma harzianum
mRNA for alpha-1,3-glucanase (p3 gene)
(SEQ ID NO: 21)
ATGTTGGGCGTTTTCCGCCGCCTCAGGCTCGGCGCCCTTGCCGCCGCAGCT
CTGTCTTCTCTCGGCAGTGCCGCTCCCGCCAATGTTGCTATTCGGTCTCTC
GAGGAACGTGCTTCTTCTGCTGACCGTCTCGTATTCTGTCATTTCATGATT
GGGATCGTGGGTGACCGTGGCAGCTCGGCAGATTATGATGACGATATGCAA
CGTGCCAAAGCCGCTGGCATTGACGCCTTCGCCCTGAACATCGGCGTTGAC
GGCTATACCGACCAGCAGCTCGGCTATGCCTATGACTCTGCCGATCGTAAT
GGCATGAAAGTCTTCATTTCATTTGATTTCAACTGGTGGAGCCCCGGCAAT
GCAGTTGGTGTTGGCCAGAAGATTGCGCAGTATGCCAACCGCCCTGCCCAG
CTGTATGTCGACAACCGGCCATTCGCCTCTTCCTTCGCCGGTGACGGTCTG
GATGTAAATGCGTTGCGCTCTGCTGCAGGCTCCAACGTTTACTTTGTGCCC
AACTTCCACCCTGGTCAATCTTCCCCCTCCAACATTGATGGCGCCCTTAAC
TGGATGGCCTGGGATAATGATGGAAACAACAAGGCACCCAAGCCGGGCCAG
ACTGTCACAGTGGCAGACGGTGACAACGCTTATAAGAATTGGTTGGGTGGC
AAGCCTTACCTGGCGCCTGTCTCAACTTGGGTTTTCAACCATTTCGGGCCC
GAAGTTTCATATTCCAAGAACTGGGTTTTCCCAAGTGGGCCTCTGATCTAT
AACCGGTGGCAACAAGTCTTGCAGCAAGGGTTCCCAAGGGTTGAGATCGTT
ACCTGGAATGACTACGGGGAATCTCACTACGTCGGTCCCCTGAAGTCTAAG
CAATTTCATGATGGGAACTCCAAATGGGTCAATGATATGCCCCACGATGGA
TTCCTGGATCTTTCGAAGCCGTTCATAGCCGCATATAAAAACAGGGATACC
GACATCTCCAAGTATGTTCAAAATGAGCAGCTTGTTTACTGGTACCGCCGC
AACTTAAAGGCACTGGACTGTGACGCCACCGACACAACCTCTAACCGCCCG
GCTAACAATGGAAGCGGCAATTACTTTGAGGGACGCCCCGATGGTTGGCAA
ACTATGGATGATACGGTTTACGTGGCGGCACTTCTCAAGACTGCCGGTAGC
GTCACGGTCACGTCTGGTGGCACCACTCAAACGTTCCAGGCCAACGCCGGA
GCCAATCTCTTCCAAATCCCGGCCAGCATCGGCCAGCAAAAGTTTGCTCTG
ACTCGTAACGGTCAGACCGTCTTTAGCGGAACCTCATTGATGGATATCACC
AACGTTTGCTCTTGCGGTATCTACAACTTCAACCCATATGTTGGCACCATT
CCTGCCGGCTTTGACGACCCTCTTCAGGCTGACGGTCTTTTCTCTTTGACC
ATCGGATTGCACGTCACAACTTGTCAGGCCAAGCCATCTCTTGGAACTAAC
CCTCCTGTCACTTCCGGCCCTGTGTCCTCGCTTCCAGCTTCCTCCACCACC
CGCGCATCCTCGCCGCCTCCTGTTTCTTCAACTCGTGTCTCTTCTCCCCCT
GTCTCTTCCCCTCCAGTTTCTCGCACCTCTTCTGCCCCTCCCCCTCCGGGC
AACAGCACGCCGCCATCGGGTCAGGTTTGCGTTGCCGGCACCGTTGCCGAC
GGCGAGTCTGGCAACTACATCGGCCTGTGCCAATTCAGCTGCAACTACGGT
TACTGCCCACCAGGACCGTGTAAGTGCACCGCCTTTGGTGCTCCCATCTCG
CCACCGGCATCCAACGGCCGCAACGGCTGCCCTCTGCCGGGAGAAGGCGAT
GGTTATCTGGGCCTGTGCAGTTTCAGTTGTAACCATAATTACTGCCCGCCA
ACGGCATGTCAATACTGCTAGGAGGGATCAATCTCAGTATGAGTATATGGA
GGCTGCTGAAGGACCAATTAGCTGTTCTTATCGGCAGACGAAACCCATAGA
GTAAGAAGTTAAATAAAATGCAATTAATGTGTTTTCAAAAAAAAAAAAAAA
A

(There is a polyA tail since Trichoderma harzianum is fungi)

Dextranase (Dex) Gene from Penicillium minioluteum

### GenBank: L41562.1

(see the world wide web at ncbi.nlm.nih.gov/nuccore/L41562.1)
The mature protein has 574 amino acids with MW at 67KD. The optimum reaction condition is pH 5.5 and 40° C. The pH range is 3-6.

| Amino acid sequence | |
|---|---|
| (SEQ ID NO: 22) | |
| MATMLKLLALTLAISESAIGAVMHPPGNSHPGTHMGTTNNTHCG | |
| ADFCTWWHDSGEINTQTPVQPGNVRQSHKYSVQVSLAGTNNFHDSFVYESIPRNGNGR | |
| IYAPTDPPNSNTLDSSVDDGISIEPSIGLNMAWSQFEYSHDVDVKILATDGSSLGSPS | |
| DVVIRPVSISYAISQSDDGGIVIRVPADANGRKFSVEFKTDLYTFLSDGNEYVTSGGS | |
| VVGVEPTNALVIFASPFLPSGMIPHMTPDNTQTMTPGPINNGDWGAKSILYFPPGVYW | |
| MNQDQSGNSGKLGSNHIRLNSNTYWVYLAPGAYVKGAIEYFTKQNFYATGHGILSGEN | |
| YVYQANAGDNYIAVKSDSTSLRMWWHNNLGGGQTWYCVGPTINAPPFNTMDFNGNSGI | |
| SSQISDYKQVGAFFFQTDGPEIYPNSVVHDVFWHVNDDAIKIYYSGASVSRATIWKCH | |
| NDPIIQMGWTSRDISGVTIDTLNVIHTRYIKSETVVPSAIIGASPFYASGMSPDSRKS | |
| ISMTVSNVVCEGLCPSLFRITPLQNYKNFVVKNVAFPDGLQTNSIGTGESIIPAASGL | |
| TMGLNISNWTVGGQKVTMENFQANSLGQFNIDGSYWGEWQIS | |
| DNA sequence | |
| (SEQ ID NO: 23) | |
| 1 | ggcatagtaa tcccgacagc cgagtatgat ggagcttctt cggataatga tagcgccacc |
| 61 | agaccttgct tgagctggag agctaaaaca ttaaacgcca cacgaccaac actctcatta |
| 121 | gttgcgatag atgatgctcg gagctgttga aactcagaaa ttccttctat gcggggtctc |
| 181 | caagatcgat cctgggggat gtgaatacta cggtggacct aattgacgcc ttgacaggtg |
| 241 | atgttaagcg aaccaaggaa gaataatctg gggctagatg aagatgttga gctgtaaggt |
| 301 | acggtacgtt cctattggct ttatcggagc ttctccgggt tactcagtct ttccgggagc |
| 361 | atgatcattt ttgtattgtc caatagtaag cagaaactga gagccaccac aaactcaaaa |
| 421 | cctcggtagc gaagtttccc ggaaccagtc aggattctca gaaactgtgc tcgtgttgcg |
| 481 | gggaatccgc attctacgtc gtctggagca aggaaatgtt cgtgctggat tgaggaggat |
| 541 | aggtaggttg gagaatctct tcagctaacc aatctataag catgctccgg taacctttag |
| 601 | agtttcacat tcaacgtaat ttccaagata gccagagcgt ccttgaatta ctatgtagaa |
| 661 | atcctaaaat ttcccctgta aaatgcaagt caacgagatg cgtgccctca atgtctctcg |
| 721 | gcgctacccc ggaaatgatg cataaggcca agaatgtcac ccggtaactt tttcttcaga |
| 781 | atatcctaag atttccatca aacacagtcg aataggtcaa tgctcgcgag agactttctg |
| 841 | ccttcactct acgtcctact catagaagtt caacggctca attccggggt aatctagagt |
| 901 | ttggacctca agggagatgt tgcaacaaat tgtactagaa cgatgcgctt gctttccaat |
| 961 | acagtagttg acttcatata gcttccaaca aaagggatgg ggatgaaggc tctatagcga |
| 1021 | gaagtctata agaaagtgtc ctcatacctg tatctctcag tcgttcgaga acaatcccgg |
| 1081 | aaactatctt atcttgcgag aaagaagaca atatctcaaa cttatggcca caatgctaaa |
| 1141 | gctacttgcg ttgacccttg caattagcga gtccgccatt ggagcagtca tgcacccacc |
| 1201 | tggcaattct catcccggta cccatatggg cactacgaat aatacccatt gcggcgccga |
| 1261 | tttctgtacc tggtggcatg attcagggga gatcaatacg cagacacctg tccaaccagg |
| 1321 | gaacgtgcgc caatctcaca agtattccgt gcaagtgagc ctagctggta caaacaattt |
| 1381 | tcatgactcc tttgtatatg aatcgatccc ccggaacgga aatggtcgca tctatgctcc |
| 1441 | caccgatcca cccaacagca acacactaga ttcaagtgtg gatgatggaa tctcgattga |
| 1501 | gcctagtatc ggccttaata tggcatggtc ccaattcgag tacagccacg atgtagatgt |
| 1561 | aaagatcctg gccactgatg gctcatcgtt gggctcgcca agtgatgttg ttattcgccc |
| 1621 | cgtctcaatc tcctatgcga tttctcagtc tgacgatggt gggattgtca tccgggtccc |
| 1681 | agccgatgcg aacggccgca aattttcagt tgagttcaaa actgacctgt acacattcct |
| 1741 | ctctgatggc aacgagtacg tcacatcggg aggcagcgtc gtcggcgttg agcctaccaa |
| 1801 | cgcacttgtg atcttcgcaa gtccgtttct tccttctggc atgattcctc atatgacacc |
| 1861 | cgacaacacg cagaccatga cgccaggtcc tatcaataac ggcgactggg gcgccaagtc |
| 1921 | aattctttac ttcccaccag gtgtatactg gatgaaccaa gatcaatcgg gcaactcggg |
| 1981 | gaagttagga tctaatcata tacgtctaaa ctcgaacact tactgggtct accttgcccc |
| 2041 | cggtgcgtac gtgaagggtg ctatagagta ttttaccaag cagaacttct atgcaactgg |
| 2101 | tcatggtatc ctatcgggtg aaaactatgt ttaccaagcc aatgccggcg acaactacat |
| 2161 | tgcagtcaag agcgattcaa ccagcctccg gatgtggtgg cacaataacc ttgggggtgg |
| 2221 | tcaaacatgg tactgcgttg gcccgacgat caatgcgcca ccattcaata ctatggattt |
| 2281 | caatggaaat tctggcatct caagtcaaat tagcgactat aagcaggtgg gagccttctt |
| 2341 | cttccagacg gatggaccag aaatatatcc caatagtgtc gtgcacgacg tcttctggca |
| 2401 | cgtcaatgat gatgcaatca aaatctacta ttcgggagca tctgtatcgc gggcaacgat |
| 2461 | ctggaaatgt cacaatgacc caatcatcca gatgggatgg acgtctcggg atatcagtgg |
| 2521 | agtgacaatc gacacattaa atgttattca cacccgctac atcaaatcgg agacggtggt |
| 2581 | gccttcggct atcattgggg cctctccatt ctatgcaagt gggatgagtc ctgattcaag |
| 2641 | aaagtccata tccatgacgg tttcaaacgt tgtttgcgag ggtctttgcc cgtccctatt |
| 2701 | ccgcatcaca ccccttcaga actacaaaaa ttttgttgtc aaaaatgtgg ctttcccaga |
| 2761 | cgggctacag acgaatagta ttggcacagg agaaagcatt attccagccg catctggtct |
| 2821 | aacgatggga ctgaatatct ccaactggac tgttggtgga caaaaagtga ctatggagaa |
| 2881 | ctttcaagcc aatagcctgg ggcagttcaa tattgacggc agctattggg gggagtggca |
| 2941 | gattagctga attccagctc tcggagcgcg tgagtgcttc tacccgctcc tttacccttg |
| 3001 | tcgagagata aaggcataag ttagctcatg tgaaggcgat ttcagttcat tctctctttt |
| 3061 | tggagcttat ttcctgttcg accaattgtg acaccaactt gcctttcaaa agacgtggac |
| 3121 | gatatgtgta cggtaatcag tcaaatgaac gtcaacattc atttaataag gacatttcca |
| 3181 | ggtttcctta ctctgtcgat tatgcctaac tcgggttgat gtcttgtcag gatggaaaat |
| 3241 | ctcgttgtgt acttccagtg aaatgggcag ggctaagccc taaaccctaa cgcatacaat |
| 3301 | ttgtaggcac ctacccatgt aagttcacac ccagtcgact tataagtcta gatatttatg |
| 3361 | ctatgcaggc tctggaatga tttacattcc atgctataca tagttatttg caagaatttg |
| 3421 | cagacgagat aaaaatcaat ggacgaataa tcacgcatta ctccacaggc tcatgccacg |
| 3481 | gagcaagggt tcccccgaat ctaggccaga ccgggatgat attcaaccga ttctttttgc |
| 3541 | agtaactatc tccgtacgag ctgcacgagc taaacggatt atataaaggt gctaactgag |
| 3601 | cattggatcc gtcagttata tgaaatgca |

2. Dextranase (Dex) Gene from Penicillium aculeatum (Talaromyees aculeatus Strain z01)
GenBank: KF999646.1 (see the world wide web at ncbi.nlm.nih.gov/nuccore/KF999646.1)
The optimum pH is around 5. The pH range is 3-6.

| Amino acid sequence | |
|---|---|
| (SEQ ID NO: 24) | |
| MATMLKLLTLALAISESAIGAVLHPPGSSHPSTRTDTTNNTHCG | |
| ADFCTWWHDSGEINTQTPVQPGNVRQSHKYSVQVSLAGANNFQDSFVYESIPRNGNGR | |
| IYAPTDPPNSNTLDSSVDDGISIEHSIGLNMAWSQFEYSQDVDIKILAADGSSLGSPS | |
| DVVIRPVSISYAISQSDDGGIVIRVPADANGRKFSVEFKNDPYTFLSDGNEYVTSGGS | |
| VVGVEPTNALVIFASPFLPSGMIPHMTPDNTQTMTPGPINNGDWGSKSILYFPPGVYW | |
| MNQDQSGNSGKLGSNHIRLNSNTYWVYFAPGAYVKGAIEYFTKQNFYATGHGVLSGEN | |
| YVYQANAGENYVAVKSDSTSLRMWWHNNLGGGQTWYCVGPTINAPPFNTMDFNGNSGI | |
| SSQISDYKQVGAFFFQTDGPEIYPNSVVHDVFWHVNDDAIKIYYSGASVSRATIWKCH | |
| NDPIIQMGWTSRDISGVTIDTLNVIHTRYIKSETVVPSAIIGASPFYASGMSPDSSKS | |
| ISMTVSNVVCEGLCPSLFRITPLQNYKNFVVKNVAFPDGLQTNSIGTGESIIPAASGL | |
| TMGLDISNWSVGGQKVTMQNFQANSLGQFDIDGSYWGEWQIN | |
| DNA sequence | |
| (SEQ ID NO: 25) | |
| 1 | atggccacaa tgctaaagct acttacgttg gcccttgcaa ttagcgagtc tgccattgga |
| 61 | gcagtcctgc acccacctgg cagttctcat cccagtaccc gtacggacac tacgaataat |
| 121 | acccattgcg gtgccgactt ctgtacctgg tggcatgatt caggcgagat caacacacag |
| 181 | acacctgtcc aaccggggaa cgtgcgccaa tctcacaagt attccgtaca agtgagccta |
| 241 | gctggtgcga acaactttca ggactccttt gtatatgaat cgatccctcg gaacggaaat |
| 301 | ggtcgcatct atgctcccac cgatccaccc aacagcaaca cactagattc aagtgttgat |
| 361 | gatggaatct cgattgaaca tagtattggc ctcaatatgg catggtccca attcgagtac |
| 421 | agccaggatg tcgatataaa gatcctggcc gctgatggct catcgttggg ctcaccaagt |
| 481 | gatgttgtta ttcgccccgt ctcaatctcc tatgcaattt ctcaatccga cgatggcgga |
| 541 | attgtcattc gggtcccagc cgatgcgaac ggccgcaaat tttcagtcga gttcaaaaat |
| 601 | gacccgtaca cgttcctctc tgacggcaac gagtacgtca catcgggagg cagcgttgtc |
| 661 | ggcgttgagc ctaccaacgc acttgtgatc ttcgcaagcc cgtttcttcc gtcaggcatg |
| 721 | attcctcata tgacacccga caacacgcag accatgacac caggacctat caataacggc |
| 781 | gactggggct ccaagtcaat tctttatttc ccaccgggcg tatactggat gaaccaagat |
| 841 | caatcaggca actcggggaa attaggatct aatcatatac gcctgaactc gaacacctac |
| 901 | tgggtctact ttgccccagg tgcgtacgtg aagggtgcta tagagtattt caccaagcag |
| 961 | aacttctatg caactggtca tggtgtccta tcgggtgaaa actatgttta ccaagccaat |
| 1021 | gctggcgaaa actacgttgc ggtcaagagc gattcgacta gcctccggat gtggtggcac |
| 1081 | aataacctgg gaggtggaca aacatggtac tgcgttgggc ctacgatcaa tgcgccgcca |
| 1141 | tttaacacaa tggatttcaa tggaaattcc ggtatctcaa gtcaaattag cgactataag |
| 1201 | caggtgggag ctttcttctt tcagacggat ggaccagaaa tttatcccaa tagtgtcgtg |
| 1261 | cacgacgtct tctggcatgt caatgatgat gcaatcaaaa tctactattc cggagcatct |
| 1321 | gtctcgcggg caacgatctg gaaatgtcac aacgatccaa tcatccagat gggatggacg |
| 1381 | tctcgggata tcagtggagt gacaatcgac acattgaatg tcatccacac ccgctacatc |
| 1441 | aagtcggaga cggtggtgcc ttcggctatc attggggctt ctccattcta tgcaagtggg |
| 1501 | atgagtcctg attcaagcaa gtctatatcc atgacggttt caaacgttgt ctgcgaggga |
| 1561 | ctttgcccgt ctctgttccg aatcacacct ttacagaact acaagaattt tgttgtcaaa |
| 1621 | aatgtggctt tcccagatgg gctacagacg aatagtattg gcacgggaga aagcattatt |
| 1681 | ccagccgcat ctggtctaac gatgggactg gatatctcca actggtctgt tggtggtcag |
| 1741 | aaggtgacta tgcagaactt tcaagccaat agtctggggc aattcgacat tgacggcagc |
| 1801 | tattgggggg agtggcagat taactagctg aataatattg cagctttcag ggcgcatgag |
| 1861 | tgcttgtacc cgctccttta cccttgtc |

3. Penicillium funiculosum dexA Gene for dextranase
GenBank: AJ272066.1 (see the world wide web at ncbi.nlm.nih.gov/nuccore/7801166)
The optimum pH is around 5.5. The optimum temperature is 60° C. The pH range is 5-7.5 (see the world wide web at sciencedirect.com/science/article/pii/S0032959298001277)

| Amino acid sequence | |
|---|---|
| (SEQ ID NO: 26) | |
| MATMLKLLALTLAISESAIGAVMHPPGVSHPGTHTGTTNNTHCG | |
| ADFCTWWHDSGEINTQTPVQPGNVRQSHKYSVQVSLAGTNNFHDSFVYESIPRNGNGR | |
| IYAPTDPSNSNTLDSSVDDGISIEPSIGLNMAWSQFEYSQDVDIKILATDGSSLGSPS | |
| DVVIRPVSISYAISQSNDGGIVIRVPADANGRKFSVEFKNDLYTFLSDGNEYVTSGGS | |
| VVGVEPTNALVIFASPFLPSGMIPHMKPHNTQTMTPGPINNGDWGAKSILYFPPGVYW | |
| MNQDQSGNSGKLGSNHIRLNSNTYWVYLAPGAYVKGAIEYFTKQNFYATGHGVLSGEN | |
| YVYQANAGDNYVAVKSDSTSLRMWWHNNLGGGQTWYCVGPTINAPPFNTMDFNGNSGI | |
| SQISDYKQVGAFFFQTDGPEIYPNSVVHDVFWHVNDDAIKIYYSGASVSRATIWKCH | |
| NDPIIQMGWTSRDISGVTIDTLNVIHTRYIKSETVVPSAIIGASPFYASGMSPDSSKS | |
| ISMTVSNVVCEGLCPSLFRITPLQNYKNFVVKNVAFPDGLQTNSIGTGESIIPAASGL | |
| TMGLNISSWTVGGQKVTMENFQANSLGQFNIDGSYWGEWQISRISSSQSA | |
| DNA sequence | |
| (SEQ ID NO: 27) | |
| 1 | atggccacaa tgctaaagct acttgcgttg acccttgcaa ttagcgagtc cgccattgga |
| 61 | gcagtcatgc acccacctgg cgtttctcat cccggtaccc atacgggcac tacgaataat |
| 121 | acccattgcg gcgccgactt ctgtacctgg tggcatgatt caggggagat caacacgcag |
| 181 | acacctgtcc aaccagggaa cgtgcgccaa tctcacaagt attccgtgca agtgagtcta |
| 241 | gctggtacaa acaactttca tgactccttt gtatatgaat cgatcccccg gaacggaaat |
| 301 | ggtcgcatct atgctcccac cgatccatcc aacagcaaca cattagattc aagcgtggat |
| 361 | gatggaatct cgattgagcc tagtatcggc ctcaatatgg catggtccca attcgagtac |
| 421 | agccaggatg tcgatataaa gatcctggca actgatggct catcgttggg ctcaccaagt |
| 481 | gatgttgtta ttcgccccgt ctcaatctcc tatgcgattt ctcagtccaa cgatggcggg |
| 541 | attgtcatcc gggtcccagc cgatgcgaac ggccgcaaat tttcagtcga attcaaaaat |
| 601 | gacctgtaca ctttcctctc tgatggcaac gagtacgtca catcgggagg tagcgtcgtc |
| 661 | ggcgttgagc ctaccaacgc acttgtgatc ttcgcaagtc cgtttcttcc ttctggcatg |
| 721 | attcctcata tgaaacccca caacacgcag accatgacgc caggtcctat caataacggc |
| 781 | gactggggcg ccaagtcaat tctttacttc ccaccaggtg tatactggat gaaccaagat |
| 841 | caatcgggca actcgggtaa attaggatct aatcatatac gtctaaactc gaacacttac |
| 901 | tgggtctacc ttgcccccgg tgcgtacgtg aagggtgcta tagagtattt caccaagcaa |
| 961 | aacttctatg caactggtca tggtgtccta tcaggtgaaa actatgttta ccaagccaat |
| 1021 | gctggcgaca actatgttgc agtcaagagc gattcgacca gcctccggat gtggtggcac |
| 1081 | aataaccttg ggggtggtca aacatggtac tgcgttggcc cgacgatcaa tgcgccacca |
| 1141 | ttcaacacta tggatttcaa tggaaattct ggcatctcaa gtcaaattag cgactataag |
| 1201 | caggtgggag ccttcttctt ccagacggat ggaccagaaa tctatcccaa tagtgtcgtg |
| 1261 | cacgacgtct tctggcacgt caatgatgat gcaatcaaaa tctactattc gggagcatct |
| 1321 | gtatcgcggg caacgatctg gaaatgtcac aatgacccaa tcatccagat gggatggaca |
| 1381 | tctcgggata tcagtggagt gacaatcgac acattaaatg ttattcacac ccgctacatc |
| 1441 | aaatcggaga cggtggtgcc ttcggctatc attggggcct ctccattcta tgcaagtggg |
| 1501 | atgagtcccg attcaagcaa gtccatatcc atgacggttt caaacgttgt ttgcgagggt |
| 1561 | ctttgcccgt ccctgttccg catcacaccc ctacagaact acaaaaattt tgttgtcaaa |
| 1621 | aatgtggctt tcccagatgg gctacagaca aatagtattg gcacaggaga aagcattatt |
| 1681 | ccagccgcat ctggtctaac gatgggacta aatatctcca gctggactgt tggtggacaa |
| 1741 | aaagtgacaa tggagaactt tcaagccaat agcctggggc agttcaatat tgacggcagc |
| 1801 | tattgggggg agtggcagat tagtcgaatt tccagctctc agagcgcgtg agtgcttcta |
| 1861 | cccgctcctt tacccttgtc gaaggatcaa ggcataagtt agctcatgtg aaggcgattt |
| 1921 | cagttcattc tctctttttt ggagctcatt tccttttcga ccaattgtga caccaaattg |
| 1981 | ccatgtgtac tgtaattggt caaatgaacg ttaaccttcg atttaatatg gacatttcca |
| 2041 | ggtttcctta ctctgtcgat tatgcctaac tcgggttgat gtcttgtcag gatgaaaatc |
| 2101 | tcgttgtcat gtacttcgag tgaaatgggc agggctaacc cctaagccct aacgcccaat |
| 2161 | cgacttataa gtctagatgt ttatgctatg caggctctgg aatgatttac attccatgct |
| 2221 | ataca |

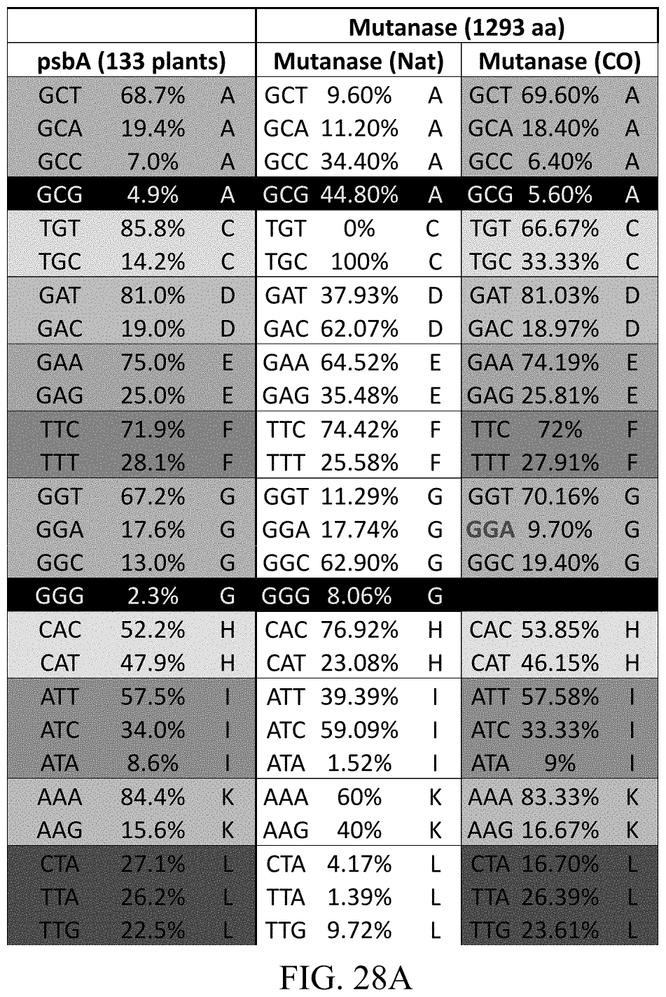

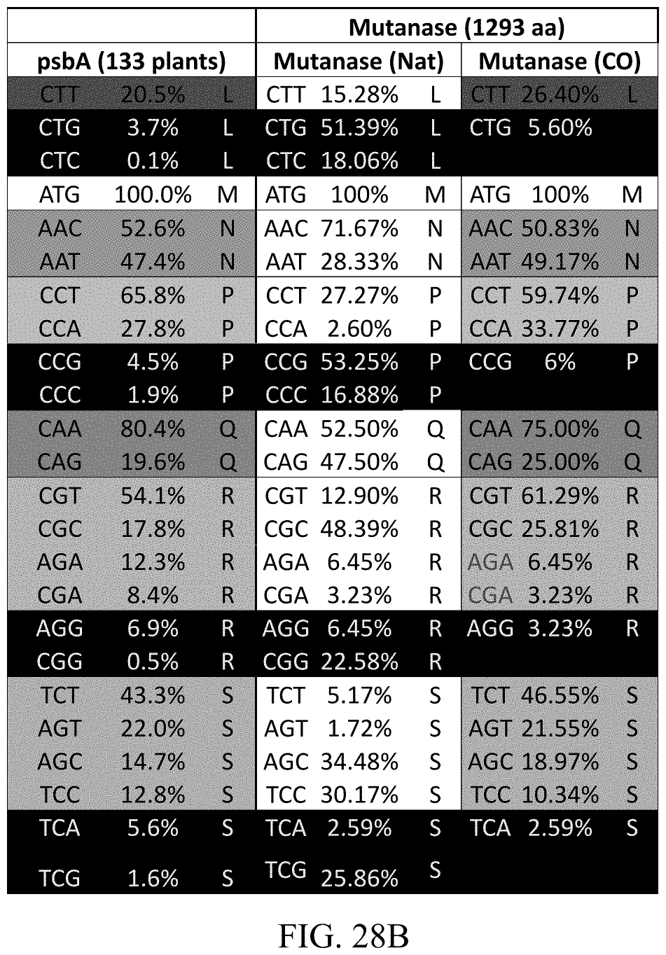

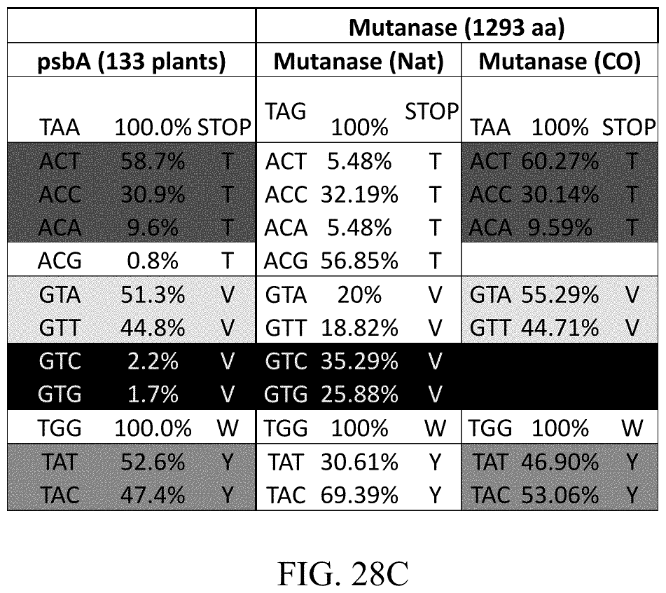

The amino acid and nucleic acid sequence of the triacylglycerol lipase and other information regarding lipY can be found on the UNIPROT website on the world wide web at uniport.org/unitpro/I6Y2J4 (SEQ ID NO: 38) and on the NCBI website NCBI Reference Sequence: YP_177924.1. The nucleic acid sequence encoding lipase could optionally be codon optimized for maximal plant plastid expression using the guidance provided in FIG. 28.

```
                10 20 30 40
MVSYVVALPE VMSAAATDVA SIGSVVATAS QGVAGATTTV
                50 60 70 80
LALAEDEVSA AIAALFSGHG QDYQALSAQL AVFHERFVQA
                90 100 110 120
LTGAAKGYAA AELANASLLQ SEFASGIGNG FATIHQEIQR
              130 140 150 160
APTALAAGFT QVPPFAAAQA GIFTGTPSGA AGFDIASLWP
              170 180 190 200
VKPLLSLSAL ETHFAIPNNP LLALIASDIP PLSWFLGNSP
              210 220 230 240
PPLLNSLLGQ TVQYTTYDGM SVVQITPAHP TGEYVVAIHG
              250 260 270 280
GAFILPPSIF HWLNYSVTAY QTGATVQVPI YPLVQEGGTA
              290 300 310 320
GTVVPAMAGL ISTQIAQHGV SNVSVVGDSA GGNLALAAAQ
              330 340 350 360
YMVSQGNPVP SSMVLLSPWL DVGTWQISQA WAGNLAVNDP
              370 380 390 400
LVSPLYGSLN GLPPTYVYSG SLDPLAQQAV VLEHTAVVQG
              410 420 430
APFSFVLAPW QIHDWILLTP WGLLSWPQIN QQLGIAA
```

### Example III

### Dental Biofilm Disruption Using Chloroplast Made Enzymes with Chewing Gum Delivery

Current approaches for oral health care rely on procedures that are unaffordable to impoverished populations. As aerosolized droplets in the dental clinic and poor oral hygiene may contribute to spread of several infectious diseases, including COVID-19, new solutions for dental biofilm/plaque treatment at home are required. In this example, an affordable method for dental biofilm disruption via expression of lipase, dextranase or mutanase in chloroplast vectors in plant cells is described. The antibiotic resistance gene used to for selection of chloroplast genetransformants were subsequently removed using direct repeats flanking the aadA gene and enzymes were successfully expressed in marker-free lettuce transplastomic lines. Equivalent enzyme units of plant-derived lipase performed better than purified commercial enzymes against biofilms, specifically targeting fungal hyphae formation.

Combination of lipase with dextranase and/or mutanase suppressed biofilm development by degrading the biofilm matrix, with concomitant reduction of bacterial and fungal accumulation. In chewing gum tablets formulated with freeze-dried plant cells, expressed protein was stable up to 3 years at ambient temperature and was efficiently released in a time-dependent manner using a mechanical chewing simulator device. Development of edible plant cells expressing enzymes eliminates the need for purification and cold-chain transportation, providing a translatable therapeutic approach. Biofilm disruption through plant enzymes and chewing gum-based delivery offers an effective and affordable dental biofilm control at home particularly for populations with minimal oral care access.

### Materials and Methods

### Codon Optimization of the Mut Gene

A codon usage reference table for codon optimization based on codon usage frequency of psbA gene in 133 plant species was previously developed (Kwon et al., 2016). Native mutanase coding mut gene nucleotide sequence from Paenibacillus sp. was codon optimized by replacing less preferred codons with more preferred ones, which eventually generated codon usage frequency close to that in reference psbA gene. Rare codons in the native mut gene sequence with a frequency <5% in the reference were replaced by more preferred codons (FIG. 28 and FIG. 9A).

PG1-Smdex and Mut Gene (Co) Cloning in pLsLF-Marker-Free Chloroplast Vector

The native Smdex gene sequence from S. mutans (ATCC 25175) fused with PG1 (Protegrin-1 encoding) downstream, and codon optimized mut gene were synthesized (GenScript Biotech, Piscataway Township, NJ) (Liu et al., 2016). The PG1-Smdex and synthesized mut (co) genes were cloned into pLsLF-marker-free vector using NdeI and PshAI restriction sites and transformed into TOP10 E. coli cells. The lettuce chloroplast transformation vector pLsLF, which contains spectinomycin-resistant gene (aadA, aminoglycoside 3′-adenylytransferase gene) as selectable marker, was used as a backbone.

To design a marker-removable vector, the 649-bp long direct repeat DNA sequence, derived from atpB promoter and 5′ UTR (Daniell et al., 2019a,b; Kumari et al., 2019), was PCR amplified using lettuce total genomic DNA as a template. The sequence-confirmed direct repeats were then cloned to flank aadA expression cassette. For the insertion of single-digested atpB fragments into the vector backbone, NEBuilder HiFi DNA (NEB, Ipswich, Mass.) assembly kit was used to avoid the possible ligation of the fragments in a reverse direction.

The successful insertions were confirmed by restriction digestion. Expression of PG1-dextranase and mutanase in E. coli was confirmed by Western blot. The pLsLF-MF-PG1-dextranase and pLsLF-MF-mutanase (co) plasmid were extracted using PureYield™ plasmid Midiprep System (Promega, Madison, Wis.) and used for subsequent particle bombardment.

### Generation and Molecular Characterization of Marker-Free Transplastomic Lettuce Lines

The pLsLF-MF-PG1-dextranase and pLsLF-MF-mutanase (co) plasmids were transformed into 1-month-old lettuce (L. sativa) leaves by bombardment as previously described (Lee et al., 2011). After the bombardment, lettuce leaves were cut into small pieces (<1 cm²) and grown on regeneration media containing spectinomycin (50 mg/mL) as described previously (Daniell et al., 2019a,b; Kumari et al., 2019; Lee et al., 2011; Ruhlman et al., 2010). The integration of pLsLF-MF-PG1-dextranase and pLsLF-MF-mutanase (co) vectors in regenerated shoots were validated by Southern blot and PCR using specific primers sets:

16S-F,
(SEQ ID NO: 29)
5′-CAGCAGCCGCGGTAATACAGAGGATGCAAGC
aadA-R,
(SEQ ID NO: 30)
5′-CCGCGTTGTTTCATCAAGCCTTACGGTCACC;
atpB-R,
(SEQ ID NO: 31)
5′-GAATTAACCGATCGACGTGCTAGCGGACATT;
UTR-F,
(SEQ ID NO: 32)
5′-AGGAGCAATAACGCCCTCTTGATAAAAC
23S-R,
(SEQ ID NO: 33)
5′-TGCACCCCTACCTCCTTTATCACTGAGC

The PCR positive leaves were subjected to the second round of selection on regeneration media containing spectinomycin (50 mg/L). Any regenerated shoots showing bleached leaves were immediately evaluated by PCR analysis to confirm the excision of aadA gene and were then transferred to spectinomycin-free rooting media to induce roots. Once the roots were formed, homoplasy was confirmed by Southern blots as described below. Expression of enzymes in homoplasmic lines were confirmed using Southern blots and enzyme assays as described previously (Daniell et al., 2019a,b; Kumari et al., 2019; Ruhlman et al., 2010; Verma et al., 2008).

### Southern Blotting of Marker-Free Lettuce Plants

For Southern blotting, 2 μg total genomic DNA from untransformed WT, T1 and T2 generation marker-free plants integrated with PG1-Smdex, lipY and T0 generation integrated with mut (co) were digested by suitable restriction enzymes and separated in 0.8% agarose gel, transferred onto the nylon membranes (Nytran, GE Healthcare), and probed as described previously (Kumari et al., 2019; Kwon et al., 2018).

Seeds from untransformed WT and previously developed three independent T0 transplastomic lipase (Kumari et al., 2019) expressing plants were germinated on ½ MS medium without any antibiotics. The germinated seedlings were transferred and grown in magenta box. The genomic DNA from leaves of two different T1 plants in each of the three independent events (in total six plants) and WT was isolated, and digested with SmaI restriction enzyme (New England Biolabs, Hertfordshire, UK).

### Plant-Derived Enzymes Activity Assay

Leaves from marker-free transplastomic plants expressing lipase, PG1-dextranase, mutanase and untransformed WT plants were stored at −80° C. and freeze-dried in a lyophilizer as described previously (Daniell et al., 2019a,b). Lyophilized leaves were ground into fine powder in a blender and used as the source material for the proteins/enzyme extraction. Total soluble proteins (TSP) was extracted by suspending 50 mg of plant powder in 1 ml plant extraction buffer (respective buffer of each enzymes and EDTA-free protease inhibitor cocktail (Thermo Scientific, Waltham, Mass., USA) and kept on a mixer (Eppendorf) at 4° C. for 1 h. Samples were sonicated, centrifuged at 9391 g for 30 min and the supernatant (TSP) was collected. TSP was quantified by Bio-Rad protein assay dye (Bio-Rad, Hercules, Calif., USA) by following the manufacturer's protocol. Bovine serum albumin (BSA) protein was used as standard. The impact of sonication during extraction of TSP and the role of PIC in the extraction buffer was evaluated by independent experiments. The stability of recombinant enzyme in each crude extract was evaluated by activity assay and compared.

### Dextranase Assay

Leaves harvested at two different time points (30 and 45 days) from two independent marker-free PG1-dextranase transplastomic events (Plant-46 and Plant-47) were lyophilized, ground into powder and evaluated for the enzyme activity. Blue dextran plate assay was performed for qualitative enzyme activity analysis. Blue dextran substrate (from Leuconostoc mesenteroides, Sigma, Louise, Mo., USA) and agar were added into 100 m_{M} sodium acetate buffer (pH 5.5) at final concentration of 0.5% and 1.25% respectively. The suspension was boiled, mixed properly and poured into the plate. After solidification, small wells were created and 50 μg of plant crude extract (TSP) from PG1-dextranase transplastomic plants and WT plant (as negative control) was loaded into the wells. The plate was incubated at 37° C. overnight. Enzymatic activity of PG1-dextranase was visualized in the form of a halo caused by the breakdown of blue dextran around the well. Purified dextranase enzyme from Penicillium sp. (Sigma) was used as positive control.

An enzyme assay was performed to quantify dextranase activity in the crude extract of transplastomic PG1-dextranase plants. TSP of PG1-dextranase transplastomic and WT plants (50 μL) was incubated with 50 μL of dextranase (1% in 100 mm sodium acetate pH 5.5), incubated at 37° C. for 1 h and the released sugar was estimated by dinitrosilycilic acid method (Kumari et al., 2019) using maltose as standards. The enzymatic assay was performed in triplicates and the data are presented as mean and standard deviation. The enzyme activity was represented as sugar released μmol/h/g dry weight of plant powder. The importance of sonication for the release of PG1-dextranase enzyme from the plant powder and PIC for the stability of the released enzyme in the crude plant extract was also evaluated. Plant powder of 45 days old leaves from plant-46 was used for these experiments.

### Lipase Assay

Lipase enzyme assay was performed using the method described previously (Kumari et al., 2019). Briefly, TSP was extracted from the lyophilized plant powder in 100 m_{M} sodium phosphate buffer (pH 8.0) and was quantified. In the assay, 50 μL of TSP and 100 m_{M} p-nitrophenyl butyrate (5 μL) was mixed in 450 μL reaction buffer (100 m_{M} sodium phosphate buffer pH 8 and 0.9% NaCl) and incubated at 37° C. for 10 min. Released p-nitrophenol (hydrolysed product of p-nitrophenyl butyrate) was estimated by using different known concentrations of p-nitrophenol as standards. The enzyme assay was performed in triplicate and data are presented as mean and standard deviation. The enzyme activity was represented as p-nitrophenol released μmol/h/g dry weight. The effect of sonication during the protein extraction and stability of enzyme in the absence of PIC was also examined for plant-derived lipase as described earlier.

### Mutanase Assay

Lyophilized plant powder was extracted with a ratio of 50 mg powder/1 mL extraction buffer [0.1_{M} sodium acetate buffer (pH 5.5)], followed by vortex homogenization at 4° C. for 1 h (Eppendorf 5432) and sonication for 3 cycles (5 s on, 10 s off, 80% amplitude). The homogenate was centrifuged at 9391 g for 30 min and the supernatant was transfer into a new tube. Then the supernatant was dialysed as follows: 10 mL of plant protein extraction was sealed in 15 cm of semi-permeable membrane (MWCO: 20 KD) and placed in 2 L of extraction buffer for 4 h. This buffer was replaced with fresh buffer and incubated at 4° C. overnight (16 h). The total protein concentration was determined by Bradford assay.

The activity of mutanase in the dialysed plant crude was determined by enzymatic assays as described with some modifications (Kopec et al., 1997; Verma et al., 2008). Crude extracts of transplastomic plants were incubated with the substrate (mutan or α-1, 3-linked glucans) in 0.1 M sodium acetate buffer (pH 5.5) at 37° C. for 60 min. The amount of reducing sugar released was determined by the Somogyi-Nelson method (Somogyi, 1945). Crude extracts of untransformed plant (WT) with equal protein concentrations were used to check the endogenous mutanase activity from plant cells. One unit (U) of mutanase activity was defined as the amount of enzyme that releases 1.0 μmol of reducing sugar from mutan per hour at pH 5.5 at 37° C. Mutanase (EC3.2.1.59) purified from Bacillus sp. fermentation (Amano Enzyme, Japan) was used as the commercial enzyme standard.

### Preparation of Chewing Gum and GFP Stability and Release Assay

Chewing gum tablets containing ground plant powder were prepared by compression process. Gum tablets contained the gum base (27.71%), sorbitol (17.18%), maltitol (14.78%), xylitol (13.86%), isomalt (10.07%), natural and artificial flavors (7.21%), magnesium stearate (2.95%), silicon dioxide (0.82%), stevia (0.42%) and plant cell powder (5%) in order to offer the best flavor, taste, softness and compression. The gum tablet chews and performs exactly like the conventional chewing gum based on physical characteristics.

For stability assay, 125 mg of gum tablet was ground with 500 μL protein extraction buffer [(0.2 M Tris HCl pH 8.0, 0.1 M NaCl, 0.01 M EDTA, 0.4 M sucrose, 0.2% Triton X supplemented with 2% PMSF and a PIC (Pierce, Waltham, Mass., USA)], followed by sonication at 80% amplitude for 5 s for two times. After sonication, the samples were centrifuged at 13 523 g at 4° C. for 10 min. Then 100 μL of supernatant was loaded to a fluorescent microplate reader where the GFP was detected at 485 nm (excitation) and 538 nm (emission) using the commercial GFP (Vector Laboratories, San Diego, Calif., USA) as a standard. Release of GFP from gum tablets was studied using a Universal Mechanical Testing Machine equipped with Merlin software (Instron Model 5564, Norwood, Mass.) in cyclic loading mode.

Chewing gum tablets (25 mg) were placed in 10 mL of artificial saliva (Pickering Laboratories, Mountain view, CA) in a polycarbonate chamber and loaded cyclically in compression using a piston attached to a load cell. A load range of −1.5 to −500N for intervals of 1, 5, 7 and 10 min and cycles of 55, 287, 364 and 591 to simulate human chewing. A wide range of bite forces have been reported for adult humans. Values ranging from 1300N to 285N have been reported (Takaki et al., 2014; Yong, 2010). Varga et al. (2011) reported mean bite force values of 522N for males and 465N for females with normal occlusion. A representative compressive load of 500N was selected for this study. The GFP concentrations in both the supernatant and the pellet after 1, 5, 7, and 10 min were determined by the same above-mentioned method.

### Microorganisms Used in Biofilm Studies and Growth Conditions

Candida albicans SC5314, a well-characterized fungal strain and Streptococcus mutans UA159 serotype c (ATCC 700610), a cariogenic dental pathogen and well-characterized EPS producer were used in cross-kingdom biofilm experiments. To prepare the inoculum used in this study, C. albicans (yeast form) and S. mutans cells were grown to mid-exponential phase in ultrafiltered (10-kDa molecular-mass cutoff membrane; Millipore, MA) tryptone-yeast extract broth (UFTYE; 2.5% tryptone and 1.5% yeast extract) with 1% (wt/vol) glucose at 37° C. and 5% CO₂ as described previously (Falsetta et al., 2014).

### In Vitro Biofilm Model and Topical Treatment Regimen

Biofilms were formed using saliva-coated hydroxyapatite (sHA) disc model as described above and by Falsetta et al., 2014 and Hwang et al., 2017. Briefly, the hydroxyapatite discs (surface area, 2.7±0.2 cm²; Clarkson Chromatography Products, Inc., South Williamsport, Pa.) coated with filter-sterilized, clarified whole saliva were vertically suspended in a 24-well plate using a custom-made disc holder (FIG. 19A), mimicking the dental enamel surface. The fungal-bacterial inoculum containing approximately 2×10⁶ CFU/mL of S. mutans and 2×10⁴ CFU/mL of C. albicans (in yeast form) at mid-exponential growth phase in UFTYE (pH 7.0) with 1% (wt/vol) sucrose; this proportion of the microorganisms is similar to that found in saliva samples collected from children with early childhood caries (ECC) (de Carvalho et al., 2006).

Biofilms were maintained at 37° C. under 5% CO₂. To access the antibiofilm efficacy of the plant crude extracts, we developed a topical treatment regimen for feasible clinical applications (FIG. 19B). The sHA surface (a tooth surrogate) was topically treated with plant leaf crude extract for 60 min at 37° C. to mimic the first application after toothbrushing. The sHA disks were inoculated with the culture medium containing the bacterial-fungal inoculum and the mixed biofilms were allowed to initiate under cariogenic (sucrose-rich) conditions for 6 h. The second topical treatment was then performed using the same plant crude extract with 60 min exposure to mimic a repeated application. After that, the treated sHA disks were transferred back to the culture medium for continued biofilm development.

### Microbiological Analysis of the Biofilms

Biofilms were grown until 19 h and were subject to microbiological analyses, including the total number of viable cells (CFU) of bacteria/fungi and the total biomass on each sHA disk (dry weight) as detailed elsewhere (Hwang et al., 2017). Briefly, at 19 h, the biofilms were harvested from the sHA disks and homogenized via optimized sonication procedure, which does not kill fungal or bacterial cells while providing maximum recoverable counts (Koo et al., 2013). Aliquots of biofilm suspensions were serially diluted and plated on blood agar plates and the plates were grown at 37° C. under 5% CO₂ for 2 days. Bacterial and fungal viability in the biofilm was accessed by determining their respective CFU recovered on the blood agar plates. The amount of biofilm dry weight (biomass) was also determined.

### Three-Dimensional Confocal Biofilm Imaging and Quantitative Analysis

The impact of the topical treatments was assessed by examining the 3D architecture and the spatial distribution of Gtf-derived EPS glucans and fungal/bacterial cells within live biofilms using well-established protocols optimized for biofilm imaging and quantification (Falsetta et al., 2014; Hwang et al., 2017). Briefly, the EPS glucan matrix was labelled via incorporation of AlexaFluor 647 dextran conjugate (Molecular Probes Inc., Eugene, Oreg.) throughout the biofilm formation.

S. mutans was stained with Syto 9 (Molecular Probes), while C. albicans cell wall was labeled with Concanavalin A-tetramethyl rhodamine conjugate (Molecular Probes). High-resolution confocal imaging was performed using confocal laser scanning fluorescence microscope (LSM800 with Airyscan, Zeiss, Germany) equipped with a 20×(1.0 numerical aperture) water immersion objective. Each biofilm was scanned at five randomly selected areas, and confocal image series were generated by optical sectioning at each of these positions. Computational analysis of confocal images using the advanced biofilm 3-dimensional analysis tool BiofilmQ (available on the world wide web at: drescherlab.org/data/biofilmQ) was conducted to determine the biovolumes of bacteria, fungi and EPS in order to complement our microbiological analysis (Hartmann et al., 2021). ImageJ (FIJI) was used for post-acquisition image processing and creating 3-dimensional renderings of biofilm architecture (Schindelin et al., 2012).

### In Situ Cell Viability Staining and Imaging

To investigate the impact of the treatments on bacterial/fungal cell viability within the mixed biofilm, in situ cell viability staining was performed and followed by detailed imaging at single-cell resolution using cell membrane integrity as a biomarker for viable cells. TOTO-3 (Molecular Probes), a cell impermeable dimeric cyanine acid dye as a dead cell indicator for both bacterial and fungal cells because of its high affinity for nucleic acids, was used (Chiaraviglio and Kirby, 2014). Thus, when the microbial cells are killed and the plasma membrane integrity are compromised, these probes will enter cells, bind to nucleic acids, and exhibit a strong fluorescence.

Biofilms were stained using the optimized 1μ_{M} of TOTO-3 in 0.9% sodium chloride at 37° C. for 10 min and were counterstained with 0.65μ_{M} SYTO9 (a cell-permeable dye). Concanavalin A-tetramethylrhodamine conjugate was used to label fungal cell wall as described previously (Falsetta et al., 2014). The biofilms were sequentially scanned (488/640 nm lasers for SYTO9/TOTO-3, then 561 nm laser for Concanavalin A-tetramethylrhodamine) and the fluorescence emitted was collected using optimum emission wavelength filters (Zeiss LSM800 confocal microscope with Airyscan). Each biofilm was scanned at least three randomly selected areas. ImageJ FIJI was used for image processing and to create representative multi-channel images (Schindelin et al., 2012).

### Results

### Generation and Characterization of Marker-Free Lettuce Transplastomic Lines Expressing Dextranase, Mutanase, Lipase

The Smdex gene (2574 bp, gene designation from Kim et al., 2011) encoding dextranase was isolated from S. mutans strain ATCC 25175 genomic DNA using PCR (SEQ ID NOs: 34 and 35) and fused to the PG1 (encoding antimicrobial peptide Protegrin-1). The mut gene (3780 bp, gene designation from Otsuka et al., 2015) of Paenibacillus sp. encoding mutanase was codon optimized to improve its translation efficiency in plant chloroplasts based on psbA genes from 133 plant species as described previously (Kwon et al., 2016). Within total 1260 codons of mut gene, 576 codons including 327 rare codons were replaced by more highly preferred codons, resulting in an increased AT content from 44% to 57% (SEQ ID NOs: 36 and 37).

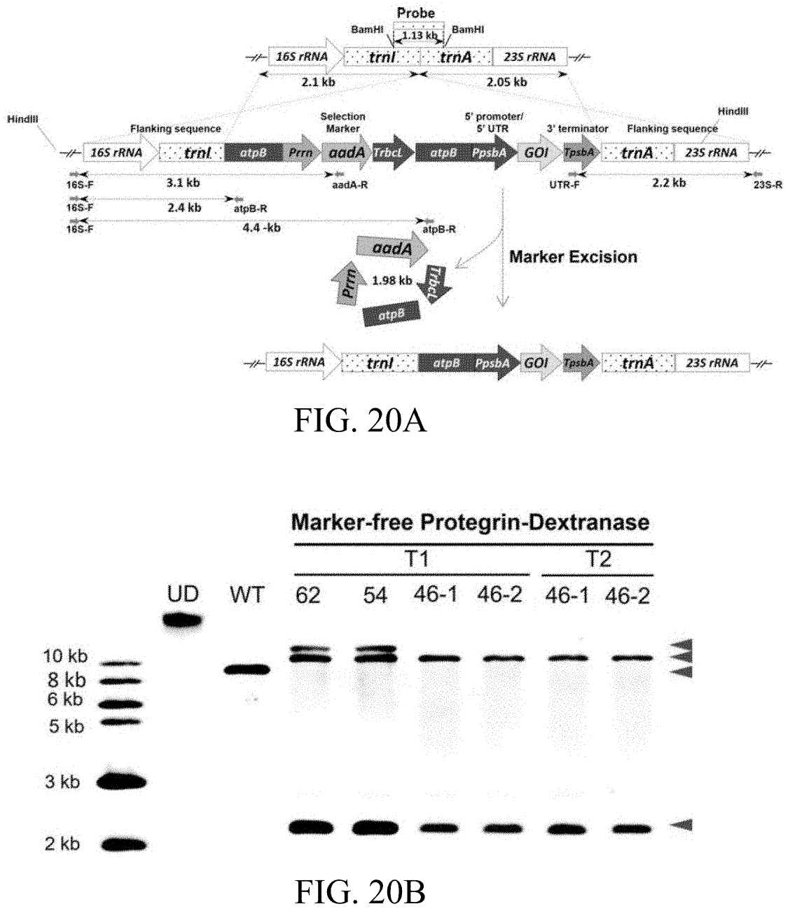

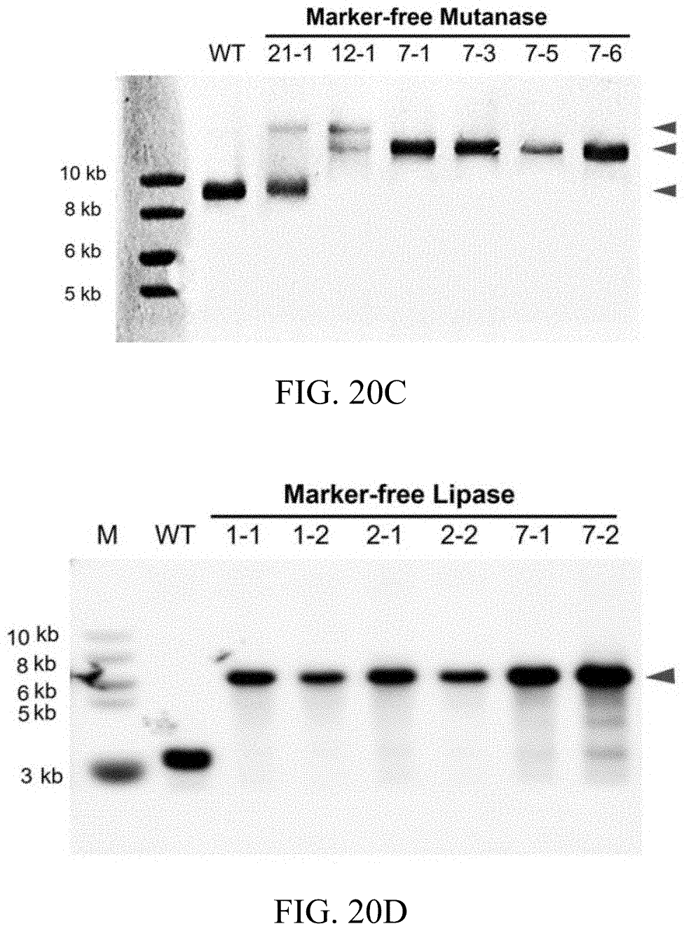

The Smdex, mut and lipY (encoding lipase, gene designation from Deb et al., 2006) genes (native or codon optimized) were cloned into the newly designed marker-free chloroplast vector pLsLF-MF as described previously (Daniell et al., 2019a,b; Daniell et al., 2020; Kumari et al., 2019; Park et al., 2020) and the constructed plasmids were then delivered by gene gun into lettuce (Lactuca sativa) cv. Simpson Elite leaves (Ruhlman et al., 2010). The successful Marker-free events were identified by screening shoots for presence of the transgene cassette but absence of the antibiotic resistance gene aadA by PCR using primers described above (FIG. 20A).

To characterize homoplasmic status of transplastomic lines, total plant gDNA was extracted from marker-free Protegrin-dextranase transplastomic plants, digested with HindIII, and probed with the DIG-labeled trnI and trnA flanking sequence (FIG. 20A). The 9.1 kb hybridizing fragment was only present in the untransformed wild-type (WT) chloroplast genome, but not in the transplastomic lines, confirming their homoplasmic status (FIG. 20B). Therefore, all copies (up to 10,000) of chloroplast genomes had the PG1-Smdex gene cassette stably integrated, within the limits of detection. Two transplastomic lines (54 and 62) showed 10.5 and 12.5 kb bands, indicating a partial marker-free removal process. All other transplastomic lines showed only the 10.5 kb hybridizing fragment, confirming a complete marker removal status.

Most importantly, homoplasmic marker-free PG1-Smdex cassette was stably maintained in T1 and T2 generations of 46-1 and 46-2 lines in the absence of the antibiotic resistance gene (FIG. 20B). The status of T0 transplastomic lettuce plants integrated with marker-free mut construct was confirmed by southern blot using HindIII as well. Some lines (21-1) showed both 16.1 kb and a 9.1 kb fragments suggesting a heteroplasmic status of chloroplast genomes (FIG. 20C) with the marker gene. Additionally, line 12-1 with the 14.1 kb and 16.1 kb bands, but without the 9.1 kb fragment suggested an incomplete removal of the antibiotic resistance gene. The presence of only the 14.1 kb fragment in all other lines confirmed their homoplasmic and marker-free status of all chloroplast genomes (FIG. 20C).

The stability and inheritance of lipY gene in T1 generation and its homoplasmic status was confirmed. The single hybridizing fragment of 5.6 kb in the transplastomic and 3.13 kb in untransformed WT plants after SmaI restriction digestion of gDNA was detected in Southern blot when probed with trnI/trnA genes, flanking the expression cassette (FIG. 20D). The presence of a single larger 5.6 kb hybridizing fragment in all six tested transplastomic lines (compared to the 3.13 kb fragment in WT) confirmed the inheritance and stability of integrated lipY gene and the absence of marker gene in the T1 generation. Moreover, the absence of the 3.13 kb fragment (detected in WT) in each transplastomic plant confirmed their homoplasmic nature.

### Characterization of Plant-Derived Glucanohydrolase and Lipase Activity

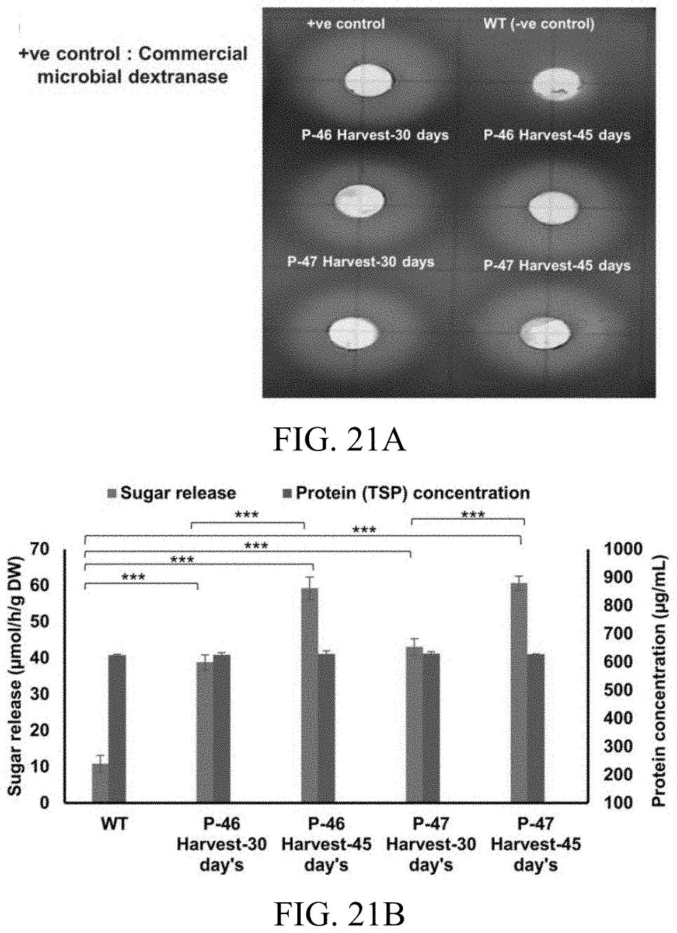

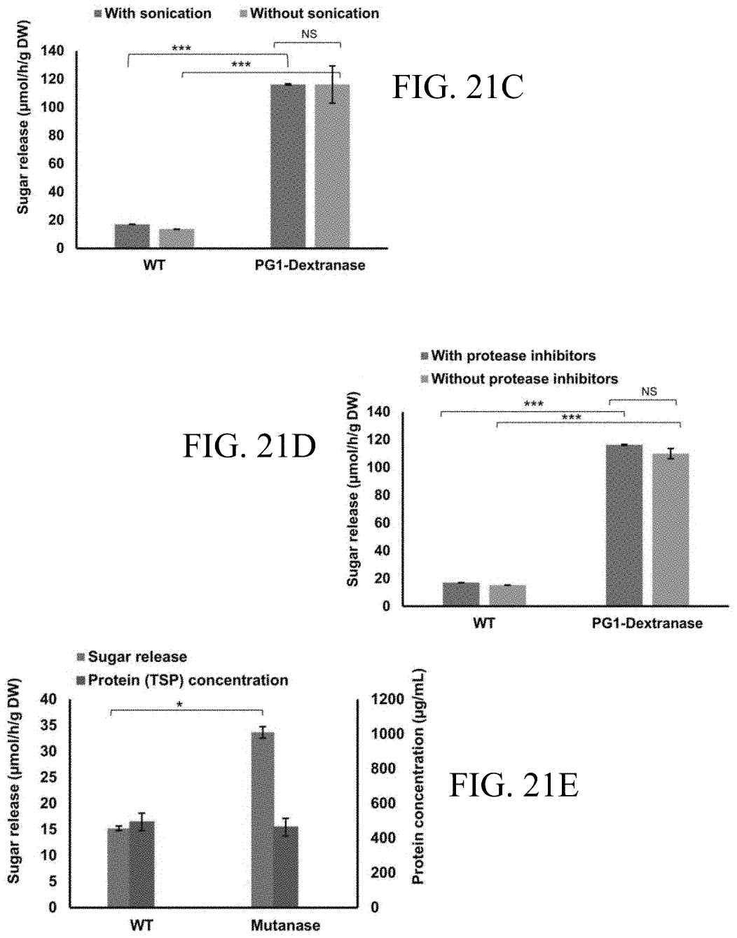

On the plate assay, protein crude extracts from all four tested leaf harvests (30 and 45 days of P-46 and P-47) and the purified commercial dextranase from Penicillium sp. (positive control) produced halo rings on blue dextran, while no halo formation was observed from untransformed WT plant extracts (FIG. 21A). Expression level correlated with the maturity of leaves. In the quantitative assay, dextranase activity evaluated in transplastomic lines P-46 and P-47 at different stages of their growth (30 and 45 days) varied from 38.80±2.04 to 59.24±3.13 and 43.05±2.32 to 60.68±1.91 μmol/h/g dry weight, respectively, confirming again increased expression as leaves matured (FIG. 21B).

Enzyme release from freeze-dried plant cells with or without sonication, showed similar enzyme activity from both preparations (FIG. 21C), thereby eliminating the requirement for sonication for the release of protein from the plant powder, an important criterion for easy release of proteins from chewing gums described below. Similar levels of enzyme activity were observed in the plant extracts with or without protease inhibitor cocktail (PIC) used at the time of protein extraction (FIG. 21D), suggesting that dextranase was resistant to proteases released in the plant crude extract during protein extraction. Statistical significance analysed by t-test for dextranase enzyme activity was P<0.001 (***). The calculated mutanase activity in mature leaves of transplastomic and untransformed WT lettuce were 33.68±1.09 and 15.22±0.43 μmol/h/g dry weight, respectively and statistical significance of mutanase was P<0.05 (*, FIG. 21E). Lower level of mutanase activity than dextranase is probably due to low level of expression in T0 generation but this typically increases 10-20-fold in subsequent generations of transplastomic lines (Park et al., 2020).

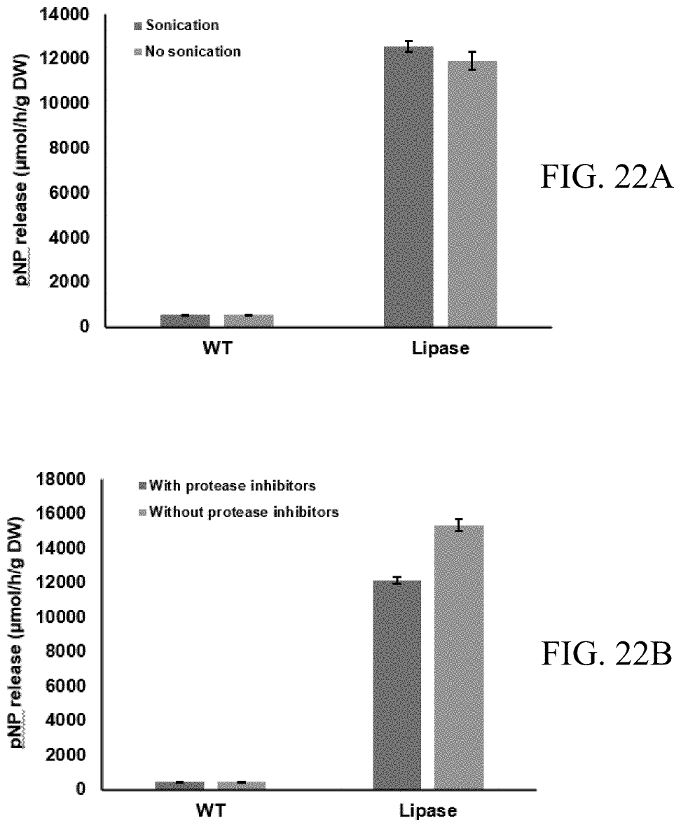

Lipase activities in matured leaves of the transplastomic line and untransformed WT were 12 542.52±257.03 and 522.76±12.85 μmol/h/g dry weight, respectively with sonication and PIC in the extraction buffer. Lipase extracted without sonication but with PIC showed almost the same level of activity (FIG. 22A), indicating that the ultrasonic disruption was not required for enzyme release, making this suitable for the chewing gum approach. Interestingly, proteins extracted in the absence of PIC showed a 21% increase in the enzyme activity (FIG. 22B).

### Antibiofilm Activity of Plant Derived and Purified Commercial Lipase

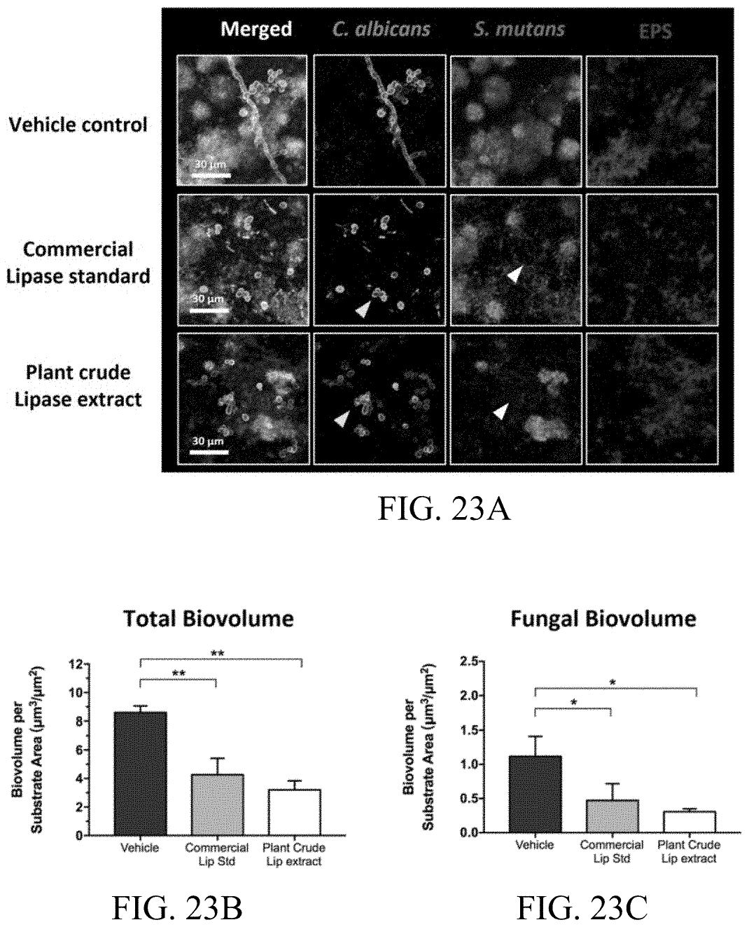

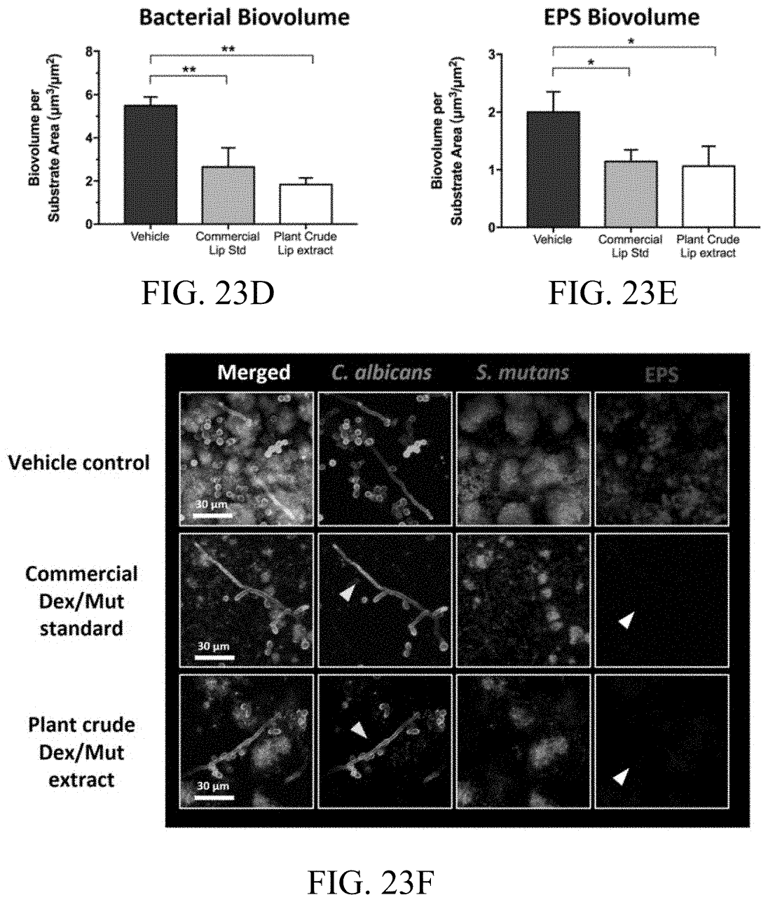

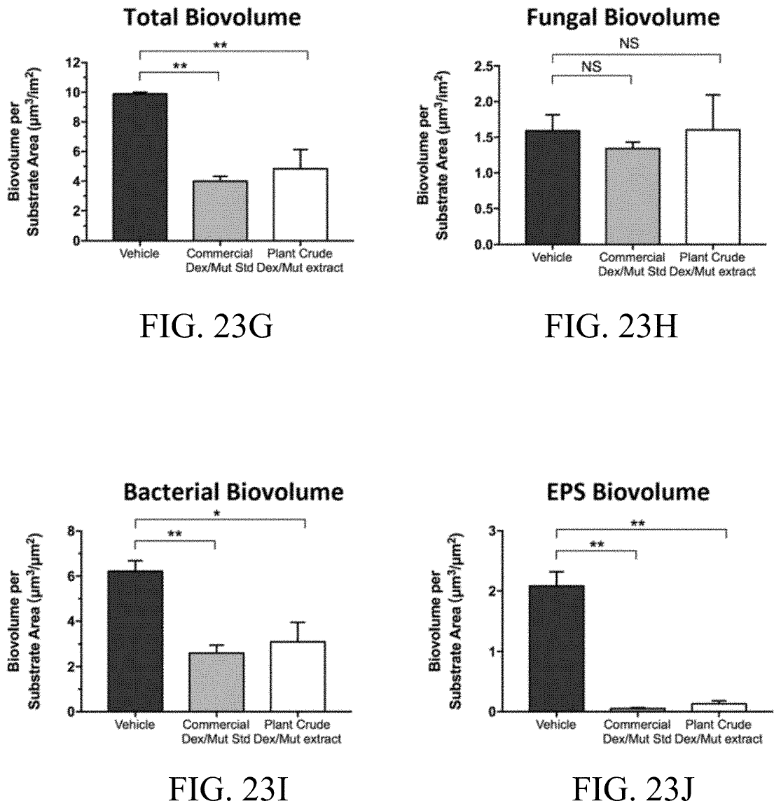

Efficacy of the plant-derived lipase was evaluated by employing a mixed-kingdom biofilm model and a treatment regimen based on topical exposure (FIG. 19). Purified commercial lipase of equivalent enzyme activity unit was tested as positive control. The data revealed that treatment with plant-lipase extract or purified lipase significantly inhibited Candida (C. albicans) hyphal formation, a key factor for cross-kingdom interaction and biofilm development, and reduced bacterial (S. mutans) and EPS glucans (EPS) accumulation (FIG. 23A). Fungal cells were mostly in yeast form (FIG. 23A, shaded arrow heads) with less bacterial clusters and more dispersed cells (white arrow heads), whereas the EPS matrix formation was also disrupted.

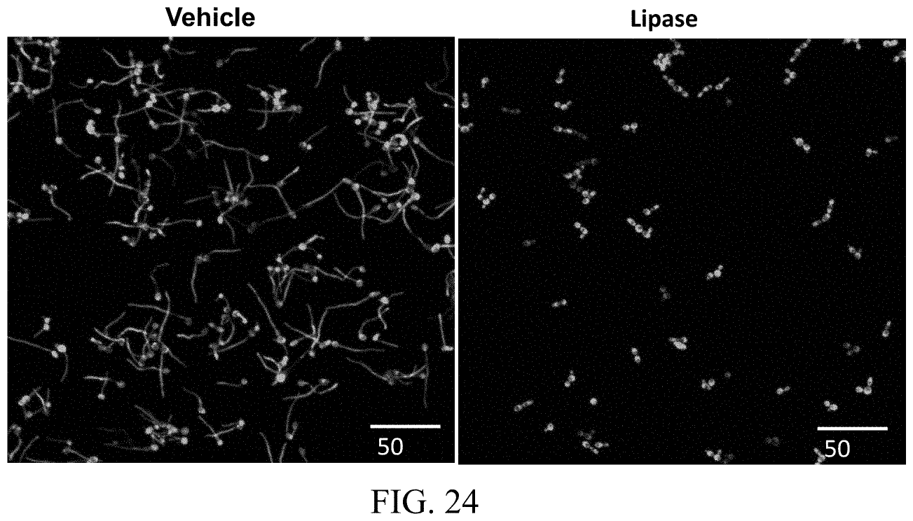

Quantitative computational analysis was also performed using the images acquired via confocal microscopy. The plant-lipase crude extract significantly reduced the total biovolume of the mixed biofilm (FIG. 23B, >50% reduction compared to vehicle-control, P<0.01). Further analysis of each fluorescence channel revealed reduction of both the fungal (FIG. 23C, P<0.05) and bacterial biovolume (FIG. 23D, P<0.05). This analysis was consistent with the confocal images (FIG. 23A). Notably, the plant-lipase extract was as effective as or more effective than the purified commercial lipase in disrupting biofilms. The total amount of EPS glucan was significantly reduced (FIG. 23E, P<0.05) in the treated biofilms. Altogether, the data indicates that the plant-lipase extract potently inhibits fungal filamentation (a novel finding) that reduced total biovolume of the mixed biofilm. This revealed the plant-lipase extract's potential to replace the commercial purified enzyme based on antibiofilm efficacy. Lipase ability to inhibit C. albicans hyphal formation was evaluated in C. albicans monoculture as well. As expected, similar findings were obtained (FIG. 24).

### Antibiofilm Activity of Plant-Derived and Commercial Purified Dextranase and Mutanase

To investigate whether the plant-derived glucanohydrolases could be used as antibiofilm therapeutics, the bioassay was performed using a mixture of dextranase and mutanase (5:1 activity ratio) that was optimized previously using purified enzymes to provide maximum matrix-degrading activity (Ren et al., 2019). Commercial purified dextranase and mutanase (at the same 5:1 ratio) was used as positive-control. The data showed that both plant-dextranase/mutanase extract and the equivalent purified enzymes were highly effective in disrupting EPS glucans, resulting in near abrogation of glucan-matrix in the mixed-kingdom biofilm (FIG. 23F, white arrow heads). Treatment with the dual-enzyme formulation also resulted in less bacterial clusters accompanied by cellular dispersion (FIG. 23F, S. mutans), thereby reducing the density of bacterial accumulation. However, topical exposure of plant-derived or commercial dextranase/mutanase showed no effects on fungal hyphal production (FIG. 23F, shaded arrow heads).

Further computational analysis confirmed the inhibitory effects exerted by glucanohydrolases. As expected, the EPS was effectively degraded compared to vehicle control (>90% reduction, P<0.01; FIG. 23J). Bacterial accumulation and overall biofilm volume were also significantly reduced in biofilms treated with commercial purified or plant-derived glucanohydrolases (P<0.01; FIG. 23I and P<0.01; FIG. 23G). In contrast, Candida was minimally affected after the treatment by both enzyme preparations (P>0.05; FIG. 23H), consistent with limited effects on fungal hyphal formation. The data indicates that plant-derived dextranase/mutanase can effectively disrupt EPS glucan matrix with equivalent potency to that of commercial enzymes. However, these glucanohydrolases display limited effects on fungal accumulation, indicating that combination with lipase results in a more effective multitargeted approach against mixed-kingdom biofilms.

### Cumulative Effect of Dextranase/Mutanase and Lipase on Biofilm Accumulation

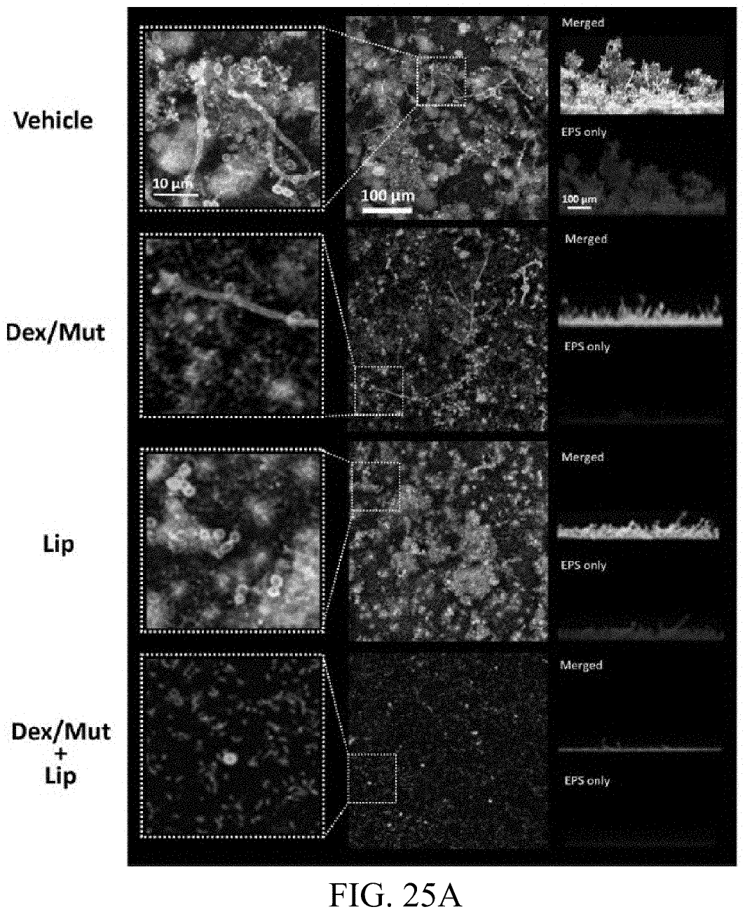

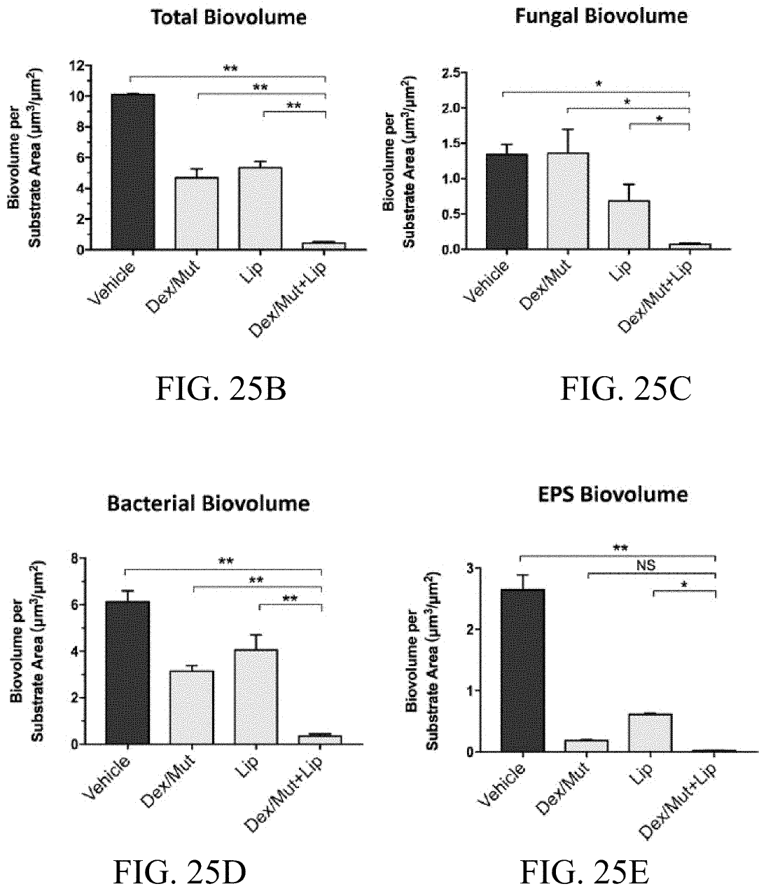

To develop a therapeutic solution for fungal-bacterial mixed biofilms, a combinatorial approach using dextranase/mutanase and lipase to enhance the antibiofilm efficacy was employed. The data show that the topical treatment with dextranase/mutanase and lipase is remarkably effective, resulting in near-complete suppression of mixed-biofilm formation (FIG. 25A). The multi-enzymatic activity eliminated bacterial clustering with few dispersed S. mutans (predominantly single cells) and minimal EPS glucan matrix (FIG. 25A, magnified view in left panel). Notably, few C. albicans yeast cells were attached on the surface whereas hyphal formation was abrogated (FIG. 25A). Quantitative computational analysis confirmed the potent inhibition of fungal/bacterial and EPS biovolumes by the multi-enzyme treatment (FIG. 25B-25E).

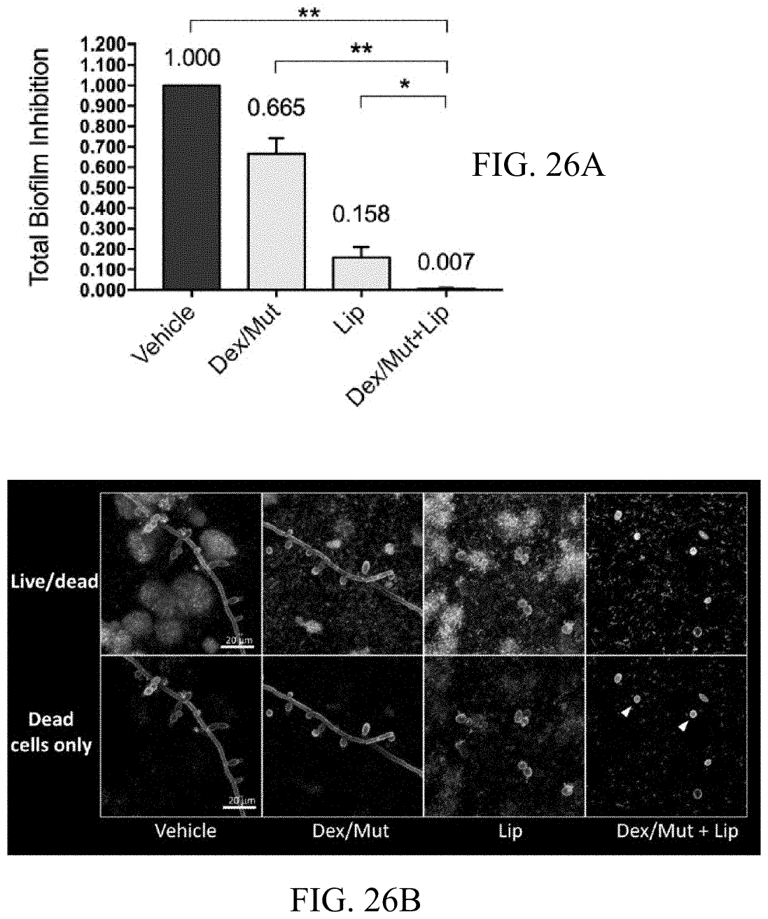

To further investigate the impact of the treatment on the biofilm accumulation, the Total Biofilm Inhibition (TBI) index (FIG. 26A), which evaluates the combined net effects on the reduction of fungal colony forming unit (CFU), bacterial CFU and biomass (dry-weight), was determined. The combination treatment (dextranase/mutanase and lipase) resulted in significantly lower TBI (0.007±0.003) than either dextranase/mutanase (0.665±0.070) or lipase alone (0.158±0.050) (FIG. 26A), indicating synergistic inhibitory effects. Cell viability was also assessed via in situ confocal imaging and fluorescent labeling of live and dead bacterial/fungal cells within intact mixed-kingdom biofilms. Interestingly, in addition to dispersion of bacterial clusters and inhibition of fungal hyphae (FIG. 26B, upper panel), the biofilms treated with dextranase/mutanase and lipase harbored mostly dead C. albicans in yeast form (FIG. 26B, lower panel, white arrow heads). Altogether, the data demonstrates a feasible approach using EPS-degrading enzymes to potently disrupt mixed-kingdom biofilms by reducing both the microbes and matrix components.

### Chewing Gums, Protein Stability and Functionality

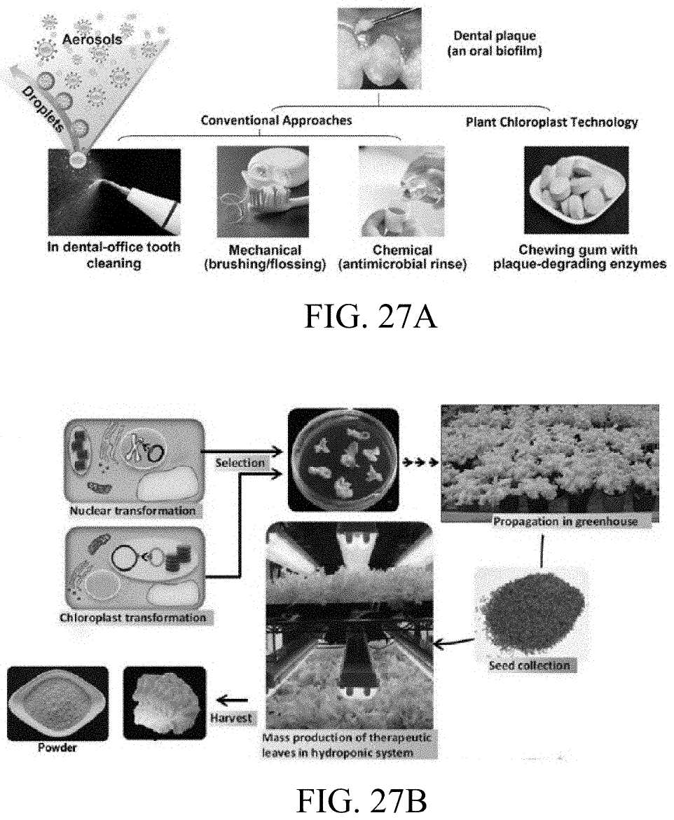

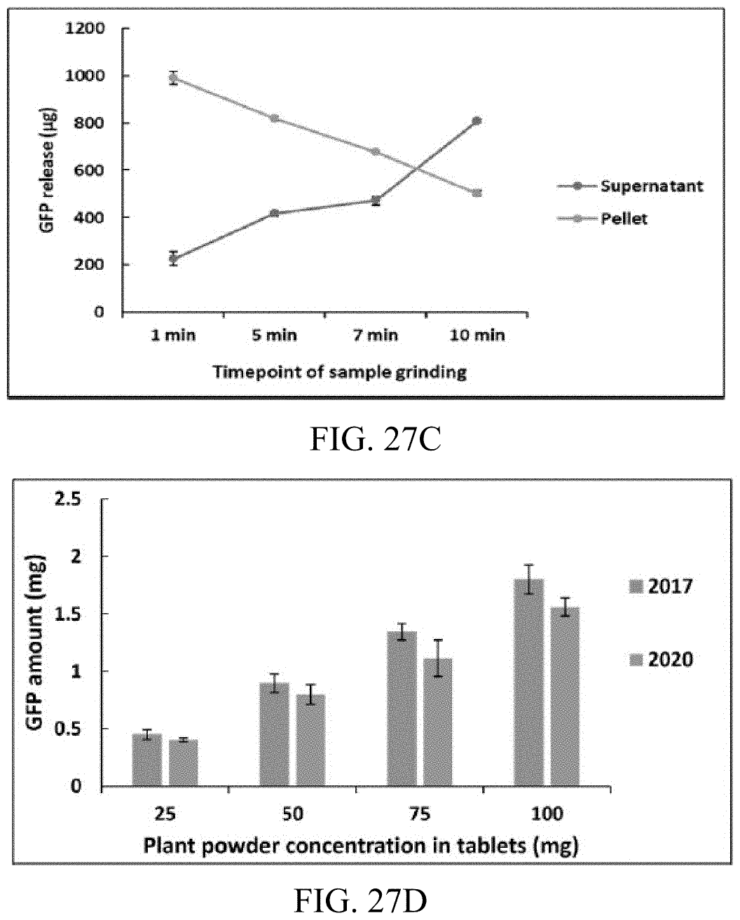

The feasibility of incorporating the plant-derived proteins in chewing gum was tested as an alternative, easy-to-use and more affordable delivery approach (FIG. 27A-27B). To study the release of proteins impregnated in chewing gums, plant cells expressing GFP in ground powder form after lyophilization were utilized (Gupta et al., 2015; Lee et al., 2011). Transplastomic lettuce expressing GFP-protegrin was grown in a Fraunhofer cGMP hydroponic facility, lyophilized and powdered as described previously (Daniell et al., 2019a,b; Daniell et al., 2020; Kumari et al., 2019; Park et al., 2020; Su et al., 2015). Chewing gum tablets using ground plant powder were made through a compression process. The compression process is advantageous over traditional gum manufacturing process which requires higher temperature (93° C.) and extrusion/rolling that introduces variability in the concentration of the active ingredient. Gum tablets performs exactly like conventional chewing gum based on taste, softness and compression.

There was no detectable loss of protein in gum tablets preparation process based on GFP quantification (FIG. 14 and Table 3).

TABLE 3
Chewing Gum tablet preparation

| | Lyophilized powder | | | |
|---|---|---|---|---|
| | 25 mg | 50 mg | 75 mg | 100 mg |
| | GFP released | | | |
| | 0.45 mg | 0.9 mg | 1.35 mg | 1.8 mg |
| Total Weight of the tablet | −2 g | −2 g | −2 g | −2 g |
| Weight of Sample taken | 250 mg | 250 mg | 250 mg | 250 mg |
| for analysis | | | | |
| and GFP concentration | (56 μg) | (112 μg) | (168 μg) | (225 μg) |
| Expected GFP | 448 μg = | 896 ug = | 1344 ug = | 1800 ug = |
| amount/tablet (−2 g) | 0.448 mg | 0.896 mg | 1.344 mg | 1.8 mg |

Release of GFP from gum tablets was studied using a Universal Mechanical Testing Machine by placing chewing gum pellets in 10 mL of artificial saliva in a polycarbonate chamber and loaded cyclically in compression using a piston attached to a load cell. GFP-protegrin concentration in saliva increased from 225 μg/mL in 1 min to 809 μg/mL in 10 min in the supernatant and decreased from 988 μg/mL at 1 min in the pellet to 502 μg/mL at 10 min, confirming steady release during the chewing process (FIG. 27C).

Quantification of GFP in gum tablets showed that GFP was stable in gum tablets when stored at ambient temperature for 3 years (FIG. 27D), indicating a potential long shelf life of the final product.

### DISCUSSION

In order to address the high cost of production and delivery of recombinant therapeutic proteins, protein drugs (PDs) have been expressed in plant chloroplasts and orally deliver them through bioencapsulation within plant cells (Daniell et al., 2016; Daniell et al., 2021; Daniell et al., 2019a,b). Thin lettuce leaves facilitate rapid removal of water through lyophilization and offer an ideal system for expression and delivery of PDs. This platform has advanced to deliver therapeutic proteins in the clinic, yet it remains unexplored in dental medicine. Mechanical disruption, using manual or electric toothbrushes, can remove dental plaque, but they are cumbersome to use, have low compliance, and are costly. Furthermore, current antimicrobial agents to treat cariogenic plaque are inefficient due to the presence of the EPS matrix (Autio-Gold, 2008; Koo et al., 2017; Ren et al., 2019).

Matrix-degrading enzymes can effectively target the biofilm structure and enhance antimicrobial killing, while simultaneously weakening its mechanical stability, thereby promoting bacterial removal (Autio-Gold, 2008; Koo et al., 2017; Ren et al., 2019). However, the development of clinically feasible approaches is hindered by high costs for mass production as the enzymes require complex microbial fermentation and expensive purification procedures.

Here, we describe the expression of mutanase, dextranase and lipase in plant cells and synergistic efficacy when combined with plant-derived lipase for oral biofilm prevention. Although microbial dextranase has been extensively used in food industry with demonstrated safety (Purushe et al., 2012), dextranase has never been expressed in plant cells. Importantly, creation of marker-free edible plant cells expressing enzymes with high yield and stability eliminates the need for purification, providing a translatable therapeutic approach.

Current chemical modalities to treat biofilm-associated infections are primarily targeting individual bacterial or fungal components despite the importance of the extracellular EPS matrix in biofilm antimicrobial tolerance (Koo et al., 2017). In addition, targeting EPS can also disrupt the viscoelastic properties of biofilms and further weaken its mechanical stability, promoting bacterial dispersal or removal (Ren et al., 2019). The data presented herein shows that the lipase, dextranase and mutanase crude plant extracts have potent antibiofilm efficacy that can replace the costly purified enzyme standards of equivalent enzyme units. Although lipase has been approved by FDA to treat several metabolic disorders it remains underexplored to treat dental plaque.

In the present study, lipase remarkably reduced fungal load within treated biofilm by abrogating hyphal formation. This a crucial step in cross-kingdom biofilm development as hyphae provide a scaffold for co-species adhesion and EPS accumulation (Prabhawathi et al., 2014). The precise role of lipids in biofilm formation remains unclear, but recent data suggest that lipids appear to be important membrane components modulating fungal morphogenesis and hyphal elongation (Rella et al., 2016). Hence, the data presented herein indicates that lipase treatment inhibits the filamentous Candida growth impacting cross-kingdom biofilm scaffolding and accumulation. Additionally, plant-derived dextranase and mutanase degraded EPS glucan effectively inhibiting the matrix development and bacterial accumulation. However, after treatment by dextranse and mutanase, fungal cells were unaffected and detectable EPS was still present.

Interestingly, lipase has been shown to have antimicrobial properties possibly through degradation of the membrane lipids (Prabhawathi et al., 2014; Seghal Kiran et al., 2014). Using high-resolution confocal microscopy, we found that the combination of glucanohydrolases and lipase displayed enhanced antimicrobial activity against both species in the biofilms. The data also revealed that degradation of EPS glucans by dextranase and mutanase can locally expose the embedded S. mutans and C. albicans cells within the treated biofilm. This indicates that dextranase and mutanse provide access for lipase to the fungal/bacterial cell surface thereby causing microbial death. Thus, the three-enzyme combination treatment suppressed the pathogenic fungal-bacterial biofilm formation via a complementary mechanism between the effective EPS degradation by glucanohydrolases and the antimicrobial effects of lipase in situ, thereby potentiating antibiofilm efficacy.

The use of chewing gum supplemented with antimicrobial and antibiofilm therapeutics to improve treatments of dental diseases has been proposed as a promising alternative in oral health care applications (Wessel et al., 2016). Yet, no such products are currently available due to high costs for inclusion of therapeutic additives and practical formulation development.

Data presented herein indicate that the combination of dextranase and mutanase at specific amounts and ratios (as employed here) is required to maximize EPS degradation and achieve biofilm disruption. We also demonstrate that the addition of lipase-mediated antimicrobial effects to the dextranase and mutanase combinations resulted in potentiation of effective biofilm eradication.

Here, we demonstrated the feasibility of using chewing gum to release plant-derived proteins in saliva solution following mechanical bite forces. Furthermore, the proteins were stable in chewing gum stored at ambient temperature (up to 3 years), indicating a practical and easy-to-use topical delivery platform. Altogether, we provide a conceptual framework for plant made biofilm-degrading enzymes and chewing gum-based protein delivery as an innovative and affordable approach for controlling dental biofilm. This strategy helps address the societal issue of inequity in access dental care services by enabling a low-cost technology for improvement of oral health, which is also relevant during the current global crisis caused by COVID-19.

### REFERENCES

Autio-Gold, J. (2008) The role of chlorhexidine in caries prevention. Oper. Dent. 33, 710-716.
Bennett, A. M., Fulford, M. R., Walker, J. T., Bradshaw, D. J., Martin, M. V. and Marsh, P. D. (2000) Microbial aerosols in general dental practice. Br. Dent. J. 189, 664-667.
Bowen, W. H., Burne, R. A., Wu, H. and Koo, H. (2018) Oral biofilms: pathogens, matrix, and polymicrobial interactions in microenvironments. Trends Microbiol. 26, 229-242.
de Carvalho, F. G., Silva, D. S., Hebling, J., Spolidorio, L. C. and Spolidorio, D. M. (2006) Presence of mutans streptococci and Candida spp. in dental plaque/dentine of carious teeth and early childhood caries. Arch. Oral Biol. 51, 1024-1028.
Chiaraviglio, L. and Kirby, J. E. (2014) Evaluation of impermeant, DNA-binding dye fluorescence as a real-time readout of eukaryotic cell toxicity in a high throughput screening format. Assay Drug Dev. Technol. 12, 219-228.
Daniell, H., Chan, H. T. and Pasoreck, E. K. (2016) Vaccination via chloroplast genetics: affordable protein drugs for the prevention and treatment of inherited or infectious human diseases. Annu. Rev. Genet. 50, 595-618.
Daniell, H., Jin, S. X., Zhu, X. G., Gitzendanner, M. A., Soltis, D. E. and Soltis, P. S. (2021) Green giant—a tiny chloroplast genome with mighty power to produce high-value proteins: history and phylogeny. Plant Biotechnol. J. 19, 430-447.
Daniell, H., Kulis, M. and Herzog, R. W. (2019a) Plant cell-made protein antigens for induction of oral tolerance. Biotechnol. Adv. 37, 107413. 10.1016/j.biotechadv.2019.06.012.
Daniell, H., Ribeiro, T., Lin, S., Saha, P., McMichael, C., Chowdhary, R. and Agarwal, A. (2019b) Validation of leaf and microbial pectinases: commercial launching of a new platform technology. Plant Biotechnol. J. 17, 1154-1166.
Daniell, H., Mangu, V., Yakubov, B., Park, J., Habibi, P., Shi, Y., Gonnella, P. A. et al. (2020) Investigational new drug enabling angiotensin oral-delivery studies to attenuate pulmonary hypertension. Biomaterials, 233, 119750. 10.1016/j.biomaterials.2019.119750.
Deb, C., Daniel, J., Sirakova, T. D., Abomoelak, B., Dubey, V. S. and Kolattukudy, P. E. (2006) A novel lipase belonging to the hormone-sensitive lipase family induced under starvation to utilize stored triacylglycerol in Mycobacterium tuberculosis. J. Biol. Chem. 281, 3866-3875.
Falsetta, M. L., Klein, M. I., Colonne, P. M., Scott-Anne, K., Gregoire, S., Pai, C. H., Gonzalez-Begne, M. et al. (2014) Symbiotic relationship between Streptococcus mutans and Candida albicans synergizes virulence of plaque biofilms in vivo. Infect. Immun. 82, 1968-1981.
Gupta, K., Kotian, A., Subramanian, H., Daniell, H. and Ali, H. (2015) Activation of human mast cells by retrocyclin and protegrin highlight their immunomodulatory and antimicrobial properties. Oncotarget, 6, 28573.
Hajishengallis, E., Parsaei, Y., Klein, M. I. and Koo, H. (2017) Advances in the microbial etiology and pathogenesis of early childhood caries. Mol. Oral Microbiol. 32, 24-34.
Hartmann, R., Jeckel, H., Jelli, E., Singh, P. K., Vaidya, S., Bayer, M., Rode, D. K. et al. (2021) Quantitative image analysis of microbial communities with BiofilmQ. Nat. Microbiol. 6, 151-156.
Hwang, G., Liu, Y., Kim, D., Li, Y., Krysan, D. J. and Koo, H. (2017) Candida albicans mannans mediate Streptococcus mutans exoenzyme GtfB binding to modulate cross-kingdom biofilm development in vivo. PLoS Pathog. 13, e1006407. 10.1371/journal.ppat.1006407
Kim, Y. M., Shimizu, R., Nakai, H., Mori, H., Okuyama, M., Kang, M. S., Fujimoto, Z. et al. (2011) Truncation of N- and C-terminal regions of Streptococcus mutans dextranase enhances catalytic activity. Appl. Microbiol. Biotechnol. 91, 329-339.
Kolahi, J. and Abrishami, M. (2013) Mutanase-containing chewing gum: a new potential approach for prevention of dental caries. Dental Hypotheses, 4, 53-54.
Koo, H., Allan, R. N., Howlin, R. P., Stoodley, P. and Hall-Stoodley, L. (2017) Targeting microbial biofilms: current and prospective therapeutic strategies. Nat. Rev. Microbiol. 15, 740-755.
Koo, H., Hayacibara, M. F., Schobel, B. D., Cury, J. A., Rosalen, P. L., Park, Y. K., Vacca-Smith, A. M. et al. (2013) Inhibition of Streptococcus mutans biofilm accumulation and polysaccharide production by apigenin and tt-farnesol. J. Antimicrob. Chemother. 52, 782-789.
Kopec, L. K., Vacca-Smith, A. M. and Bowen, W. H. (1997) Structural aspects of glucans formed in solution and on the surface of hydroxyapatite. Glycobiology, 7, 929-934.
Kumari, U., Singh, R., Ray, T., Rana, S., Saha, P., Malhotra, K. and Daniell, H. (2019) Validation of leaf enzymes in the detergent and textile industries: launching of a new platform technology. Plant Biotechnol. J. 17, 1167-1182.
Kwon, K. C., Chan, H. T., Leon, I. R., Williams-Carrier, R., Barkan, A. and Daniell, H. (2016) Codon optimization to enhance expression yields insights into chloroplast translation. Plant Physiol. 172, 62-77.
Kwon, K. C., Sherman, A., Chang, W. J., Kamesh, A., Biswas, M., Herzog, R. W. and Daniell, H. (2018) Expression and assembly of largest foreign protein in chloroplasts: oral delivery of human FVIII made in lettuce chloroplasts robustly suppresses inhibitor formation in haemophilia A mice. Plant Biotechnol. J. 16, 1148-1160.
Lee, S. B., Li, B., Jin, S. and Daniell, H. (2011) Expression and characterization of antimicrobial peptides Retrocyclin-101 and Protegrin-1 in chloroplasts to control viral and bacterial infections. Plant Biotechnol. J. 9, 100-115.
Liu, Y., Kamesh, A. C., Xiao, Y., Sun, V., Hayes, M., Daniell, H. and Koo, H. (2016) Topical delivery of low-cost protein drug candidates made in chloroplasts for biofilm disruption and uptake by oral epithelial cells. Biomaterials, 105, 156-166.
Ma, J. K. C., Hikmat, B. Y., Wycoff, K., Vine, N. D., Chargelegue, D., Yu, L., Hein, M. B. et al. (1998) Characterization of a recombinant plant monoclonal secretory antibody and preventive immunotherapy in humans. Nat. Med. 4, 601-606.
Mural, S., Igarashi, A., Ito, K., Kanamori, S. and Otani, K. (1975) On the inhibitory effect of dental plaque in man by use of dextranase-containing chewing-gum (author's transl). Nihon Shishubyo Gakkai kaishi, 17, 74-79.
Otsuka, R., Imai, S., Murata, T., Nomura, Y., Okamoto, M., Tsumori, H., Kakuta, E. et al. (2015) Application of chimeric glucanase comprising mutanase and dextranase for prevention of dental biofilm formation. Microbiol. Immunol. 59, 28-36.
Park, J., Yan, G., Kwon, K. C., Liu, M., Gonnella, P. A., Yang, S. and Daniell, H. (2020) Oral delivery of novel human IGF-1 bioencapsulated in lettuce cells promotes musculoskeletal cell proliferation, differentiation and diabetic fracture healing. Biomaterials, 233, 119591. 10.1016/j.biomaterials.2019.119591.
Patel, J. and Sampson, V. (2020) The role of oral bacteria in COVID-19. Lancet Microbe, 1, e105. 10.1016/S2666-5247(20)30057-4
Peres, M. A., Macpherson, L. M. D., Weyant, R. J., Daly, B., Venturelli, R., Mathur, M. R., Listl, S. et al. (2019) Oral diseases: a global public health challenge. Lancet, 394, 249-260.
Pogue, G. P., Vojdani, F., Palmer, K. E., Hiatt, E., Hume, S., Phelps, J., Long, L. et al. (2010) Production of pharmaceutical-grade recombinant aprotinin and a monoclonal antibody product using plant-based transient expression systems. Plant Biotechnol. J. 8, 638-654.
Prabhawathi, V., Boobalan, T., Sivakumar, P. M. and Doble, M. (2014) Antibiofilm properties of interfacially active lipase immobilized porous polycaprolactam prepared by LB technique. PLoS One, 9, e96152. 10.1371/journal.pone.0096152.
Purushe, S., Prakash, D., Nawani, N. N., Dhakephalkar, P. and Kapadnis, B. (2012) Biocatalytic potential of an alkalophilic and thermophilic dextranase as a remedial measure for dextran removal during sugar manufacture. Bioresour. Technol. 115, 2-7.
Ramessar, K., Rademacher, T., Sack, M., Stadlmann, J., Platis, D., Stiegler, G., Labrou, N. et al. (2008) Cost-effective production of a vaginal protein microbicide to prevent HIV transmission. Proc. Natl Acad. Sci. USA, 105, 3727-3732.
Rella, A., Farnoud, A. M. and Del Poeta, M. (2016) Plasma membrane lipids and their role in fungal virulence. Prog. Lipid Res., 61, 63-72.
Ren, Z., Kim, D., Paula, A. J., Hwang, G., Liu, Y., Li, J., Daniell, H. et al. (2019) Dual-targeting approach degrades biofilm matrix and enhances bacterial killing. J. Dent. Res. 98, 322-330.
Ruhlman, T., Verma, D., Samson, N. and Daniell, H. (2010) The role of heterologous chloroplast sequence elements in transgene integration and expression. Plant Physiol. 152, 2088-2104.
Schindelin, J., Arganda-Carreras, I., Frise, E., Kaynig, V., Longair, M., Pietzsch, T., Preibisch, S. et al. (2012) Fiji: an open-source platform for biological-image analysis. Nat. Methods, 9, 676-682.
Seghal Kiran, G., Nishanth Lipton, A., Kennedy, J., Dobson, A. D. and Selvin, J. (2014) A halotolerant thermostable lipase from the marine bacterium Oceanobacillus sp. PUMB02 with an ability to disrupt bacterial biofilms. Bioengineered, 5, 305-318.
Somogyi, M. (1945) A new reagent for the determination of sugars. J. Biol. Chem. 160, 61-68.
Su, J., Zhu, L., Sherman, A., Wang, X., Lin, S., Kamesh, A., Norikane, J. H. et al. (2015) Low cost industrial production of coagulation factor IX bioencapsulated in lettuce cells for oral tolerance induction in hemophilia B. Biomaterials, 70, 84-93.
Takaki, P., Vieira, M. and Bommarito, S. (2014) Maximum bite force analysis in different age groups. Int Arch Otorhinolaryngol, 18, 272-276.
Vamvaka, E., Arcalis, E., Ramessar, K., Evans, A., O'Keefe, B. R., Shattock, R. J., Medina, V. et al. (2016b) Rice endosperm is cost-effective for the production of recombinant griffithsin with potent activity against HIV. Plant Biotechnol. J. 14, 1427-1437.
Vamvaka, E., Evans, A., Ramessar, K., Krumpe, L. R. H., Shattock, R. J., O'Keefe, B. R., Christou, P. and et al. (2016a) Cyanovirin-N produced in rice endosperm offers effective pre-exposure prophylaxis against HIV-1 BaL infection in vitro. Plant Cell Rep. 35, 1309-1319.
van Doremalen, N., Bushmaker, T., Morris, D. H., Holbrook, M. G., Gamble, A., Williamson, B. N., Tamin, A. et al. (2020) Aerosol and surface stability of SARS-CoV-2 as compared with SARS-CoV-1. N. Engl. J. Med. 382, 1564-1567.
Varga, S., Spalj, S., Lapter Varga, M., Anic Milosevic, S., Mestrovic, S. and Slaj, M. (2011) Maximum voluntary molar bite force in subjects with normal occlusion. Eur. J. Orthodont. 33, 427-433.
Verma, D., Samson, N. P., Koya, V. and Daniell, H. (2008) A protocol for expression of foreign genes in chloroplasts. Nat. Protoc. 3, 739-758.
Vos, T., Abajobir, A. A., Abate, K. H., Abbafati, C., Abbas, K. M., Abd-Allah, F., Abdulkader, R. S. et al. (2017) Global, regional, and national incidence, prevalence, and years lived with disability for 328 diseases and injuries for 195 countries, 1990-2016: a systematic analysis for the Global Burden of Disease Study 2016. Lancet, 390, 1211-1259.
Wessel, S. W., van der Mei, H. C., Maitra, A., Dodds, M. W. and Busscher, H. J. (2016) Potential benefits of chewing gum for the delivery of oral therapeutics and its possible role in oral healthcare. Expert Opin. Drug Deliv. 13, 1421-1431.
WHO. (2020) Transmission of SARS-CoV-2: implications for infection prevention precautions: scientific brief, 9 Jul. 2020 (No. WHO/2019-nCoV/Sci_Brief/Transmission_modes/2020.3). World Health Organization.
Xu, R., Cui, B., Duan, X., Zhang, P., Zhou, X. and Yuan, Q. (2020) Saliva: potential diagnostic value and transmission of 2019-nCoV. Int. J. Oral Sci. 12, 1-6.
Yong, E. D. (2010) Human jaws are surprisingly strong and efficient. Available on the world wide web at nationalgeographic.com/science/phenomena/2010/06/22/who-are-you-calling-weak-human-jaws-are-surprisingly-strong-and-efficient
Yusibov, V., Streatfield, S. J. and Kushnir, N. (2011) Clinical development of plant-produced recombinant pharmaceuticals: vaccines, antibodies and beyond. Hum Vaccin. 7, 313-321.
Zeitlin, L., Olmsted, S. S., Moench, T. R., Co, M. S., Martinell, B. J., Paradkar, V. M., Russell, D. R. et al. (1998) A humanized monoclonal antibody produced in transgenic plants for immunoprotection of the vagina against genital herpes. Nat. Biotechnol. 16, 1361-1364.
Singh R, Ren Z, Shi Y, et al. Affordable oral health care: dental biofilm disruption using chloroplast made enzymes with chewing gum delivery. Plant Biotechnol J. 2021; 19(10):2113-2125. doi:10.1111/pbi.13643. Additional biofilm degrading enzyme encoding sequences useful in the practice of the invention, include without limitation,

Complete coding DNA sequence Smdex gene dextranase from
Streptococcus mutans strain ATCC 25175
(SEQ ID NO: 34)
ATGGAACAGTCAAATAGGCAAACGGCTGAACCAGCTATTAGGTCAAATGAA
ACGGTGGATTCGGCCATTAACTCTTTTCAAGAGACAGACCTTAAGGTGCAAGAGAA
GGAGGATGCTGCGGCTGCAGTACAGACAGAACCGGCGTCAATAGATTCTAATGAAC
AGGGACAATCGGTCTCTGCAAATACTAACACACAATCTCAAGCGAAGAAACTTTCT
AACAATTCCCATCAGGAGCCAATGCAAATGGCATCTGCCGCCAATAAAGAAAGGGT
TGTGCTAGAAACTGCACAGAATCAAAAGAATGGCAACATGATAAATCTGACAACAG
ATAAAGCAGTCTACCAGGCGGGAGAGGCTGTTCATTTGAACCTTACTTTAAACAATA
CAACATCTTTAGCCCAAAATATTACAGCTACTGCTGAGGTTTATTCCCTTGAAAATA
AATTAAAGACACTTCAGTATACGAAGTATCTTCTGCCTAATGAAAGTTATACAACTC
AAAAAGGTGAATTCGTTATTCCTGCAAACTCCTTAGCTAATAATCGCGGTTATCTTTT
GAAGGTTAACATATCAGATAGCCAAAATAATATTTTAGAGCAGGGCAATCGGGCTA
TTGCGGTTGAGGATGACTGGCGTACCTTTCCGCGTTATGCTGCTATTGGAGGATCTC
AAAAAGACAATAACAGTGTCTTGACTAAGAACTTACCAGATTATTATCGCGAATTAG
AGCAGATGAAAAATATGAACATTAATTCCTATTTCTTCTATGATGTTTATAAGTCTGC
TACAAATCCTTTCCCTAATGTTCCTAAGTTTGATCAGTCTTGGAATTGGTGGAGCCAT
TCGCAGGTTGAAACAGATGCTGTTAAAGCCTTGGTCAATCGTGTCCATCAAACTGGC
GCTGTTGCCATGCTCTATAATATGATTTTAGCACAGAATGCTAATGAAACGGCTGTT
TTACCAGATACTGAGTACATCTATAATTATGAGACTGGTGGTTATGGTCAAAATGGT
CAGGTCATGACTTACTCTATTGATGATAAGCCGCTGCAATATTATTACAATCCTTTGA
GTAAAAGTTGGCAAAATTATATTTCTAATGCAATGGCTCAAGCTATGAAAAATGGCG
GTTTTGATGGCTGGCAGGGAGATACAATTGGAGATAATCGTGTTCTTTCCCATAACC
AAAAGGACAGTCGAGATATTGCTCATTCCTTTATGTTATCTGATGTCTATGCTGAATT
TCTCAATAAAATGAAGGAAAAACTGCCTCAGTATTATTTAACACTCAATGATGTTAA
TGGTGAAAATATCAGCAAACTCGCCAACAGCAAACAAGATGTGATTTACAATGAAT
TATGGCCTTTTGGAACTTCAGCTTTGGGGAACCGTCCCCAAGAAAGTTATGGTGACT
TGAAAGCTCGTGTTGATCAAGTTCGCCAAGCGACAGGGAAATCTTTGATTGTCGGAG
CTTATATGGAAGAGCCTAAATTTGATGATAATAGGATTCCTCTCAATGGTGCAGCGC
GTGACGTTTTAGCTTCAGCAACTTACCAAACAGATGCGGTTCTGCTGACAACTGCGG
CCATTGCGGCAGCAGGAGGATATCACATGTCTCTGGCTGCTCTGGCTAATCCTAATG
ATGGGGGTGGTGTCGGTGTCTTAGAAACAGCTTATTATCCAACACAAAGCCTCAAGG
TTTCGAAAGAGCTCAATCGTAAAAACTATCATTACCAACAATTTATTACGGCTTATG
AAAATCTTTTGCGTGATAAAGTTGAAAATGATTCTGCTGAACCTCAGACTTTCACTG
CTAACGGTCGGCAGCTATCGCAAGATGCTTTGGGGATCAATGGCGATCAGGTTTGGA
CTTATGCCAAAAAGGGAAACGATTTCAGAACGATTCAATTGCTCAACCTTATGGGAA
TTACATCCGACTGGAAAAATGAAGATGGTTATGAAAATAATAAAACACCTGATGAG
CAAACCAATTTATTGGTTACTTATCCTTTGACTGGTGTGTCTATGGCAGAGGCTGATC
GAATAGCTAAACAAGTCTATCTGACGTCACCAGATGATTGGCTGCAATCTAGTATGA
TTTCTCTAGCGACTCAGGTAAAAACGAATGAGAATGGCGATCCTGTTCTTTATATTC
AAGTGCCAAGACTGACGCTTTGGGATATGATTTATATTAATGAAACCATTAAACCAG
AAACGCCTAAAGTTCCAGAACAGCCCCAACATCCTGCTAGGACACTTGAACCAGCA
ATTCCGCAAACTCCAGAAGCAGTCAACCCTCTCCCAGTAGCTAATAAGCAGGCAGT
AGATGAAAATAAAAATGAGATTGTTTCAGCCTTAACCGGTGAAGAAAATGACTTGC
AGTTGCCAACTCTTTCCAAACAATCATTGCCAATCTCCCAAGCAGAGTTACCGCAAA
CAGGAGATAACAATGAAACGCGCTCCAATCTCCTCAAAGTGATAGGTGCTGGTGCG
CTTCTAATCGGCGCTGCAGGATTATTAAGCTTGATAAAGGGTAGAAAAAATGATTGA
Complete coding translated amino acid sequence Smdex gene
dextranase from Streptococcus mutans strain ATCC 25175
(SEQ ID NO: 35)
MEQSNRQTAEPAIRSNETVDSAINSFQETDLKVQEKEDAAAAVQTEPASIDSNEQ
GQSVSANTNTQSQAKKLSNNSHQEPMQMASAANKERVVLETAQNQKNGNMINLTTDK
AVYQAGEAVHLNLTLNNTTSLAQNITATAEVYSLENKLKTLQYTKYLLPNESYTTQKGE
FVIPANSLANNRGYLLKVNISDSQNNILEQGNRAIAVEDDWRTFPRYAAIGGSQKDNNS
VLTKNLPDYYRELEQMKNMNINSYFFYDVYKSATNPFPNVPKFDQSWNWWSHSQVET
DAVKALVNRVHQTGAVAMLYNMILAQNANETAVLPDTEYIYNYETGGYGQNGQVMT
YSIDDKPLQYYYNPLSKSWQNYISNAMAQAMKNGGFDGWQGDTIGDNRVLSHNQKDS
RDIAHSFMLSDVYAEFLNKMKEKLPQYYLTLNDVNGENISKLANSKQDVIYNELWPFGT
SALGNRPQESYGDLKARVDQVRQATGKSLIVGAYMEEPKFDDNRIPLNGAARDVLASA
TYQTDAVLLTTAAIAAAGGYHMSLAALANPNDGGGVGVLETAYYPTQSLKVSKELNR
KNYHYQQFITAYENLLRDKVENDSAEPQTFTANGRQLSQDALGINGDQVWTYAKKGN
DFRTIQLLNLMGITSDWKNEDGYENNKTPDEQTNLLVTYPLTGVSMAEADRIAKQVYL
TSPDDWLQSSMISLATQVKTNENGDPVLYIQVPRLTLWDMIYINETIKPETPKVPEQPQH
PARTLEPAIPQTPEAVNPLPVANKQAVDENKNEIVSALTGEENDLQLPTLSKQSLPISQAE
LPQTGDNNETRSNLLKVIGAGALLIGAAGLLSLIKGRKND
Codon optimized Paenibacillus sp. mut gene DNA Sequence
(SEQ ID NO: 36)
ATGGCAGGTGGCCCGAATCTTACTCCAGGTAAACCAATTACTGCTAGTGGTC
AATCTCAAACCTATAGCCCTCAAAATGTAAAAGATGGCAATCAAAATACTTACTGG
GAAAGTACTAACAATGCCTTCCCTCAATGGATTCAAGTTGATTTGGGTGCAAGTACT
GGCATTGATCAAATTGTTCTTAAGTTACCAGCTAGCTGGGAAGCTCGTACTCAAACT
CTTGCTGTTCAAGGTAGTTTGAATGGTTCTACTTTCACTGATATTGTAGGTTCTGCAA
ATTATGTATTCAGTCCTTCTGTAGGTAATAACACTGTTACTATTAATTTTACCGCCAC
AAGCACCCGTTATGTTCGCTTGTACGTAACTGCGAACACTGGTTGGCCAGCTGCTCA
ACTGTCTGAATTAGAAATTTATGGTTCTGGTGACCAGACTCCTGCACCTGATACTTAT
CAAGCTGAAAGTGCTGCTTTATCTGGTGGCGCTAAAGTAAATACTGATCATGCCGGC
TACATAGGTACTGGTTTTGTTGATGGTTATTGGACTCAAGGCGCTACTACTACCTTTT
CTGTAAACGCGCCTACTGCTGGTAATTACGATGTAACTCTGAGGTATGGTAACGCAA
CCGGCAGTAATAAAACTGTATCCTTGTACGTAAATGGCGCTAAAATTCGTCAAACAA
CTTTACCAAGTCTACCTAACTGGGATTCATGGAGTAGCAAGACTGAAACTCTTAATT
TAAATGCTGGTAGCAACACCATTGCTTATAAATACGACCCTGGCGATTCTGGTAATG
TAAATCTTGATCAAATCACTGTAGAAGCATCTACTTCAACTCCTACTCCTACTCCATC
TCCTACTCCTACACCTACTCCAACTCCTACTCCTACTCCTACTCCTACACCAACACCT
ACTCCTACCCCAACCCCTACTCCTACACCTACACCTACACCTACTCCTACTCCTCCTC
CTGGTGGTAATATTGCCATAGGCAAATCTATTTCCGCATCTAGTCACACTCAAACTT
ATGTTGCTGAGAACGCAAATGATAACGATGTAAATACTTACTGGGAAGGTGGCGGT
AATCCTAGTACTTTAACTTTGGATCTTGGCGCTAATTATAATATTACTTCTATTGTTC
TAAAACTAAACCCATCCTCTATATGGGCAGCCCGTACTCAAACTATTCAAGTTTTGG
GCCATGATCAAAATACTACTACATTCAGTAATTTAGTATCTGCTAAATCTTACTCTTT
CGATCCTGCTTCTGGTAATACTGTTACCATTCCAGTTACCGCTACTGTTAAACGTTTG
CAGTTGAACATTACTTCTAATTCCGGTGCCCCTGCTGGTCAAGTAGCTGAGTTCCAA
GTTTTCGGTACTCCTGCTCCAAATCCTGATTTGACTATTACCGGTATGTCTTGGTCTC
CTTCTTCTCCAGTTGAGACAGATGCAATTACTCTGAATGCTACTGTTAAAAACAATG
GTAATGCCAGTGCAGCCGCTACCACCGTAAATTTCTACCTAAATAACGAGCTAGCTG
GTTCTGCTCCTGTAGCAGCTCTAGCGGCAGGCGCTTCTGCAACTGTTCCGCTAAATG
TAGGTGCTAAAACCGCCGCCACATACGCTGTAGGTGCTAAAGTAGATGAAAGTAAT
GCAGTAATTGAGTTAAACGAGTCTAACAATAGCTACACTAATCCTGCTTCATTGGTT
GTTGCTCCAGTTAGTAGTTCTGATTTAGTTGGCACTGTTTCTTGGACTCCAAGCACTC
CTATTGCAAACAATGCTGTTTCTTTTAACGTAAATCTTAAAAATCAAGGCACTATTG
CTTCTGCCGGTGGTTCTCACGGTGTTACTGTAGTTCTTAAAAATGCTTCCGGTTCTAC
CGTTCAAACTTTCAGTGGTTCTTACACCGGTAGTCTTGCTCCGGGAGCTTCCGTAAAT
ATTACCCTTCCTGGTACCTGGACTGCTGCTGCTGGTAGCTATACTGTAACTGCAACC
GTTGCGGCAGACGCTAACGAACTTCCTATCAAGCAAGCCAACAATGCAAACACAGC
AAGTCTAACCGTATATTCTGCTCGTGGTGCAAGCATGCCATACAGTCGTTACGATAC
CGAGGATGCCACCCTTGGTGGTGGCGCTACTCTAAAATCCGCTCCGACATTCGATCA
AGCGCTTACTGCATCTGAAGCCACCGGTCAATTGTACGCTGCGTTACCATCTAACGG
CTCTTATCTTCAATGGACCGTACGTCAAGGTCAGGGTGGTGCAGGCGTTACTATGAG
ATTTACTATGCCAGATTCTGCTGACGGCATGGGCTTAAACGGTAGTTTAGATGTTTA
CGTAAACGGTACAAAAGTAAAAACCGTATCTCTAACCAGTTACTATAGCTGGCAGT
ATTTCTCTGGTGATATGCCAGGAGACGCTCCAAGCGCTGGTCGTCCTTTATTCCGTTT
TGATGAAGTTCATTGGAAATTAGATACTCCTTTGAAACCAGGAGATACTATTCGCAT
ACAAAAGAACAACGGTGATAGCCTAGAATACGGTGTAGACTTTATTGAAATTGAAC
CAGTTCCTGCTGCTATCTCTCGTCCGGCTAACTCTGTTTCCGTAACTGATTACGGTGC
TGTTCCTAACGATGGACAGGACGATCTTACCGCTTTTAAAGCAGCCGTAAACGCAGC
TGTAGCATCCGATAAAATCTTGTATATTCCAGAAGGCACTTTCCACTTGGGTAACAT
GTGGGAGATTGGTTCCGTAAGTAACATGATCGATCACATTACTATTACTGGAGCTGG
TATTTGGTACACTAACATCCAGTTTACCAACGCCAATCCTGCTTCCGGTGGCATCTCT
CTACGTATTACTGGTAAACTTGATTTCAGCAACGTTTACTTGAACTCTAATTTGCGTT
CTCGTTATGGTCAAAATGCCGTTTATAAAGGTTTTATGGATAACTTCGGTACCAATTC
CGTAATTCGTGACGTATGGGTAGAACACTTCGAATGTGGTTTCTGGGTAGGTGATTA
CGGTCATACTCCTGCTATTCGCGCAAGCGGTCTGTTAATTGAAAACAGCCGAATCCG
TAACAACCTAGCTGATGGTGTAAACTTCGCCCAAGGTACCAGCAATTCTACCGTACG
CAACAGCAGCTTACGTAACAACGGTGATGACGCCCTTGCTGTATGGACTAGTAATAC
TAACGGTGCTCCAGAAGGCGTAAACAATACCTTCTCTTACAACACCATCGAAAACA
ACTGGCGCGCTGGAGGTATTGCCTTCTTCGGAGGAAGCGGACATAAGGCCGATCAC
AACTACATAGTAGATTGTGTAGGTGGTTCTGGTATCCGTATGAATACCGTTTTCCCA
GGATATCACTTCCAGAACAATACCGGTATTGTTTTCTCTGACACTACCATAGTAAAC
TGCGGTACTAGCAAAGATCTATACAACGGTGAACGCGGTGCTATCGATTTGGAAGC
ATCTAACGACGCCATCAGAAACGTTACTTTTACCAACATCGATATTATCAACTCTCA
GCGCGATGCTATCCAGTTCGGTTATGGTGGTGGTTTCACCAATATCGTTTTCAACAA
CATCAACATTAACGGAACCGGTCTTGATGGTGTAACCACCTCTCGTTTCTCTGGACC
TCATTTAGGCGCGGCGATCTTCACCTATACCGGTAACGGTAGTGCTACTTTCAACAA
TTTACGCACCAGCAATATCGCTTATCCAAATTTATATTATATCCAGAGCGGTTTCAAT
TTAATCATCAATAATTGA
Codon optimized Paenibacillus sp. mut gene amino acid Sequence
(SEQ ID NO: 37)
MAGGPNLTPGKPITASGQSQTYSPQNVKDGNQNTYWESTNNAFPQWIQVDLGA
STGIDQIVLKLPASWEARTQTLAVQGSLNGSTFTDIVGSANYVFSPSVGNNTVTINFTATS
TRYVRLYVTANTGWPAAQLSELEIYGSGDQTPAPDTYQAESAALSGGAKVNTDHAGYI
GTGFVDGYWTQGATTTFSVNAPTAGNYDVTLRYGNATGSNKTVSLYVNGAKIRQTTLP
SLPNWDSWSSKTETLNLNAGSNTIAYKYDPGDSGNVNLDQITVEASTSTPTPTPSPTPTP
TPTPTPTPTPTPTPTPTPTPTPTPTPTPTPTPPPGGNIAIGKSISASSHTQTYVAENANDNDV
NTYWEGGGNPSTLTLDLGANYNITSIVLKLNPSSIWAARTQTIQVLGHDQNTTTFSNLVS
AKSYSFDPASGNTVTIPVTATVKRLQLNITSNSGAPAGQVAEFQVFGTPAPNPDLTITGM
SWSPSSPVETDAITLNATVKNNGNASAAATTVNFYLNNELAGSAPVAALAAGASATVP
LNVGAKTAATYAVGAKVDESNAVIELNESNNSYTNPASLVVAPVSSSDLVGTVSWTPS
TPIANNAVSFNVNLKNQGTIASAGGSHGVTVVLKNASGSTVQTFSGSYTGSLAPGASVN
ITLPGTWTAAAGSYTVTATVAADANELPIKQANNANTASLTVYSARGASMPYSRYDTE
DATLGGGATLKSAPTFDQALTASEATGQLYAALPSNGSYLQWTVRQGQGGAGVTMRF
TMPDSADGMGLNGSLDVYVNGTKVKTVSLTSYYSWQYFSGDMPGDAPSAGRPLFRFD
EVHWKLDTPLKPGDTIRIQKNNGDSLEYGVDFIEIEPVPAAISRPANSVSVTDYGAVPND
GQDDLTAFKAAVNAAVASDKILYIPEGTFHLGNMWEIGSVSNMIDHITITGAGIWYTNIQ
FTNANPASGGISLRITGKLDFSNVYLNSNLRSRYGQNAVYKGFMDNFGTNSVIRDVWVE
HFECGFWVGDYGHTPAIRASGLLIENSRIRNNLADGVNFAQGTSNSTVRNSSLRNNGDD
ALAVWTSNTNGAPEGVNNTFSYNTIENNWRAGGIAFFGGSGHKADHNYIVDCVGGSGI
RMNTVFPGYHFQNNTGIVFSDTTIVNCGTSKDLYNGERGAIDLEASNDAIRNVTFTNIDII
NSQRDAIQFGYGGGFTNIVFNNININGTGLDGVTTSRFSGPHLGAAIFTYTGNGSATFNN
LRTSNIAYPNLYYIQSGFNLIINN

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1-23. (canceled)

24. A biofilm degrading composition harboring a nucleic acid encoding mutanase of SEQ ID NO:1 in a pharmaceutically acceptable carrier.

25. The biofilm degrading composition of claim 24, produced in a plant plastid and comprising a plant remnant, wherein the plant remnant is freeze dried.

26. The composition of claim 24, further comprising dextranase and lipase.

27. The composition of claim 26, wherein said mutanase, dextranase and lipase are produced in a plant plastid and are present in a plant remnant.

28. The composition of claim 25 further comprising at least one biofilm degrading enzyme selected from dextranase, lipase, glucoamylase, glucanase, deoxyribonuclease I, DNAase, dispersin B, glycoside hydrolases, and the enzymes listed in Table 2.

29. The composition claim 28, wherein the at least one biofilm degrading enzyme has a sequence selected from SEQ ID NOS: 2, 12, 14, 16, 18, 20, 24, and 26.

30. The composition of claim 26, further comprising an AMP.

31. The composition as claimed in claim 26 further comprising an antimicrobial/antibiotic.

32. The composition as claimed in claim 28, further comprising fluoride and, or CHX.

33. The composition of claim 26, wherein said carrier is chewing gum.

34. The compositions of claim 26, wherein said carrier is selected from a lozenge, a candy, and a dissolvable strip.

35. The composition of claim 26, wherein said carrier is an oral rinse.

36. A method of degrading and/or removing biofilm harboring undesirable microorganisms, comprising contacting a surface harboring said biofilm with an effective amount of the composition of claim 26, said composition having an antimicrobial effect, and reducing or eliminating said biofilm.

37. The method of claim 36, wherein said biofilm is present in the mouth and said contacting is via chewing gum comprising said enzymes.

38. The method of claim 36, wherein said biofilm is present on a medical implant.

39. The composition of claim 26, wherein the plant remnants are from a tobacco or a lettuce plant.

40. The composition of claim 26, wherein the dextranase and mutanase are present in a 5:1 ratio.

43. A method of for inhibiting biofilm deposition on a surface, comprising pre-treating said surface with the composition of claim 26, said composition having a antimicrobial effect, and inhibiting formation of said biofilm on said surface.


SEQUENCE LISTING (38 sequences)

SEQ ID NO: 1 - DNA, 3801 bp, Artificial Sequence
  codon optimized mutanase
gcaggtggcccgaatcttactccaggtaaaccaattactgctagtggtcaatctcaaacctatagccctcaaaatgtaaaagatggcaatcaaaatacttactgggaaagtactaacaatgccttccctcaatggattcaagttgatttgggtgcaagtactggcattgatcaaattgttcttaagttaccagctagctgggaagctcgtactcaaactcttgctgttcaaggtagtttgaatggttctactttcactgatattgtaggttctgcaaattatgtattcagtccttctgtaggtaataacactgttactattaattttaccgccacaagcacccgttatgttcgcttgtacgtaactgcgaacactggttggccagctgctcaactgtctgaatttgaaatttatggttctggtgaccagactcctgcacctgatacttatcaagctgaaagtgctgctttatctggtggcgctaaagtaaatactgatcatgccggctacataggtactggttttgttgatggttattggactcaaggcgctactactaccttttctgtaaacgcgcctactgctggtaattacgatgtaactctgaggtatggtaacgcaaccggcagtaataaaactgtatccttgtacgtaaatggcgctaaaattcgtcaaacaactttaccaagtctacctaactgggattcatggagtagcaagactgaaactcttaatttaaatgctggtagcaacaccattgcttataaatacgaccctggcgattctggtaatgtaaatcttgatcaaatcactgtagaagcatctacttcaactcctactcctactccatctcctactcctacacctactccaactcctactcctactcctactcctacaccaacacctactcctaccccaacccctactcctacacctacacctacacctactcctactcctcctcctggtggtaatattgccataggcaaatctatttccgcatctagtcacactcaaacttatgttgctgagaacgcaaatgataacgatgtaaatacttactgggaaggtggcggtaatcctagtactttaactttggatcttggcgctaattataatattacttctattgttctaaaactaaacccatcctctatatgggcagcccgtactcaaactattcaagttttgggccatgatcaaaatactactacattcagtaatttagtatctgctaaatcttactctttcgatcctgcttctggtaatactgttaccattccagttaccgctactgttaaacgtttgcagttgaacattacttctaattccggtgcccctgctggtcaagtagctgagttccaagttttcggtactcctgctccaaatcctgatttgactattaccggtatgtcttggtctccttcttctccagttgagacagatgcaattactctgaatgctactgttaaaaacaatggtaatgccagtgcagccgctaccaccgtaaatttctacctaaataacgagctagctggttctgctcctgtagcagctctagcggcaggcgcttctgcaactgttccgctaaatgtaggtgctaaaaccgccgccacatacgctgtaggtgctaaagtagatgaaagtaatgcagtaattgagttaaacgagtctaacaatagctacactaatcctgcttcattggttgttgctccagttagtagttctgatttagttggcactgtttcttggactccaagcactcctattgcaaacaatgctgtttcttttaacgtaaatcttaaaaatcaaggcactattgcttctgccggtggttctcacggtgttactgtagttcttaaaaatgcttccggttctaccgttcaaactttcagtggttcttacaccggtagtcttgctccgggagcttccgtaaatattacccttcctggtacctggactgctgctgctggtagctatactgtaactgcaaccgttgcggcagacgctaacgaacttcctatcaagcaagccaacaatgcaaacacagcaagtctaaccgtatattctgctcgtggtgcaagcatgccatacagtcgttacgataccgaggatgccacccttggtggtggcgctactctaaaatccgctccgacattcgatcaagcgcttactgcatctgaagccaccggtcaattgtacgctgcgttaccatctaacggctcttatcttcaatggaccgtacgtcaaggtcagggtggtgcaggcgttactatgagatttactatgccagattctgctgacggcatgggcttaaacggtagtttagatgtttacgtaaacggtacaaaagtaaaaaccgtatctctaaccagttactatagctggcagtatttctctggtgatatgccaggagacgctccaagcgctggtcgtcctttattccgttttgatgaagttcattggaaattagatactcctttgaaaccaggagatactattcgcatacaaaagaacaacggtgatagcctagaatacggtgtagactttattgaaattgaaccagttcctgctgctatctctcgtccggctaactctgtttccgtaactgattacggtgctgttcctaacgatggacaggacgatcttaccgcttttaaagcagccgtaaacgcagctgtagcatccgataaaatcttgtatattccagaaggcactttccacttgggtaacatgtgggagattggttccgtaagtaacatgatcgatcacattactattactggagctggtatttggtacactaacatccagtttaccaacgccaatcctgcttccggtggcatctctctacgtattactggtaaacttgatttcagcaacgtttacttgaactctaatttgcgttctcgttatggtcaaaatgccgtttataaaggttttatggataacttcggtaccaattccgtaattcgtgacgtatgggtagaacacttcgaatgtggtttctgggtaggtgattacggtcatactcctgctattcgcgcaagcggtctgttaattgaaaacagccgaatccgtaacaacctagctgatggtgtaaacttcgcccaaggtaccagcaattctaccgtacgcaacagcagcttacgtaacaacggtgatgacgcccttgctgtatggactagtaatactaacggtgctccagaaggcgtaaacaataccttctcttacaacaccatcgaaaacaactggcgcgctggaggtattgccttcttcggaggaagcggacataaggccgatcacaactacatagtagattgtgtaggtggttctggtatccgtatgaataccgttttcccaggatatcacttccagaacaataccggtattgttttctctgacactaccatagtaaactgcggtactagcaaagatctatacaacggtgaacgcggtgctatcgatttggaagcatctaacgacgccatcagaaacgttacttttaccaacatcgatattatcaactctcagcgcgatgctatccagttcggttatggtggtggtttcaccaatatcgttttcaacaacatcaacattaacggaaccggtcttgatggtgtaaccacctctcgtttctctggacctcatttaggcgcggcgatcttcacctataccggtaacggtagtgctactttcaacaatttacgcaccagcaatatcgcttatccaaatttatattatatccagagcggtttcaatttaatcatcaataatcatcatcaccatcaccactaa

SEQ ID NO: 2 - DNA, 2553 bp, Artificial Sequence
  codon optimized dextranase
atggaacagtcaaataggcaaacggctgaaccagctattaggtcaaatgaaacggtggattcggccattaactcttttcaagagacagaccttaaggtgcaagagaaagaggatgctgcggctgcagtacagacagaatcggcgtcaatagattctaatgaacaggaacaatcggtctctgcaaatactaacacacaacctcaagcgaagaaactttctaacaattcccatcaggagccaatgcaaatggtatctgccgccaataaagaaagggctgtgctagaaactgcacagaaccaaaagaatggcaacatgataaatctgacaacagataaagcagtctatcaggcgggagaggctgttcatttgaatcttactttaaacaatacaacatctttagcccaaaatattacagctactgttgaggtttattcccttgaaaataaattaaagacacttcagtatacgaagtatcttctgcctaatgaaagttatacaactcaaaaaggtgaattcgttattcctgcaaactccttagctaataatcgcggttatcttttgaaggttaacatatcagatagccaaaataatattttagagcagggcaatcgggctattgcggttgaggatgactggcgtacctttccgcgttatgctgctattggaggttctcaaaaagacaataacagtgtcttgactaagaacttaccagattattatcgcgaattagagcagatgaaaaatatgaacattaattcctatttcttctatgatgtttataagtctgctacaaatcctttccctaatgttcctaagtttgatcagtcttggaattggtggagccattcgcaggttgaaacagatgctgttaaagccttggtcaatcgtgtccatcaaactggcgctgttgccatgctctataatatgattttagcacagaatgctaatgaaacggctgttttaccagatactgagtacatctataattatgagactggtggttatggtcaaaatggtcaggtcatgacttactctattgatgataagccactgcaatattattacaatcctttgagtaaaagttggcaaaattatatttctaatgccatggctcaagctatgaaaactggcggttttgatggctggcagggagatacaattggagataatcgtgttctttcccataatcaaaaggacagtcgagatattgctcattcctttatgttatctgatgtctatgctgaatttctcaataaaatgaaggaaaaactgcctcagtattatttaacactcaatgatgttaatggtgaaaatatcagcaaactcgccaacagcaaacaagatgtgatttacaatgaattatggccttttggaacttcagctttggggaaccgtccccaagaaagttatggtgacttgaaagctcgtgttgatcaagttcgccaagcgacagggaaatctttgattgtcggagcttatatggaagagcctaaatttgatgataataggattcctctcaatggtgcagcgcgtgacgttttagcttcagcaacttaccaaacagatgcggttctgctgacaactgcggccattgcggcagcaggaggatatcacatgtctctggctgctctggctaatcctaatgatgggggtggtgtcggtgtcttagaaacagcttattatccaacacaaagcctcaaggtttcgaaagagctcaatcgtaaaaactatcattaccaacaatttattacggcttatgaaaatcttttgcgtgataaagttgaaaatgattctgctgaacctcagactttcactgctaacggtcggcagctatcgcaagatgctttggggatcaatggcgatcaggtttggacttatgccaaaaagggaaacgatttcagaacgattcaattgctcaaccttatgggaattacatctgactggaaaaatgaagatggttatgaaaataataaaacacctgatgagcaaaccaatttattggttacttatcctttgactggtgtatctatggcagaggctgatcgaatagctaaacaagtctatctgacgtcaccagatgattggctgcaatctagtatgatttctctaacgactcaggtaaaaacgaatgagaatggcgatcctgttctttatattcaagtgccaagactgacgctttgggatatgatttatattaatgaaaccattaaaccagaaacgcctaaagttccagaacagccccaacatcctgctaggacacttgaaccagcaattccgcaaactccagaagcagtcagccctctcccagtagctaataagcaggcagaagatggaaataaaaatgagcttgtttcagctttaaccggtgaagaaaatgacttgcagctgccaactctttccaaacgatcattgtcaatctcccaagcagagttaccgcaaacaggagataacaatgaaacgcgctccaatctcctcaaagtgataggtgctggtgcgcttctaatcggcgctgcaggattattaagcttgataaagggtagaaaaaatgattga

SEQ ID NO: 3 - protein, 16 aa, Artificial Sequence
  CSPC16
TFFRLFNRSFTQALGK

SEQ ID NO: 4 - protein, 16 aa, Artificial Sequence
  G2
  MOD_RES (16)..(16): AMIDATION
KNLRIIRKGIHIIKKY

SEQ ID NO: 5 - protein, 35 aa, Artificial Sequence
  C16G2
  MOD_RES (35)..(35): AMIDATION
TFFRLFNRSFTQALGKGGGKNLRIIRKGIHIIKKY

SEQ ID NO: 6 - protein, 8 aa, Artificial Sequence
  CSPM8
TFFRLFNR

SEQ ID NO: 7 - protein, 27 aa, Artificial Sequence
  M8G2
  MOD_RES (27)..(27): AMIDATION
TFFRLFNRGGGKNLRIIRKGIHIIKKY

SEQ ID NO: 8 - protein, 11 aa, Artificial Sequence
  S6L3-33
FKKFWKWFRRF

SEQ ID NO: 9 - protein, 31 aa, Artificial Sequence
  C16-33
TRRRLFNRSFTQALGKSGGGFKKFWKWFRRF

SEQ ID NO: 10 - protein, 23 aa, Artificial Sequence
  M8-33
TFFRLFNRSGGGFKKFWKWFRRF

SEQ ID NO: 11 - protein, 5 aa, Artificial Sequence
  elastin-like biopolymer
GVGVP

SEQ ID NO: 12 - protein, 1146 aa, Paenibacillus humicus
MRIRTKYMNWMLVLVLIAAGFFQAAGPIAPATAAGGANLTLGKTVTASGQSQTYSPDNVKDSNQGTYWESTNNAFPQWIQVDLGASTSIDQIVLKLPSGWETRTQTLSIQGSANGSTFTNIVGSAGYTFNPSVAGNSVTINFSAASARYVRLNFTANTGWPAGQLSELEIYGATAPTPTPTPTPTPTPTPTPTPTPTVTPAPSATPTPTPPAGSNIAVGKSITASSSTQTYVAANANDNNTSTYWEGGSNPSTLTLDFGSNQSITSVVLKLNPASEWGTRTQTIQVLGADQNAGSFSNLVSAQSYTFNPATGNTVTIPVSATVKRLQLNITANSGAPAGQIAEFQVFGTPAPNPDLTITGMSWTPSSPVESGDITLNAVVKNIGTAAAGATTVNFYLNNELAGTAPVGALAAGASANVSINAGAKAAATYAVSAKVDESNAVIEQNEGNNSYSNPTNLVVAPVSSSDLVAVTSWSPGTPSQGAAVAFTVALKNQGTLASAGGAHPVTVVLKNAAGATLQTFTGTYTGSLAAGASANISVGSWTAASGTYTVSTTVAADGNEIPAKQSNNTSSASLTVYSARGASMPYSRYDTEDAVLGGGAVLRTAPTFDQSLIASEASGQKYAALPSNGSSLQWTVRQGQGGAGVTMRFTMPDTSDGMGQNGSLDVYVNGTKAKTVSLTSYYSWQYFSGDMPADAPGGGRPLFRFDEVHFKLDTALKPGDTIRVQKGGDSLEYGVDFIEIEPIPAAVARPANSVSVTEYGAVANDGKDDLAAFKAAVTAAVAAGKSLYIPEGTFHLSSMWEIGSATSMIDNFTVTGAGIWYTNIQFTNPNASGGGISLRIKGKLDFSNIYMNSNLRSRYGQNAVYKGFMDNFGTNSIIHDVWVEHFECGMWVGDYAHTPAIYASGLVVENSRIRNNLADGINFSQGTSNSTVRNSSIRNNGDDGLAVWTSNTNGAPAGVNNTFSYNTIENNWRAAAIAFFGGSGHKADHNYIIDCVGGSGIRMNTVFPGYHFQNNTGITFSDTTIINSGTSQDLYNGERGAIDLEASNDAIKNVTFTNIDIINAQRDGVQIGYGGGFENIVFNNITIDGTGRDGISTSRFSGPHLGAAIYTYTGNGSATFNNLVTRNIAYAGGNYIQSGFNLTIK

SEQ ID NO: 13 - DNA, 3640 bp, Paenibacillus humicus
aaaggaggatcgccaaccaatcatcccagcaaagaaggtgatggcagcccaagaattgaaagcgctttgaatttggaatatacggatttggccgacctgctgattcagtcgtattcaagcgattatgccgcgaaccaatcgaacccgaggaggactataatgcgtatccgcactaaatatatgaactggatgttggtgctcgtcctgatcgccgccggcttcttccaggctgccggccccatcgctcccgccaccgctgcaggaggcgcgaatctgacgctcggcaaaaccgtcaccgccagcggccagtcgcagacgtacagccccgacaatgtcaaggacagcaatcagggaacttactgggaaagcacgaacaacgccttcccgcagtggatccaagtcgaccttggcgccagcacgagcatcgaccagatcgtgctcaagcttccgtccggatgggagactcgtacgcaaacgctctcgatacagggcagcgcgaacggctcgacgttcacgaacatcgtcggatcggccgggtatacattcaatccatccgtcgccggcaacagcgtcacgatcaacttcagcgctgccagcgcccgctacgtccgcctgaatttcacggccaatacgggctggccagcaggccagctgtcggagcttgagatctacggagcgacggcgccaacgcctactcccacgcctactccaacaccaacgccaacgccaacaccaacgccaacccctacagtaacccctgcgccttcggccacgccgactccgactcctccggcaggcagcaacatcgccgtagggaaatcgattacagcctcttccagcacgcagacctacgtagctgcaaatgcaaatgacaacaatacatccacctattgggagggaggaagcaacccgagcacgctgactctcgatttcggttccaaccagagcatcacttccgtcgtcctcaagctgaatccggcttcggaatgggggactcgcacgcaaacgatccaagttcttggagcggatcagaacgccggctccttcagcaatctcgtctctgcccagtcctatacgttcaatcccgcaaccggcaatacggtgacgattccggtctccgcgacggtcaagcgcctccagctgaacattacggcgaactccggcgcccctgccggccagattgccgagttccaagtgttcggcacgccagcgcctaatccggacttgaccattaccggcatgtcctggactccgtcttctccggtcgagagcggcgacattacgctgaacgccgtcgtcaagaacatcggaactgcagctgcaggcgccacgacggtcaatttctacctgaacaacgaactcgccggcaccgctccggtaggcgcgcttgcggcaggagcttctgcaaatgtatcgatcaatgcaggcgccaaagcagccgcaacgtatgcggtaagcgccaaagtcgacgagagcaacgccgtcatcgagcagaatgaaggcaacaacagctactcgaacccgactaacctcgtcgtagcgccggtgtccagctccgacctcgtcgccgtgacgtcatggtcgccgggcacgccgtcgcagggagcggcggtcgcatttaccgtcgcgcttaaaaatcagggtacgctggcttccgccggcggagcccatcccgtaaccgtcgttctgaaaaacgctgccggagcgacgctgcaaaccttcacgggcacctacacaggttccctggcagcaggcgcatccgcgaatatcagcgtgggcagctggacggcagcgagcggcacctataccgtctcgacgacggtagccgctgacggcaatgaaattccggccaagcaaagcaacaatacgagcagcgcgagcctcacggtctactcggcgcgcggcgccagcatgccgtacagccgttacgacacggaggatgcggtgctcggcggcggagctgtcctgagaacggcgccgacgttcgatcagtcgctcatcgcttccgaagcatcgggacagaaatacgccgcacttccgtccaacggctccagcctgcagtggaccgtccgtcaaggccagggcggtgcaggcgtcacgatgcgcttcacgatgcccgacacgagcgacggcatgggccagaacggctcgctcgacgtctatgtcaacggaaccaaagccaaaacggtgtcgctgacctcttattacagctggcagtatttctccggcgacatgccggctgacgctccgggcggcggcaggccgctcttccgcttcgacgaagtccacttcaagctggatacggcgttgaagccgggagacacgatccgcgtccagaagggcggtgacagcctggagtacggcgtcgacttcatcgagatcgagccgattccggcagcggttgcccgtccggccaactcggtgtccgtcaccgaatacggcgctgtcgccaatgacggcaaggatgatctcgccgccttcaaggctgccgtgaccgcagcggtagcggccggaaaatccctctacatcccggaaggcaccttccacctgagcagcatgtgggagatcggctcggccaccagcatgatcgacaacttcacggtcacgggtgccggcatctggtatacgaacatccagttcacgaatcccaatgcatcgggcggcggcatctccctgagaatcaaaggaaagcttgatttcagcaacatctacatgaactccaacctgcgttcccgttacgggcagaacgccgtctacaaaggctttatggacaatttcggcactaattcgatcatccatgacgtctgggtcgagcatttcgaatgcggcatgtgggtcggcgactacgcccatactcctgcgatctatgcgagcgggctcgtcgtggaaaacagccgcatccgcaacaatcttgccgacggcatcaacttctcgcagggaacgagcaactcgaccgtccgcaacagcagcatccgcaacaacggcgatgacggcctcgccgtctggacgagcaacacgaacggcgctccggccggcgtgaacaacaccttctcctacaacacgatcgagaacaactggcgcgcggcggccatcgccttcttcggcggcagcggccacaaggctgaccacaactacatcatcgactgtgtcggcggctccggcatccggatgaatacggtgttcccaggctaccacttccagaacaacaccggcatcaccttctcggatacgacgatcatcaacagcggcaccagccaggatctgtacaacggcgagcgcggagcgattgatctggaagcttccaacgacgcgatcaaaaacgtcaccttcaccaacatcgacatcatcaatgcccagcgcgacggcgttcagatcggctatggcggcggcttcgagaacatcgtgttcaacaacatcacgatcgacggcaccggccgcgacgggatatcgacatcccgcttctcgggacctcatcttggcgcagccatctatacgtacacgggcaacggctcggcgacgttcaacaacctggtgacccggaacatcgcctatgcaggcggcaactacatccagagcgggttcaacctgacgatcaaataggctgcaaaaaaaaggaagctcctcggagcttccttttttt

SEQ ID NO: 14 - protein, 1261 aa, Paenibacillus curdlanolyticus
MRNKYVTWTLALTMLFSSFFLAVGPNKVVHAAGGTNLALGKNVTASGQSQTYSPNNVKDSNQSTYWESTNNAFPQWIQVDLGATTSIDQIVLKLPAGWGTRTQTLAVQGSTDGSSFTNIVGSAGYVFNPAVANNAVTINFSAASTRYVRLNVTANTAWPAAQLSEFEIYGAGGTTTPPTTPAGTYEAESAALSGGAKVNTDHTGYTGTGFVDGYWTQGATTTFTANVSAAGNYDVTLKYANASGSAKTLSVYVNGTKIRQTTLASLANWDTWGTKVETLSLNAGNNTIAYKYEASDSGNVNIDSIAVAPSTSTPVDPEPPITPPTGSNIAIGKAISASSNTQAFVAANANDNDTNTYWEGGAASSTLTLDLGANQNVTSIVLKLNPSSAWSTRTQTIQVLGHNQSTTTFSNLVSSQSYTFNPATGNSVTIPVTATVKRLQLSITANSGSGAGQIAEFQVYGTPAPNPDLTITGMSWTPASPIETDAVTLNATVKNSGNADAPATTVNFYLNNELVGSSPVGALAAGASSTVSLNVGTKTAATYAVSAKVDESNSIIEQNDANNSYTNASSLVVAPVASSDLVGATTWTPSTPVAGNAIGFMVNLKNQGTIASASGAHGITVVVKNAAGAALQSFSGTYSGAIAAGASVNVTLPGTWTAVNGSYTVTTTVAVDANELTNKQGNNVSTSNLVVYAQRGASMPYSRYDTEDATRGGGATLQTAPTFNQAQIASEASGQSYIALPSNGSSAQWTVRQGQGGAGVTMRFTMPDSTDGMGLNGSLDVYVNGVKVKTVSLTSYYSWQYFSGDMPGDAPSAGRPLFRFDEVHWKLDTPLQPGDTIKIQKGNGDSLEYGIDFLEIEPVPTAIAKPANSLSVTEYGAVANDGQDDLAAFKATVTAAVAAGKSVYIPAGTFNLSSMWEIGSANNMINNITITGAGYWHTNIQFTNPNAAGGGISLRISGQLDFSNVYMNSNLRSRYGQNAIYKGFMDNFGTNSKIHDVWVEHFECGMWVGDYAHTPAIYATGLVVENSRIRNNLADGINYSQGTSNSIVRNSSIRNNGDDGLAVWTSNTNGAPAGVNNTFSYNTIENNWRAGGIAFFGGGGHKADHNLIVDTVGGSGIRMNTVFPGYHFQNNTGITFSDNTLINTGTSQDLYNGERGAIDLEASNDAIKNVTFTNIDIINTQRDAIQFGYGGGFENIVFNNININGTGLDGVTTSRFAGPHKGAAIYTYTGNGSATFNNLTTSNVAYPGLNFIQQGFNLVIQ

SEQ ID NO: 15 - DNA, 3786 bp, Paenibacillus curdlanolyticus
atgcgcaacaagtatgtcacatggacgctcgccctgacgatgctattttcgagcttcttccttgcagtaggtcccaacaaggtcgttcacgcagcaggcggaacgaatttagcgctcggcaaaaacgttacggcaagcggccaatcgcaaacgtatagtcccaacaatgtaaaagacagcaatcaatcgacgtactgggaaagcacgaacaatgcattcccgcaatggattcaagtagacttaggcgcaacgacgagcattgaccaaatcgtactgaagctgcccgctggatggggtacgcgtacgcaaacgttagctgttcaaggaagcacggacggttcctcgttcacgaatatcgtgggctccgcaggctatgtatttaatcctgctgttgccaataacgccgttacgattaacttctctgctgcaagcacgcgttatgttcgtctgaacgtaacagcgaacacggcttggccagcagcgcagctgtccgaattcgagatttatggcgctggcggcacgacgacgcctccaacaacgccagcaggcacatatgaagctgaatccgcagcattgtccggcggtgcgaaagtgaacacggatcataccggctacacgggtacgggctttgttgacggctactggacacaaggcgcgacaacgacgttcacggctaacgtgtccgcagctggcaactatgacgttacattgaaatatgccaacgcaagcggcagtgccaagacgctaagcgtttacgtcaacggcacgaagattcgccagacgacgctggcaagcctggcaaactgggacacttggggcacgaaggttgagacgctgagcttgaatgccggcaataatacgattgcatacaagtatgaggctagcgactcgggcaacgtgaatatcgactccattgccgtggcgccatcgacttcgacaccggtagatccagaaccgccgatcacgccgccaacgggcagcaatatcgcaatcggcaaagcgatcagcgcatcttcgaatacgcaagcattcgtagctgccaacgcgaacgataacgatacgaacacgtactgggaaggcggagctgcatcgagcacgctgacgctggatcttggcgcgaaccaaaatgtaacctcgatcgtgctgaagctgaatccttcttcggcatggagcacgcgtacgcaaacgatccaagtgcttggccacaaccaaagcacgacgacgttcagcaatctggtatcttcgcaatcgtatacgttcaatcctgcaacgggcaactccgtgacgattccggttacggcaacagttaagcgcttgcagctgagcattacggcgaactcgggttccggcgctggtcaaattgcggaattccaagtgtatggaacgccggcaccaaacccagacctgacgatcacaggcatgtcctggacgcctgcttcgccaattgaaacggatgcagttacgctgaatgcaacggttaaaaacagcggaaatgcagacgctcctgcaacgacggtaaacttctacctgaacaatgagctcgtaggctcctcgccagttggcgcacttgctgcaggcgcttcctcgacggtttcgctgaatgttggtacgaaaacggctgcaacttatgcagttagcgcgaaagtcgatgagagcaattcgattatcgagcaaaatgatgcgaacaacagttatacgaacgcatcctcgctcgtcgtcgctcctgtcgcaagctctgacttggttggcgcgacgacgtggacgcctagcacgccggttgccggcaatgcaattggcttcatggtaaatcttaaaaaccaaggaacgattgcatctgcaagcggcgcgcatggcattacagttgtcgtgaaaaatgccgcaggcgctgcgctccaatcgttcagcggcacctacagcggagcaatcgcagctggcgcatccgttaacgtaaccctgccaggtacgtggacggctgtgaatggcagctacacggtaacgacaacggttgctgtcgatgctaacgagctgacgaacaaacaagggaacaacgtaagcacttcgaacctcgttgtttatgcacaacgtggcgcaagcatgccttacagccgttatgacacggaagacgctacacgtggcggcggtgcaacgctgcaaaccgcaccaaccttcaaccaagcgcaaatcgcttcggaagcatccggacaaagctatatcgcgctgccttcgaacggctcctccgcacaatggacggtccgtcaaggacaaggcggagctggcgttacgatgcgcttcacgatgccggattcgactgacggtatgggtttgaacggttcgctcgacgtttatgtcaacggcgttaaagtaaaaacggtatcgctcacgtcctactacagctggcagtatttctcgggcgatatgcctggcgatgcgccgtccgctggccgtccgttgttccgctttgacgaagtacactggaagcttgacacgcctcttcaaccaggcgacacgatcaaaatccaaaaaggcaacggagatagcctggaatacggcattgacttcctcgaaatcgagccggttccaacagcaatcgctaaacctgccaactcgctttccgttacggagtatggcgctgtagcaaacgatggccaagacgaccttgccgcattcaaagcaacggttacggctgcagttgctgctggcaaatccgtttacattcctgctggcacgttcaatctgagcagcatgtgggaaatcggatcggctaacaacatgatcaacaacattacgattacaggcgcaggctactggcatacgaacattcaattcacgaatccgaatgcagcaggcggcggcatttcgctccggatttccggacagcttgatttcagcaatgtttacatgaactccaacctgcgttcgcgttatggtcaaaatgcgatttacaaaggcttcatggacaacttcggcacaaactccaaaatccatgacgtatgggttgagcacttcgagtgcggcatgtgggtaggcgattacgcgcatacgccagcgatctatgcaacgggtcttgtcgttgaaaacagccggattcgcaacaaccttgcagacggcatcaactactcgcaaggcacgagcaattcgatcgtacgcaacagcagtatccgcaataacggtgatgacggtctggcggtttggacgagtaacacgaatggcgcgccagcaggcgtgaacaacacgttctcgtacaacacgatcgaaaacaactggcgtgcaggcggtatcgcattcttcggcggcggcggccacaaggctgaccacaacctgatcgttgatacggttggcggctccggcatccggatgaacacggtattcccaggctaccacttccaaaacaacacgggtattacgttctccgacaacacgctgatcaacacaggcacaagccaagatttgtacaacggcgagcgcggtgcgatcgatctcgaagcatcgaacgatgcaatcaagaacgtcacgttcacgaacatcgacatcatcaacacccagcgcgatgcgatacaattcggctacggcggcggattcgagaacatcgtatttaacaacattaacattaacggtacggggcttgacggcgttacaacctcacggtttgctggaccgcataaaggtgctgcaatctacacgtacacgggcaatggctctgcaacgttcaataacctgacgacgagcaacgtggcatatccaggcttgaatttcattcagcaaggctttaatctggtgatccagtag

SEQ ID NO: 16 - protein, 1291 aa, Paenibacillus sp. strain RM1
MRCKFVAWSLVTAMLMASLLTAVGPFGPASAAGGPNLTPGKPITASGQSQTYSPQNVKDGNQNTYWESTNNAFPQWIQVDLGASTGIDQIVLKLPASWEARTQTLAVQGSLNGSTFTDIVGSANYVFSPSVGNNTVTINFTATSTRYVRLYVTANTGWPAAQLSEFEIYGSGDQTPAPDTYQAESAALSGGAKVNTDHAGYIGTGFVDGYWTQGATTTFSVNAPTAGNYDVTLRYGNATGSNKTVSLYVNGAKIRQTTLPSLPNWDSWSSKTETLNLNAGSNTIAYKYDPGDSGNVNLDQITVEASTSTPTPTPSPTPTPTPTPTPTPTPTPTPTPTPTPTPTPTPTPTPTPPPGGNIAIGKSISASSHTQTYVAENANDNDVNTYWEGGGNPSTLTLDLGANYNITSIVLKLNPSSIWAARTQTIQVLGHDQNTTTFSNLVSAKSYSFDPASGNTVTIPVTATVKRLQLNITSNSGAPAGQVAEFQVFGTPAPNPDLTITGMSWSPSSPVETDAITLNATVKNNGNASAAATTVNFYLNNELAGSAPVAALAAGASATVPLNVGAKTAATYAVGAKVDESNAVIELNESNNSYTNPASLVVAPVSSSDLVGTVSWTPSTPIANNAVSFNVNLKNQGTIASAGGSHGVTVVLKNASGSTVQTFSGSYTGSLAPGASVNITLPGWLTAAAGSYTVTATVAADANELPIKQANNANTASLTVYSARGASMPYSRYDTEDATLGGGATLKSAPTFDQALTASEATGQLYAALPSNGSYLQWTVRQGQGGAGVTMRFTMPDSADGMGLNGSLDVYVNGTKVKTVSLTSYYSWQYFSGDMPGDAPSAGRPLFRFDEVHWKLDTPLKPGDTIRIQKNNGDSLEYGVDFIEIEPVPAAISRPANSVSVTDYGAVPNDGQDDLTAFKAAVNAAVASDKILYIPEGTFHLGNMWEIGSVSNMIDHITITGAGIWYTNIQFTNANPASGGISLRITGKLDFSNVYLNSNLRSRYGQNAVYKGFMDNFGTNSVIRDWIVEHFECGFWVGDYGHTPAIRASGLLIENSRIRNNLADGVNFAQGTSNSTVRNSSLRNNGDDALAVWTSNTNGAPEGVNNTFSYNTIENNWRAGGIAFFGGSGHKADHNYIVDCVGGSGIRMNTVFPGYHFQNNTGIVFSDTTIVNCGTSKDLYNGERGAIDLEASNDAIRNVTFTNIDIINSQRDAIQFGYGGGFTNIVFNNININGTGLDGVTTSRFSGPHLGAAIFTYTGNGSATFNNLRTSNIAYPNLYYIQSGFNLIINN

SEQ ID NO: 17 - DNA, 5090 bp, Paenibacillus sp. strain RM1
cccgggtaccagacctatcgggaaaaacgcgagcggcccttcgcgccttatgcgctacggacggtgctggcgggcggtttgtttttcatcatcattcccctgatgatctacacggcatcgtatatcccgtttttgctcgtgccgggtcccggacacgggttgaaagacgtcgtctccgcccagaagttcatgttcaattatcatagccggcttaacgccacccacccattctcgtcgctgtggtgggagtggcctctcatccgcaagccgatctggtattacggagccgcggaattggcgccgggaaaaatggcgagcatcgtgggcatgggcaatccggcggtgtggtggacgggaacgattgcggtaatcgcggcccttcgctcggcctggaagaagcgggaccggagcatgaccgtcgtcttcgttggaatcgcctcgtcttatcttccgtgggttttcgtatccagactcacctttatttatcactttttcgcttgcgttccgtttctcgttctttgcatcgtttattggattcgaaaaatggaatagcgtaagccgggatatcggattgcgacgctcctttacgcaggcgcggttctggtgctgttcattttgttttacccgattttgtcggggaccgaaatagacgtttcttacgcggaccgcgttctgaagtggttcggcgggtggatttttcacgggtaagcgagcgttggaagcaaggaagggaaggaagacgagcgtctccttcccgaaatccatccaatatcttgaaattgcatacatttttcgtaagattgcttcttatctgtctccctcccctgttcttataatgggggtatcccaacgaaaggagggtttgtaagcgctgtcagcstgtttgccgaaagttctcgcatttgctgacctacactttgaggaggaggaatttaatgcgctgcaaatttgtcgcatggtcgcttgttacagccatgctgatggccagtttgctgacggctgtaggaccgttcggccccgcttccgccgcgggaggaccgaatctgacgccgggcaaacccattacggcgagcggccaatcccaaacctacagccctcagaacgtaaaagacggcaatcaaaatacgtattgggaaagcacgaacaacgcgttcccgcaatggattcaagtggatttgggcgcaagcacgggcatcgaccaaattgtgctgaagctgcccgcaagctgggaagcgcgcacgcaaacgctggccgttcaaggcagcttgaacggttcgacgttcacggacattgtcggctccgccaattatgtattcagtccgtctgtcgggaacaacacggttacgatcaactttaccgcgaccagcacgcgctacgtgcgcttgtatgtaacggccaacacgggctggccggcggcgcagctgtccgaattcgaaatttacggctccggcgaccagacgccggcgcctgatacgtatcaagccgaatccgcggctctgtccggcggcgcgaaagtcaacacggaccatgccggatatatcggcacgggctttgttgacggttactggacgcaaggcgcgacgacgaccttttcggtcaacgcgccgacggcgggcaactacgatgtaacgctgaggtacggcaacgcaaccggcagcaacaaaacggtaagcctctacgtcaatggagcgaagattcgccagaccacgctgcccagcctgcctaactgggattcatggagcagcaagacggagacgcttaacctgaatgcaggcagcaacaccattgcgtacaaatacgacccgggcgattccggcaacgtcaatcttgaccaaatcacggtcgaagcgtcgacttcaacgcctactcctactccatcccctactcctacacctacgccaacgccgacgcctacgcctacgcctacacccacacctactccgaccccgacgcctacgcctacacctacacctacacctacgccgacgcctcctccgggcggcaacatcgccatcggcaaatcgatttccgcatcctcccacacgcagacgtacgttgcggagaacgcgaacgataacgatgtcaacacgtactgggaaggcggcggcaatccgagcacgctgacgctcgatctcggagcgaactacaatattacgtccatcgtgctgaagctgaacccgtcctcgatatgggctgcgcgtacgcaaacgattcaagtgctcggacacgatcagaacacgacgaccttcagcaatctggtctcggcgaaatcgtactcgttcgatccggcctccggcaatactgtgaccattccggttacggcgacggtgaaacgtttgcagttgaacattacgtcgaactccggcgccccggccggacaagtcgccgagttccaggtgttcggcacgcctgcgccgaatccggacctgacgattaccggcatgtcctggtcgccttcttctccggttgagaccgacgccattacgctaaacgcaacggtgaagaacaacgggaatgccagcgccgcggcgaccaccgtcaatttctacctgaacaacgagctggcgggttccgcgccggtagccgcgctggcggcaggcgcttcggcaacggtgccgctgaatgtcggcgcgaaaaccgccgcgacatacgcggtcggcgccaaagtagacgagagcaacgcggtcatcgagctgaacgagtcgaacaacagctacacgaatccggcttcactcgttgtggcccccgtttccagctcggatctggtgggcacggtttcgtggacgccgagcactccgattgccaacaatgccgtttcttttaacgtaaatcttaaaaatcaaggaacgattgcttccgccggcgggtctcacggcgtgacggtcgtgcttaaaaatgcttccggttcgaccgttcaaacgttcagcggttcctataccggcagcctggctccgggagcgtccgtcaacatcacccttccggggacctggacggcggcagccggcagctacacggtaacggccaccgttgcggcagacgccaacgaacttccgatcaagcaagccaacaacgcgaacaccgcaagcctgaccgtatattccgcccgcggcgcgagcatgccgtacagccggtatgacaccgaggacgccaccctcggcggcggcgccacgctgaagtccgcgccgacattcgatcaggcgcttacggcatcggaagccaccggccaactctatgcggcgctgccctcgaacggctcctatcttcaatggaccgtcagacagggtcagggcggcgcaggcgtgacgatgagatttacgatgcccgactcggcggacggcatgggattaaacggttcgctagacgtttacgtcaacggcaccaaagtcaaaaccgtatcgctgacctcctactacagctggcagtatttctcgggcgatatgcccggagacgctcccagcgcgggccgtccgctcttccgctttgacgaagtgcactggaagctggatactccgctcaaacccggagacacgattcgcatccagaagaacaacggcgacagcctggaatacggtgtcgactttattgaaatcgaaccggttccggctgcgatctcccgtccggccaactcggtttccgtaacggattacggcgctgtgccgaacgacggacaggacgatctcaccgcctttaaagccgccgtaaacgcggcggtcgcatccgacaagatcttgtacattccggaaggaacgttccacctcggcaacatgtgggagatcggttccgtcagcaacatgatcgatcacattacgattacgggagccggtatctggtatacgaacatccagtttaccaacgccaatccggcgtccggcggcatctcgctccggattacgggcaagcttgatttcagcaacgtgtacctcaactccaatttgcggtcgcggtatggtcaaaatgcggtttacaaaggctttatggacaacttcgggaccaattccgtcatccgcgacgtctgggtcgagcacttcgaatgcggcttctgggtcggggactacgggcatacgccggcgatccgcgcgagcgggctgctgattgaaaacagccgaatccgcaacaacctggccgatggcgtcaacttcgcccaagggaccagcaattcgaccgtacgcaacagcagcctgcgcaacaacggcgacgacgcccttgccgtatggacgagtaatacgaacggcgcgcccgaaggcgtaaacaataccttctcgtacaacaccatcgaaaacaactggcgcgcgggaggcatcgccttcttcggaggaagcggacacaaggccgaccacaactacatcgtcgactgcgtcggcggttccggcatccggatgaacaccgtgttccccggataccacttccagaacaataccggcattgtgttctcggacacgaccatcgtcaactgcggcacgagcaaagacctatacaacggcgaacgcggcgccatcgatctggaagcttcgaacgacgccatccggaacgtgacgtttaccaacatcgatattatcaactctcagcgcgatgcgatccagttcggttacggcggcggcttcaccaacatcgtgttcaacaacatcaacattaacggaaccggtcttgacggcgtaaccacctcgcggttctcgggaccgcatctgggcgcggcgatcttcacctataccggcaacggctccgccacgttcaacaatctgaggaccagcaatatcgcttaccccaatctgtattacatccagagcgggttcaatctgatcatcaataattagatatctgggcccgtctgcgggggaggaactcttcggagctcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaactgtcgtgccagctgcattaatgaatcggccaacgcgcggggagaggcsgtttkcgtattgggcgccctt

SEQ ID NO: 18 - protein, 635 aa, Trichoderma harzianum
MLGVFRRLRLGALAAAALSSLGSAAPANVAIRSLEERASSADRLVFCHFMIGIVGDRGSSADYDDDMQRAKAAGIDAFALNIGVDGYTDQQLGYAYDSADRNGMKVFISFDFNWWSPGNAVGVGQKIAQYANRPAQLYVDNRPFASSFAGDGLDVNALRSAAGSNVYFVPNFHPGQSSPSNIDGALNWMAWDNDGNNKAPKPGQTVTVADGDNAYKNWLGGKPYLAPVSTWVFNHFGPEVSYSKNWVFPSGPLIYNRWQQVLQQGFPRVEIVTWNDYGESHYVGPLKSKQFHDGNSKWVNDMPHDGFLDLSKPFIAAYKNRDTDISKYVQNEQLVYWYRRNLKALDCDATDTTSNRPANNGSGNYFEGRPDGWQTMDDTVYVAALLKTAGSVTVTSGGTTQTFQANAGANLFQIPASIGQQKFALTRNGQTVFSGTSLMDITNVCSCGIYNFNPYVGTIPAGFDDPLQADGLFSLTIGLHVTTCQAKPSLGTNPPVTSGPVSSLPASSTTRASSPPPVSSTRVSSPPVSSPPVSRTSSAPPPPGNSTPPSGQVCVAGTVADGESGNYIGLCQFSCNYGYCPPGPCKCTAFGAPISPPASNGRNGCPLPGEGDGYLGLCSFSCNHNYCPPTACQYC

SEQ ID NO: 19 - DNA, 2041 bp, Trichoderma harzianum
atgttgggcgttttccgccgcctcaggctcggcgcccttgccgccgcagctctgtcttctctcggcagtgccgctcccgccaatgttgctattcggtctctcgaggaacgtgcttcttctgctgaccgtctcgtattctgtcatttcatgattgggatcgtgggtgaccgtggcagctcggcagattatgatgacgatatgcaacgtgccaaagccgctggcattgacgccttcgccctgaacatcggcgttgacggctataccgaccagcagctcggctatgcctatgactctgccgatcgtaatggcatgaaagtcttcatttcatttgatttcaactggtggagccccggcaatgcagttggtgttggccagaagattgcgcagtatgccaaccgccctgcccagctgtatgtcgacaaccggccattcgcctcttccttcgccggtgacggtctggatgtaaatgcgttgcgctctgctgcaggctccaacgtttactttgtgcccaacttccaccctggtcaatcttccccctccaacattgatggcgcccttaactggatggcctgggataatgatggaaacaacaaggcacccaagccgggccagactgtcacagtggcagacggtgacaacgcttataagaattggttgggtggcaagccttacctggcgcctgtctcaacttgggttttcaaccatttcgggcccgaagtttcatattccaagaactgggttttcccaagtgggcctctgatctataaccggtggcaacaagtcttgcagcaagggttcccaagggttgagatcgttacctggaatgactacggggaatctcactacgtcggtcccctgaagtctaagcaatttcatgatgggaactccaaatgggtcaatgatatgccccacgatggattcctggatctttcgaagccgttcatagccgcatataaaaacagggataccgacatctccaagtatgttcaaaatgagcagcttgtttactggtaccgccgcaacttaaaggcactggactgtgacgccaccgacacaacctctaaccgcccggctaacaatggaagcggcaattactttgagggacgccccgatggttggcaaactatggatgatacggtttacgtggcggcacttctcaagactgccggtagcgtcacggtcacgtctggtggcaccactcaaacgttccaggccaacgccggagccaatctcttccaaatcccggccagcatcggccagcaaaagtttgctctgactcgtaacggtcagaccgtctttagcggaacctcattgatggatatcaccaacgtttgctcttgcggtatctacaacttcaacccatatgttggcaccattcctgccggctttgacgaccctcttcaggctgacggtcttttctctttgaccatcggattgcacgtcacaacttgtcaggccaagccatctcttggaactaaccctcctgtcacttccggccctgtgtcctcgcttccagcttcctccaccacccgcgcatcctcgccgcctcctgtttcttcaactcgtgtctcttctccccctgtctcttcccctccagtttctcgcacctcttctgcccctccccctccgggcaacagcacgccgccatcgggtcaggtttgcgttgccggcaccgttgccgacggcgagtctggcaactacatcggcctgtgccaattcagctgcaactacggttactgcccaccaggaccgtgtaagtgcaccgcctttggtgctcccatctcgccaccggcatccaacggccgcaacggctgccctctgccgggagaaggcgatggttatctgggcctgtgcagtttcagttgtaaccataattactgcccgccaacggcatgtcaatactgctaggagggatcaatctcagtatgagtatatggaggctgctgaaggaccaattagctgttcttatcggcagacgaaacccatagagtaagaagttaaataaaatgcaattaatgtgttttcaaaaaaaaaaaaaaaa

SEQ ID NO: 20 - protein, 635 aa, Trichoderma harzianum; identical to SEQ ID NO: 18

SEQ ID NO: 21 - DNA, 2041 bp, Trichoderma harzianum; identical to SEQ ID NO: 19

SEQ ID NO: 22 - protein, 608 aa, Penicillium minioluteum
MATMLKLLALTLAISESAIGAVMHPPGNSHPGTHMGTTNNTHCGADFCTWWHDSGEINTQTPVQPGNVRQSHKYSVQVSLAGTNNFHDSFVYESIPRNGNGRIYAPTDPPNSNTLDSSVDDGISIEPSIGLNMAWSQFEYSHDVDVKILATDGSSLGSPSDVVIRPVSISYAISQSDDGGIVIRVPADANGRKFSVEFKTDLYTFLSDGNEYVTSGGSVVGVEPTNALVIFASPFLPSGMIPHMTPDNTQTMTPGPINNGDWGAKSILYFPPGVYWMNQDQSGNSGKLGSNHIRLNSNTYWVYLAPGAYVKGAIEYFTKQNFYATGHGILSGENYVYQANAGDNYIAVKSDSTSLRMWWHNNLGGGQTWYCVGPTINAPPFNTMDFNGNSGISSQISDYKQVGAFFFQTDGPEIYPNSVVHDVFWHVNDDAIKIYYSGASVSRATIWKCHNDPIIQMGWTSRDISGVTIDTLNVIHTRYIKSETVVPSAIIGASPFYASGMSPDSRKSISMTVSNVVCEGLCPSLFRITPLQNYKNFVVKNVAFPDGLQTNSIGTGESIIPAASGLTMGLNISNWTVGGQKVTMENFQANSLGQFNIDGSYWGEWQIS

SEQ ID NO: 23 - DNA, 3629 bp, Penicillium minioluteum
ggcatagtaatcccgacagccgagtatgatggagcttcttcggataatgatagcgccaccagaccttgcttgagctggagagctaaaacattaaacgccacacgaccaacactctcattagttgcgatagatgatgctcggagctgttgaaactcagaaattccttctatgcggggtctccaagatcgatcctgggggatgtgaatactacggtggacctaattgacgccttgacaggtgatgttaagcgaaccaaggaagaataatctggggctagatgaagatgttgagctgtaaggtacggtacgttcctattggctttatcggagcttctccgggttactcagtctttccgggagcatgatcatttttgtattgtccaatagtaagcagaaactgagagccaccacaaactcaaaacctcggtagcgaagtttcccggaaccagtcaggattctcagaaactgtgctcgtgttgcggggaatccgcattctacgtcgtctggagcaaggaaatgttcgtgctggattgaggaggataggtaggttggagaatctcttcagctaaccaatctataagcatgctccggtaacctttagagtttcacattcaacgtaatttccaagatagccagagcgtccttgaattactatgtagaaatcctaaaatttcccctgtaaaatgcaagtcaacgagatgcgtgccctcaatgtctctcggcgctaccccggaaatgatgcataaggccaagaatgtcacccggtaactttttcttcagaatatcctaagatttccatcaaacacagtcgaataggtcaatgctcgcgagagactttctgccttcactctacgtcctactcatagaagttcaacggctcaattccggggtaatctagagtttggacctcaagggagatgttgcaacaaattgtactagaacgatgcgcttgctttccaatacagtagttgacttcatatagcttccaacaaaagggatggggatgaaggctctatagcgagaagtctataagaaagtgtcctcatacctgtatctctcagtcgttcgagaacaatcccggaaactatcttatcttgcgagaaagaagacaatatctcaaacttatggccacaatgctaaagctacttgcgttgacccttgcaattagcgagtccgccattggagcagtcatgcacccacctggcaattctcatcccggtacccatatgggcactacgaataatacccattgcggcgccgatttctgtacctggtggcatgattcaggggagatcaatacgcagacacctgtccaaccagggaacgtgcgccaatctcacaagtattccgtgcaagtgagcctagctggtacaaacaattttcatgactcctttgtatatgaatcgatcccccggaacggaaatggtcgcatctatgctcccaccgatccacccaacagcaacacactagattcaagtgtggatgatggaatctcgattgagcctagtatcggccttaatatggcatggtcccaattcgagtacagccacgatgtagatgtaaagatcctggccactgatggctcatcgttgggctcgccaagtgatgttgttattcgccccgtctcaatctcctatgcgatttctcagtctgacgatggtgggattgtcatccgggtcccagccgatgcgaacggccgcaaattttcagttgagttcaaaactgacctgtacacattcctctctgatggcaacgagtacgtcacatcgggaggcagcgtcgtcggcgttgagcctaccaacgcacttgtgatcttcgcaagtccgtttcttccttctggcatgattcctcatatgacacccgacaacacgcagaccatgacgccaggtcctatcaataacggcgactggggcgccaagtcaattctttacttcccaccaggtgtatactggatgaaccaagatcaatcgggcaactcggggaagttaggatctaatcatatacgtctaaactcgaacacttactgggtctaccttgcccccggtgcgtacgtgaagggtgctatagagtattttaccaagcagaacttctatgcaactggtcatggtatcctatcgggtgaaaactatgtttaccaagccaatgccggcgacaactacattgcagtcaagagcgattcaaccagcctccggatgtggtggcacaataaccttgggggtggtcaaacatggtactgcgttggcccgacgatcaatgcgccaccattcaatactatggatttcaatggaaattctggcatctcaagtcaaattagcgactataagcaggtgggagccttcttcttccagacggatggaccagaaatatatcccaatagtgtcgtgcacgacgtcttctggcacgtcaatgatgatgcaatcaaaatctactattcgggagcatctgtatcgcgggcaacgatctggaaatgtcacaatgacccaatcatccagatgggatggacgtctcgggatatcagtggagtgacaatcgacacattaaatgttattcacacccgctacatcaaatcggagacggtggtgccttcggctatcattggggcctctccattctatgcaagtgggatgagtcctgattcaagaaagtccatatccatgacggtttcaaacgttgtttgcgagggtctttgcccgtccctattccgcatcacaccccttcagaactacaaaaattttgttgtcaaaaatgtggctttcccagacgggctacagacgaatagtattggcacaggagaaagcattattccagccgcatctggtctaacgatgggactgaatatctccaactggactgttggtggacaaaaagtgactatggagaactttcaagccaatagcctggggcagttcaatattgacggcagctattggggggagtggcagattagctgaattccagctctcggagcgcgtgagtgcttctacccgctcctttacccttgtcgagagataaaggcataagttagctcatgtgaaggcgatttcagttcattctctctttttggagcttatttcctgttcgaccaattgtgacaccaacttgcctttcaaaagacgtggacgatatgtgtacggtaatcagtcaaatgaacgtcaacattcatttaataaggacatttccaggtttccttactctgtcgattatgcctaactcgggttgatgtcttgtcaggatggaaaatctcgttgtgtacttccagtgaaatgggcagggctaagccctaaaccctaacgcatacaatttgtaggcacctacccatgtaagttcacacccagtcgacttataagtctagatatttatgctatgcaggctctggaatgatttacattccatgctatacatagttatttgcaagaatttgcagacgagataaaaatcaatggacgaataatcacgcattactccacaggctcatgccacggagcaagggttcccccgaatctaggccagaccgggatgatattcaaccgattctttttgcagtaactatctccgtacgagctgcacgagctaaacggattatataaaggtgctaactgagcattggatccgtcagttatatgaaatgca

SEQ ID NO: 24 - protein, 608 aa, Penicillium aculeatum
MATMLKLLTLALAISESAIGAVLHPPGSSHPSTRTDTTNNTHCGADFCTWWHDSGEINTQTPVQPGNVRQSHKYSVQVSLAGANNFQDSFVYESIPRNGNGRIYAPTDPPNSNTLDSSVDDGISIEHSIGLNMAWSQFEYSQDVDIKILAADGSSLGSPSDVVIRPVSISYAISQSDDGGIVIRVPADANGRKFSVEFKNDPYTFLSDGNEYVTSGGSVVGVEPTNALVIFASPFLPSGMIPHMTPDNTQTMTPGPINNGDWGSKSILYFPPGVYWMNQDQSGNSGKLGSNHIRLNSNTYWVYFAPGAYVKGAIEYFTKQNFYATGHGVLSGENYVYQANAGENYVAVKSDSTSLRMWWHNNLGGGQTWYCVGPTINAPPFNTMDFNGNSGISSQISDYKQVGAFFFQTDGPEIYPNSVVHDVFWHVNDDAIKIYYSGASVSRATIWKCHNDPIIQMGWTSRDISGVTIDTLNVIHTRYIKSETVVPSAIIGASPFYASGMSPDSSKSISMTVSNVVCEGLCPSLFRITPLQNYKNFVVKNVAFPDGLQTNSIGTGESIIPAASGLTMGLDISNWSVGGQKVTMQNFQANSLGQFDIDGSYWGEWQIN

SEQ ID NO: 25 - DNA, 1888 bp, Penicillium aculeatum
atggccacaatgctaaagctacttacgttggcccttgcaattagcgagtctgccattggagcagtcctgcacccacctggcagttctcatcccagtacccgtacggacactacgaataatacccattgcggtgccgacttctgtacctggtggcatgattcaggcgagatcaacacacagacacctgtccaaccggggaacgtgcgccaatctcacaagtattccgtacaagtgagcctagctggtgcgaacaactttcaggactcctttgtatatgaatcgatccctcggaacggaaatggtcgcatctatgctcccaccgatccacccaacagcaacacactagattcaagtgttgatgatggaatctcgattgaacatagtattggcctcaatatggcatggtcccaattcgagtacagccaggatgtcgatataaagatcctggccgctgatggctcatcgttgggctcaccaagtgatgttgttattcgccccgtctcaatctcctatgcaatttctcaatccgacgatggcggaattgtcattcgggtcccagccgatgcgaacggccgcaaattttcagtcgagttcaaaaatgacccgtacacgttcctctctgacggcaacgagtacgtcacatcgggaggcagcgttgtcggcgttgagcctaccaacgcacttgtgatcttcgcaagcccgtttcttccgtcaggcatgattcctcatatgacacccgacaacacgcagaccatgacaccaggacctatcaataacggcgactggggctccaagtcaattctttatttcccaccgggcgtatactggatgaaccaagatcaatcaggcaactcggggaaattaggatctaatcatatacgcctgaactcgaacacctactgggtctactttgccccaggtgcgtacgtgaagggtgctatagagtatttcaccaagcagaacttctatgcaactggtcatggtgtcctatcgggtgaaaactatgtttaccaagccaatgctggcgaaaactacgttgcggtcaagagcgattcgactagcctccggatgtggtggcacaataacctgggaggtggacaaacatggtactgcgttgggcctacgatcaatgcgccgccatttaacacaatggatttcaatggaaattccggtatctcaagtcaaattagcgactataagcaggtgggagctttcttctttcagacggatggaccagaaatttatcccaatagtgtcgtgcacgacgtcttctggcatgtcaatgatgatgcaatcaaaatctactattccggagcatctgtctcgcgggcaacgatctggaaatgtcacaacgatccaatcatccagatgggatggacgtctcgggatatcagtggagtgacaatcgacacattgaatgtcatccacacccgctacatcaagtcggagacggtggtgccttcggctatcattggggcttctccattctatgcaagtgggatgagtcctgattcaagcaagtctatatccatgacggtttcaaacgttgtctgcgagggactttgcccgtctctgttccgaatcacacctttacagaactacaagaattttgttgtcaaaaatgtggctttcccagatgggctacagacgaatagtattggcacgggagaaagcattattccagccgcatctggtctaacgatgggactggatatctccaactggtctgttggtggtcagaaggtgactatgcagaactttcaagccaatagtctggggcaattcgacattgacggcagctattggggggagtggcagattaactagctgaataatattgcagctttcagggcgcatgagtgcttgtacccgctcctttacccttgtc

SEQ ID NO: 26 - protein, 615 aa, Penicillium funiculosum
MATMLKLLALTLAISESAIGAVMHPPGVSHPGTHTGTTNNTHCGADFCTWWHDSGEINTQTPVQPGNVRQSHKYSVQVSLAGTNNFHDSFVYESIPRNGNGRIYAPTDPSNSNTLDSSVDDGISIEPSIGLNMAWSQFEYSQDVDIKILATDGSSLGSPSDVVIRPVSISYAISQSNDGGIVIRVPADANGRKFSVEFKNDLYTFLSDGNEYVTSGGSVVGVEPTNALVIFASPFLPSGMIPHMKPHNTQTMTPGPINNGDWGAKSILYFPPGVYWMNQDQSGNSGKLGSNHIRLNSNTYWVYLAPGAYVKGAIEYFTKQNFYATGHGVLSGENYVYQANAGDNYVAVKSDSTSLRMWWHNNLGGGQTWYCVGPTINAPPFNTMDFNGNSGISQISDYKQVGAFFFQTDGPEIYPNSVVHDVFWHVNDDAIKIYYSGASVSRATIWKCHNDPIIQMGWTSRDISGVTIDTLNVIHTRYIKSETVVPSAIIGASPFYASGMSPDSSKSISMTVSNVVCEGLCPSLFRITPLQNYKNFVVKNVAFPDGLQTNSIGTGESIIPAASGLTMGLNISSWTVGGQKVTMENFQANSLGQFNIDGSYWGEWQISRISSSQSA

SEQ ID NO: 27 - DNA, 2225 bp, Penicillium funiculosum
atggccacaatgctaaagctacttgcgttgacccttgcaattagcgagtccgccattggagcagtcatgcacccacctggcgtttctcatcccggtacccatacgggcactacgaataatacccattgcggcgccgacttctgtacctggtggcatgattcaggggagatcaacacgcagacacctgtccaaccagggaacgtgcgccaatctcacaagtattccgtgcaagtgagtctagctggtacaaacaactttcatgactcctttgtatatgaatcgatcccccggaacggaaatggtcgcatctatgctcccaccgatccatccaacagcaacacattagattcaagcgtggatgatggaatctcgattgagcctagtatcggcctcaatatggcatggtcccaattcgagtacagccaggatgtcgatataaagatcctggcaactgatggctcatcgttgggctcaccaagtgatgttgttattcgccccgtctcaatctcctatgcgatttctcagtccaacgatggcgggattgtcatccgggtcccagccgatgcgaacggccgcaaattttcagtcgaattcaaaaatgacctgtacactttcctctctgatggcaacgagtacgtcacatcgggaggtagcgtcgtcggcgttgagcctaccaacgcacttgtgatcttcgcaagtccgtttcttccttctggcatgattcctcatatgaaaccccacaacacgcagaccatgacgccaggtcctatcaataacggcgactggggcgccaagtcaattctttacttcccaccaggtgtatactggatgaaccaagatcaatcgggcaactcgggtaaattaggatctaatcatatacgtctaaactcgaacacttactgggtctaccttgcccccggtgcgtacgtgaagggtgctatagagtatttcaccaagcaaaacttctatgcaactggtcatggtgtcctatcaggtgaaaactatgtttaccaagccaatgctggcgacaactatgttgcagtcaagagcgattcgaccagcctccggatgtggtggcacaataaccttgggggtggtcaaacatggtactgcgttggcccgacgatcaatgcgccaccattcaacactatggatttcaatggaaattctggcatctcaagtcaaattagcgactataagcaggtgggagccttcttcttccagacggatggaccagaaatctatcccaatagtgtcgtgcacgacgtcttctggcacgtcaatgatgatgcaatcaaaatctactattcgggagcatctgtatcgcgggcaacgatctggaaatgtcacaatgacccaatcatccagatgggatggacatctcgggatatcagtggagtgacaatcgacacattaaatgttattcacacccgctacatcaaatcggagacggtggtgccttcggctatcattggggcctctccattctatgcaagtgggatgagtcccgattcaagcaagtccatatccatgacggtttcaaacgttgtttgcgagggtctttgcccgtccctgttccgcatcacacccctacagaactacaaaaattttgttgtcaaaaatgtggctttcccagatgggctacagacaaatagtattggcacaggagaaagcattattccagccgcatctggtctaacgatgggactaaatatctccagctggactgttggtggacaaaaagtgacaatggagaactttcaagccaatagcctggggcagttcaatattgacggcagctattggggggagtggcagattagtcgaatttccagctctcagagcgcgtgagtgcttctacccgctcctttacccttgtcgaaggatcaaggcataagttagctcatgtgaaggcgatttcagttcattctctcttttttggagctcatttccttttcgaccaattgtgacaccaaattgccatgtgtactgtaattggtcaaatgaacgttaaccttcgatttaatatggacatttccaggtttccttactctgtcgattatgcctaactcgggttgatgtcttgtcaggatgaaaatctcgttgtcatgtacttcgagtgaaatgggcagggctaacccctaagccctaacgcccaatcgacttataagtctagatgtttatgctatgcaggctctggaatgatttacattccatgctataca

SEQ ID NO: 28 - protein, 21 aa, Artificial Sequence
  CSP
SGSLSTFFRLFNRSFTQALGK

SEQ ID NO: 29 - DNA, 31 bp, Artificial Sequence
  PCR primer 16S-F
cagcagccgcggtaatacagaggatgcaagc

SEQ ID NO: 30 - DNA, 31 bp, Artificial Sequence
  PCR primer aadA-R
ccgcgttgtttcatcaagccttacggtcacc

SEQ ID NO: 31 - DNA, 31 bp, Artificial Sequence
  PCR primer atpB-R
gaattaaccgatcgacgtgctagcggacatt

SEQ ID NO: 32 - DNA, 28 bp, Artificial Sequence
  PCR primer UTR-F
aggagcaataacgccctcttgataaaac

SEQ ID NO: 33 - DNA, 28 bp, Artificial Sequence
  PCR primer 23S-R
tgcacccctacctcctttatcactgagc

SEQ ID NO: 34 - DNA, 2553 bp, Streptococcus mutans
atggaacagtcaaataggcaaacggctgaaccagctattaggtcaaatgaaacggtggattcggccattaactcttttcaagagacagaccttaaggtgcaagagaaggaggatgctgcggctgcagtacagacagaaccggcgtcaatagattctaatgaacagggacaatcggtctctgcaaatactaacacacaatctcaagcgaagaaactttctaacaattcccatcaggagccaatgcaaatggcatctgccgccaataaagaaagggttgtgctagaaactgcacagaatcaaaagaatggcaacatgataaatctgacaacagataaagcagtctaccaggcgggagaggctgttcatttgaaccttactttaaacaatacaacatctttagcccaaaatattacagctactgctgaggtttattcccttgaaaataaattaaagacacttcagtatacgaagtatcttctgcctaatgaaagttatacaactcaaaaaggtgaattcgttattcctgcaaactccttagctaataatcgcggttatcttttgaaggttaacatatcagatagccaaaataatattttagagcagggcaatcgggctattgcggttgaggatgactggcgtacctttccgcgttatgctgctattggaggatctcaaaaagacaataacagtgtcttgactaagaacttaccagattattatcgcgaattagagcagatgaaaaatatgaacattaattcctatttcttctatgatgtttataagtctgctacaaatcctttccctaatgttcctaagtttgatcagtcttggaattggtggagccattcgcaggttgaaacagatgctgttaaagccttggtcaatcgtgtccatcaaactggcgctgttgccatgctctataatatgattttagcacagaatgctaatgaaacggctgttttaccagatactgagtacatctataattatgagactggtggttatggtcaaaatggtcaggtcatgacttactctattgatgataagccgctgcaatattattacaatcctttgagtaaaagttggcaaaattatatttctaatgcaatggctcaagctatgaaaaatggcggttttgatggctggcagggagatacaattggagataatcgtgttctttcccataaccaaaaggacagtcgagatattgctcattcctttatgttatctgatgtctatgctgaatttctcaataaaatgaaggaaaaactgcctcagtattatttaacactcaatgatgttaatggtgaaaatatcagcaaactcgccaacagcaaacaagatgtgatttacaatgaattatggccttttggaacttcagctttggggaaccgtccccaagaaagttatggtgacttgaaagctcgtgttgatcaagttcgccaagcgacagggaaatctttgattgtcggagcttatatggaagagcctaaatttgatgataataggattcctctcaatggtgcagcgcgtgacgttttagcttcagcaacttaccaaacagatgcggttctgctgacaactgcggccattgcggcagcaggaggatatcacatgtctctggctgctctggctaatcctaatgatgggggtggtgtcggtgtcttagaaacagcttattatccaacacaaagcctcaaggtttcgaaagagctcaatcgtaaaaactatcattaccaacaatttattacggcttatgaaaatcttttgcgtgataaagttgaaaatgattctgctgaacctcagactttcactgctaacggtcggcagctatcgcaagatgctttggggatcaatggcgatcaggtttggacttatgccaaaaagggaaacgatttcagaacgattcaattgctcaaccttatgggaattacatccgactggaaaaatgaagatggttatgaaaataataaaacacctgatgagcaaaccaatttattggttacttatcctttgactggtgtgtctatggcagaggctgatcgaatagctaaacaagtctatctgacgtcaccagatgattggctgcaatctagtatgatttctctagcgactcaggtaaaaacgaatgagaatggcgatcctgttctttatattcaagtgccaagactgacgctttgggatatgatttatattaatgaaaccattaaaccagaaacgcctaaagttccagaacagccccaacatcctgctaggacacttgaaccagcaattccgcaaactccagaagcagtcaaccctctcccagtagctaataagcaggcagtagatgaaaataaaaatgagattgtttcagccttaaccggtgaagaaaatgacttgcagttgccaactctttccaaacaatcattgccaatctcccaagcagagttaccgcaaacaggagataacaatgaaacgcgctccaatctcctcaaagtgataggtgctggtgcgcttctaatcggcgctgcaggattattaagcttgataaagggtagaaaaaatgattga

SEQ ID NO: 35 - protein, 850 aa, Streptococcus mutans
MEQSNRQTAEPAIRSNETVDSAINSFQETDLKVQEKEDAAAAVQTEPASIDSNEQGQSVSANTNTQSQAKKLSNNSHQEPMQMASAANKERVVLETAQNQKNGNMINLTTDKAVYQAGEAVHLNLTLNNTTSLAQNITATAEVYSLENKLKTLQYTKYLLPNESYTTQKGEFVIPANSLANNRGYLLKVNISDSQNNILEQGNRAIAVEDDWRTFPRYAAIGGSQKDNNSVLTKNLPDYYRELEQMKNMNINSYFFYDVYKSATNPFPNVPKFDQSWNWWSHSQVETDAVKALVNRVHQTGAVAMLYNMILAQNANETAVLPDTEYIYNYETGGYGQNGQVMTYSIDDKPLQYYYNPLSKSWQNYISNAMAQAMKNGGFDGWQGDTIGDNRVLSHNQKDSRDIAHSFMLSDVYAEFLNKMKEKLPQYYLTLNDVNGENISKLANSKQDVIYNELWPFGTSALGNRPQESYGDLKARVDQVRQATGKSLIVGAYMEEPKFDDNRIPLNGAARDVLASATYQTDAVLLTTAAIAAAGGYHMSLAALANPNDGGGVGVLETAYYPTQSLKVSKELNRKNYHYQQFITAYENLLRDKVENDSAEPQTFTANGRQLSQDALGINGDQVWTYAKKGNDFRTIQLLNLMGITSDWKNEDGYENNKTPDEQTNLLVTYPLTGVSMAEADRIAKQVYLTSPDDWLQSSMISLATQVKTNENGDPVLYIQVPRLTLWDMIYINETIKPETPKVPEQPQHPARTLEPAIPQTPEAVNPLPVANKQAVDENKNEIVSALTGEENDLQLPTLSKQSLPISQAELPQTGDNNETRSNLLKVIGAGALLIGAAGLLSLIKGRKND

SEQ ID NO: 36 - DNA, 3786 bp, Artificial Sequence
  Codon optimized Paenibacillus sp. mut gene DNA Sequence
atggcaggtggcccgaatcttactccaggtaaaccaattactgctagtggtcaatctcaaacctatagccctcaaaatgtaaaagatggcaatcaaaatacttactgggaaagtactaacaatgccttccctcaatggattcaagttgatttgggtgcaagtactggcattgatcaaattgttcttaagttaccagctagctgggaagctcgtactcaaactcttgctgttcaaggtagtttgaatggttctactttcactgatattgtaggttctgcaaattatgtattcagtccttctgtaggtaataacactgttactattaattttaccgccacaagcacccgttatgttcgcttgtacgtaactgcgaacactggttggccagctgctcaactgtctgaattagaaatttatggttctggtgaccagactcctgcacctgatacttatcaagctgaaagtgctgctttatctggtggcgctaaagtaaatactgatcatgccggctacataggtactggttttgttgatggttattggactcaaggcgctactactaccttttctgtaaacgcgcctactgctggtaattacgatgtaactctgaggtatggtaacgcaaccggcagtaataaaactgtatccttgtacgtaaatggcgctaaaattcgtcaaacaactttaccaagtctacctaactgggattcatggagtagcaagactgaaactcttaatttaaatgctggtagcaacaccattgcttataaatacgaccctggcgattctggtaatgtaaatcttgatcaaatcactgtagaagcatctacttcaactcctactcctactccatctcctactcctacacctactccaactcctactcctactcctactcctacaccaacacctactcctaccccaacccctactcctacacctacacctacacctactcctactcctcctcctggtggtaatattgccataggcaaatctatttccgcatctagtcacactcaaacttatgttgctgagaacgcaaatgataacgatgtaaatacttactgggaaggtggcggtaatcctagtactttaactttggatcttggcgctaattataatattacttctattgttctaaaactaaacccatcctctatatgggcagcccgtactcaaactattcaagttttgggccatgatcaaaatactactacattcagtaatttagtatctgctaaatcttactctttcgatcctgcttctggtaatactgttaccattccagttaccgctactgttaaacgtttgcagttgaacattacttctaattccggtgcccctgctggtcaagtagctgagttccaagttttcggtactcctgctccaaatcctgatttgactattaccggtatgtcttggtctccttcttctccagttgagacagatgcaattactctgaatgctactgttaaaaacaatggtaatgccagtgcagccgctaccaccgtaaatttctacctaaataacgagctagctggttctgctcctgtagcagctctagcggcaggcgcttctgcaactgttccgctaaatgtaggtgctaaaaccgccgccacatacgctgtaggtgctaaagtagatgaaagtaatgcagtaattgagttaaacgagtctaacaatagctacactaatcctgcttcattggttgttgctccagttagtagttctgatttagttggcactgtttcttggactccaagcactcctattgcaaacaatgctgtttcttttaacgtaaatcttaaaaatcaaggcactattgcttctgccggtggttctcacggtgttactgtagttcttaaaaatgcttccggttctaccgttcaaactttcagtggttcttacaccggtagtcttgctccgggagcttccgtaaatattacccttcctggtacctggactgctgctgctggtagctatactgtaactgcaaccgttgcggcagacgctaacgaacttcctatcaagcaagccaacaatgcaaacacagcaagtctaaccgtatattctgctcgtggtgcaagcatgccatacagtcgttacgataccgaggatgccacccttggtggtggcgctactctaaaatccgctccgacattcgatcaagcgcttactgcatctgaagccaccggtcaattgtacgctgcgttaccatctaacggctcttatcttcaatggaccgtacgtcaaggtcagggtggtgcaggcgttactatgagatttactatgccagattctgctgacggcatgggcttaaacggtagtttagatgtttacgtaaacggtacaaaagtaaaaaccgtatctctaaccagttactatagctggcagtatttctctggtgatatgccaggagacgctccaagcgctggtcgtcctttattccgttttgatgaagttcattggaaattagatactcctttgaaaccaggagatactattcgcatacaaaagaacaacggtgatagcctagaatacggtgtagactttattgaaattgaaccagttcctgctgctatctctcgtccggctaactctgtttccgtaactgattacggtgctgttcctaacgatggacaggacgatcttaccgcttttaaagcagccgtaaacgcagctgtagcatccgataaaatcttgtatattccagaaggcactttccacttgggtaacatgtgggagattggttccgtaagtaacatgatcgatcacattactattactggagctggtatttggtacactaacatccagtttaccaacgccaatcctgcttccggtggcatctctctacgtattactggtaaacttgatttcagcaacgtttacttgaactctaatttgcgttctcgttatggtcaaaatgccgtttataaaggttttatggataacttcggtaccaattccgtaattcgtgacgtatgggtagaacacttcgaatgtggtttctgggtaggtgattacggtcatactcctgctattcgcgcaagcggtctgttaattgaaaacagccgaatccgtaacaacctagctgatggtgtaaacttcgcccaaggtaccagcaattctaccgtacgcaacagcagcttacgtaacaacggtgatgacgcccttgctgtatggactagtaatactaacggtgctccagaaggcgtaaacaataccttctcttacaacaccatcgaaaacaactggcgcgctggaggtattgccttcttcggaggaagcggacataaggccgatcacaactacatagtagattgtgtaggtggttctggtatccgtatgaataccgttttcccaggatatcacttccagaacaataccggtattgttttctctgacactaccatagtaaactgcggtactagcaaagatctatacaacggtgaacgcggtgctatcgatttggaagcatctaacgacgccatcagaaacgttacttttaccaacatcgatattatcaactctcagcgcgatgctatccagttcggttatggtggtggtttcaccaatatcgttttcaacaacatcaacattaacggaaccggtcttgatggtgtaaccacctctcgtttctctggacctcatttaggcgcggcgatcttcacctataccggtaacggtagtgctactttcaacaatttacgcaccagcaatatcgcttatccaaatttatattatatccagagcggtttcaatttaatcatcaataattga

SEQ ID NO: 37 - protein, 1261 aa, Artificial Sequence
  Codon optimized Paenibacillus sp. mut gene amino acid Sequence
MAGGPNLTPGKPITASGQSQTYSPQNVKDGNQNTYWESTNNAFPQWIQVDLGASTGIDQIVLKLPASWEARTQTLAVQGSLNGSTFTDIVGSANYVFSPSVGNNTVTINFTATSTRYVRLYVTANTGWPAAQLSELEIYGSGDQTPAPDTYQAESAALSGGAKVNTDHAGYIGTGFVDGYWTQGATTTFSVNAPTAGNYDVTLRYGNATGSNKTVSLYVNGAKIRQTTLPSLPNWDSWSSKTETLNLNAGSNTIAYKYDPGDSGNVNLDQITVEASTSTPTPTPSPTPTPTPTPTPTPTPTPTPTPTPTPTPTPTPTPTPTPPPGGNIAIGKSISASSHTQTYVAENANDNDVNTYWEGGGNPSTLTLDLGANYNITSIVLKLNPSSIWAARTQTIQVLGHDQNTTTFSNLVSAKSYSFDPASGNTVTIPVTATVKRLQLNITSNSGAPAGQVAEFQVFGTPAPNPDLTITGMSWSPSSPVETDAITLNATVKNNGNASAAATTVNFYLNNELAGSAPVAALAAGASATVPLNVGAKTAATYAVGAKVDESNAVIELNESNNSYTNPASLVVAPVSSSDLVGTVSWTPSTPIANNAVSFNVNLKNQGTIASAGGSHGVTVVLKNASGSTVQTFSGSYTGSLAPGASVNITLPGTWTAAAGSYTVTATVAADANELPIKQANNANTASLTVYSARGASMPYSRYDTEDATLGGGATLKSAPTFDQALTASEATGQLYAALPSNGSYLQWTVRQGQGGAGVTMRFTMPDSADGMGLNGSLDVYVNGTKVKTVSLTSYYSWQYFSGDMPGDAPSAGRPLFRFDEVHWKLDTPLKPGDTIRIQKNNGDSLEYGVDFIEIEPVPAAISRPANSVSVTDYGAVPNDGQDDLTAFKAAVNAAVASDKILYIPEGTFHLGNMWEIGSVSNMIDHITITGAGIWYTNIQFTNANPASGGISLRITGKLDFSNVYLNSNLRSRYGQNAVYKGFMDNFGTNSVIRDVWVEHFECGFWVGDYGHTPAIRASGLLIENSRIRNNLADGVNFAQGTSNSTVRNSSLRNNGDDALAVWTSNTNGAPEGVNNTFSYNTIENNWRAGGIAFFGGSGHKADHNYIVDCVGGSGIRMNTVFPGYHFQNNTGIVFSDTTIVNCGTSKDLYNGERGAIDLEASNDAIRNVTFTNIDIINSQRDAIQFGYGGGFTNIVFNNININGTGLDGVTTSRFSGPHLGAAIFTYTGNGSATFNNLRTSNIAYPNLYYIQSGFNLIINN

SEQ ID NO: 38 - protein, 437 aa, Artificial Sequence
  amino acid sequence of the triacylglycerol lipase
MVSYVVALPEVMSAAATDVASIGSVVATASQGVAGATTTVLAAAEDEVSAAIAALFSGHGQDYQALSAQLAVFHERFVQALTGAAKGYAAAELANASLLQSEFASGIGNGFATIHQEIQRAPTALAAGFTQVPPFAAAQAGIFTGTPSGAAGFDIASLWPVKPLLSLSALETHFAIPNNPLLALIASDIPPLSWFLGNSPPPLLNSLLGQTVQYTTYDGMSVVQITPAHPTGEYVVAIHGGAFILPPSIFHWLNYSVTAYQTGATVQVPIYPLVQEGGTAGTVVPAMAGLISTQIAQHGVSNVSVVGDSAGGNLALAAAQYMVSQGNPVPSSMVLLSPWLDVGTWQISQAWAGNLAVNDPLVSPLYGSLNGLPPTYVYSGSLDPLAQQAVVLEHTAVVQGAPFSFVLAPWQIHDWILLTPWGLLSWPQINQQLGIAA